(19) United States
(12) Patent Application Publication
(10) Pub. No.: US 2023/0176060 A1
(43) Pub. Date: Jun. 8, 2023

(54) TUMOUR BIOMARKERS FOR IMMUNOTHERAPY

(71) Applicant: KYMAB LIMITED, Cambridge (GB)

(72) Inventors: Richard Charles Alfred SAINSON, Cambridge (GB); Cecilia DEANTONIO, Cambridge (GB); Chih-Hung HSU, Taipei City (TW); Li-Chun LU, Taipei City (TW); Lorcan Adrian SHERRY, Motherwell North Lanarkshire (GB)

(21) Appl. No.: 17/921,822

(22) PCT Filed: May 13, 2021
(86) PCT No.: PCT/EP2021/062778

§ 371 Date: Oct. 27, 2022

(30) Foreign Application Priority Data
May 14, 2020 (GB) 2007099.1

(51) Int. Cl. G01N 33/574; G01N 33/50

(52) U.S. Cl. CPC G01N 33/57484; G01N 33/5011

(57) ABSTRACT

Biomarkers for prognosis of tumours, in hepatocellular carcinoma and other cancers. Measurement of biomarkers for prescription of anticancer immunotherapy targeted against ICOS+ regulatory T cells (TReg), e.g., selecting patients for treatment with an anti-ICOS antibody. Biomarkers comprising: (i) ratio of the number of ICOS FOXP3 double positive cells within a defined radius of influence around ICOS single positive cells to the total number of ICOS single positive cells, (ii) mean distance between each ICOS positive FOXP3 negative cell and its nearest ICOS FOXP3 double positive cell, (iii) proportion of FOXP3 positive cells which are ICOS positive, and (iv) density of ICOS positive cells.

### FIELD OF THE INVENTION

The present invention relates to anti-cancer immunotherapy targeted against regulatory T cells expressing the T cell surface receptor ICOS (Inducible T cell Co-Stimulator), including anti-ICOS antibodies and other therapeutic agents that inhibit or kill ICOS+ regulatory T cells (TReg). The invention relates to biomarkers that indicate the susceptibility of a tumour to such immunotherapy and which provide an early indication of a patient's response to immunotherapy.

### BACKGROUND

Immunotherapy, where a patient's own immune system is modulated to combat disease, has risen to become a frontline treatment for many types of cancer. Signals that inhibit T cell activation are downmodulated using targeted drugs (“immune checkpoint blockade”), enabling the T cells to mount an effective anti-tumour response [1]. In patients who respond to immunotherapy, the anti-tumour results can be dramatic and life-saving, fueling interest in this field. Immunotherapy with immune checkpoint inhibitors such as monoclonal antibodies that target programmed cell death protein 1 (PD-1), programmed death ligand-1 (PD-L1), and cytotoxic T-lymphocyte-associated antigen 4 (CTLA-4) has become an important treatment for multiple cancers. Many hundreds of clinical trials are currently underway with immune checkpoint inhibitors to test new drugs and new drug combinations.

In addition to immune checkpoint co-inhibitory receptors such as PD-1 and CTLA-4, co-stimulatory receptors influence the functional status of T cells and are critical for cancer immune surveillance. Upon binding of ligands to the co-stimulatory receptors, downstream signals are activated, driving T cell function, survival and/or proliferation. ICOS is a co-stimulatory receptor found exclusively on T cells and has been identified as a target for immunotherapy to activate a patient's anti-tumour T cell response. See, e.g., WO2019/122884 and references therein.

In tumours, as well as in other diseases and conditions involving an immune component, a balance exists between effector T cells (TEff, such as CD8+ cytotoxic T lymphocytes, CTL) which exert an antigen-specific T cell immune response, and regulatory T cells (TReg) which suppress that immune response by downregulating TEffs. Elevated levels of TReg in tumours are linked to poor prognosis for at least some types of cancer, including hepatocellular carcinoma (HCC) [2, 3]. Tu et al (2016) reported that TReg, especially ICOS+FOXP3+ TReg, contribute to immunosuppression in HCC tumours and are linked to reduced patient survival [3]. ICOS is an important co-stimulatory receptor on TEff but also promotes tumour growth due to its high expression on TReg. In vitro studies have shown that ICOS-expressing (ICOS+) TReg are more immunosuppressive than ICOS-negative TReg [4, 5]. Anti-ICOS antibody immunotherapy aims to modulate the TEff/TReg balance in favour of TEff number and activity. Antibodies that trigger the depletion (e.g., via Fc-mediated effector function such as antibody dependent cell cytotoxicity, ADCC) of ICOS positive TReg would relieve the suppression of TEff and improve the TEff/TReg ratio, and would thus have a net effect of promoting the TEff anti-tumour response. Anti-ICOS antibody may also exert agonistic activity at the ICOS receptor level to directly stimulate cytokine production by TEff. A combination of these two TEff boosting effects is possible with an anti-ICOS antibody, due to ICOS being presented at significantly higher levels on TReg than TEff; e.g., human IgG1 anti-ICOS antibodies can boost anti-tumour immunity by stimulating ICOS^{Low} Teffs and depleting ICOS^{High} TRegs [6; 7]. Anti-ICOS antibodies thus represent a potentially valuable class of therapeutic agents for tumour immunotherapy in which a TEff immune response is mobilised. A number of anti-ICOS antibodies have been described and shown to influence T cell populations/activity [8; WO2016/120789; WO2016/154177; WO2018/029474; WO2018/187613; WO2019/122884]. Several anti-ICOS antibodies are undergoing clinical trials for in cancer patients: JTX-2011 [25], GSK-3359609 [9], MEDI-570, BMS-986226 [WO2018/187613; NCT03251924] and KY1044 [7].

Although the progress of immunooncology to date has been impressive, immunotherapeutic treatments still fail more patients than they save. Response rates of 30% or lower are the norm for immunotherapy across a range of tumour types. The response rate for the anti-PD-1 antibody nivolumab in melanoma is around 30%. In a phase I/II study in advanced HCC, its response rate was 15-20% [10]. Pembrolizumab, another anti-PD-1 antibody, achieved a similar response rate to nivolumab in a phase II study in patients with HCC who had previously been treated with sorafenib [11]. Nivolumab and pembrolizumab were granted accelerated approval for the treatment of HCC in 2017 and 2018, respectively, by the US Food and Drug Administration (FDA). However, subsequent phase Ill trials in advanced HCC testing nivolumab and pembrolizumab in 1st line and 2nd line setting, respectively, failed to achieve positive results [12, 13]. In a Phase II clinical trial of the anti-PD-L1 antibody atezolizumab in urothelial carcinoma, the response rate was around 26% in patients with PD-L1+ tumours, 15% overall (in patients regardless of PD-L1 expression on tumours), and 10% in patients with PD-L1 negative tumours. The latter example illustrates the value of a biomarker for matching an immunotherapy to a group of cancer patients who have a better chance (albeit still only 26%) of benefiting from it. Calderaro et al also reported relationships between PD-L1 expression in HCC and clinical and pathological features [14].

Combination therapies have been investigated as a way to improve the efficacy of immune checkpoint inhibitors in HCC and other cancers. A trial cohort combining nivolumab with the anti-CTLA-4 antibody ipilimumab recently reported an objective response rate around 30% [15], following which the US FDA granted an accelerated approval to the nivolumab/ipilimumab combination for HCC patients who have been previously treated with sorafenib. Another recent trial tested a combination of the anti-PD-L1 antibody atezolizumab and the anti-VEGF antibody bevacizumab, versus sorafenib as the first-line therapy for patients with advanced HCC, and the trial reported positive results with the combination of atezolizumab plus bevacizumab increasing the ORR to around 30% [16].

In general, it is unclear why some patients respond to immunotherapy while others do not. Dramatic differences in response are seen in ostensibly similar patients, where one patient responds completely while others experience little or no clinical benefit from the same treatment. This may be attributable to differences in the baseline tumour microenvironment (TME) but the details are not yet understood. While some predictive biomarkers have been identified (e.g., PD-L1 expression on tumour and immune cells in the atezolizumab study mentioned above), it remains the case that a large proportion of patients treated with immunotherapy receive no benefit from the treatment. Any advance in matching patients to the therapies that are most likely to work would bring a welcome improvement in clinical efficacy and limit the number of patients enduring fruitless medical interventions.

Traditional diagnosis of cancer types and sub-types is based on anatomical and tissue-based classifications, e.g., lung cancer, pancreatic cancer and so on. However, given the low response rates to immunotherapy within and across such tumour categories, these definitions are only of partial help in identifying groups of patients who will benefit from a particular treatment. Clinicians and regulators therefore now recognise the value of a “tissue-agnostic” approach whereby treatment is based on biomarkers of the tumour and/or patient which are separate from (but may correlate with) the tissue or cell type from which the tumour originates. For example, the US FDA has approved the anti-PD-1 antibody pembrolizumab for treatment of patients with unresectable or metastatic, microsatellite instability high or mismatch repair deficient solid tumours. A combination of tissue-type and tissue-agnostic tumour classifications is also possible.

The mutational status of tumours influences their microenvironment. Populations of tumour cells may evolve to produce phenotypes that shield them from immune detection and influence local tissues to support the tumour's growth, thereby establishing a pro-tumour environment. Such tumours may be poorly infiltrated by immune cells, and are referred to as being immunologically “cold”. In other situations, a high mutation rate in tumour cells (e.g., resulting from defects in DNA mismatch repair) generates an abundance of neoepitopes, leading to a high infiltration of cytotoxic T lymphocytes (CTL) and an immunologically “hot” tumour. The nature and extent of the connection between tumour genotype and phenotype, and their co-progression over time, are the subject of widespread and rapid discovery. It is hoped that the resolution of differences between responsive and non-responsive tumours will ultimately lead to the identification of many patient sub-classes for whom a particular course of immunotherapy is reliably curative. Since the majority of patients with metastatic tumours reportedly have numerous different genomic alterations, successful treatments may require customised combinations of multiple therapeutic agents to achieve a close match between the treatment and the cancer profile [17].

Various diagnostic systems have been developed based on characterisation of the TME including the immune infiltrate and local inflammatory response [18; 19; 20; 21]. Retrospective analyses of patient populations treated with immune checkpoint blockers such as anti-CTLA-4 and anti-PD-1 have indicated differences in the immune context of tumours linked with their ability to respond to treatment [21]. “Tumour mutational burden” and “T cell-inflamed gene expression profile” were reported to be associated with a positive treatment outcome with the anti-PD-1 antibody pembrolizumab [22]. The computational model “TIDE” (Tumour Immune Dysfunction and Exclusion), which models CTL evasion by tumours based on gene signatures (from pre-treatment RNA-Seq or NanoString profiles) correlating with CTL infiltration and patient survival, was reported to predict the outcome of melanoma patients treated with first line anti-PD-1 or anti-CTLA-4 more accurately than other biomarkers such as PD-L1 expression and mutation load [23]. The mode of action of the anti-CTLA-4 antibody ipilimumab was reported to involve FcγRIIIA-expressing non-classical monocytes, which were found at higher baseline peripheral frequencies in responding patients compared with non-responding patients [24].

Research indicates that anti-ICOS antibodies may offer particular therapeutic value for tumours that are positive for expression of ICOS and/or FOXP3 (a marker of TRegs) [6; WO2018/029474; WO2019/122884]. Based on data from the ICONIC trial NCT02904226 with the anti-ICOS antibody JTX2011, it was reported that rates of disease control and tumour reductions were higher in patients with high ICOS expression in the tumour [25]. Nevertheless, ICOS and/or FOXP3 expression provides only a “broad brush” indicator of whether an anti-ICOS antibody therapy will work for a given patient.

WO2014/009535 described a method for determining whether a patient suffering from solid cancer would respond to a treatment (chemotherapy, radiotherapy or immunotherapy), involving assaying a tumour sample from the patient for the expression levels of a set of genes selected from CCR2, CD3D, CD3E, CD3G, CD8A, CXCL10, CXCL11, GZMA, GZMB, GZMK, GZMM, IL15, IRF1, PRF1, STAT1, CD69, ICOS, CXCR3, STAT4, CCL2, and TBX21. Expression levels of genes in this set were also reported to be linked with cancer prognosis.

WO2014/023706 described a method for determining whether a cancer patient had a good or bad adaptive immune response and a good or bad immunosuppressive response, involving assaying a tumour sample from the patient for the expression levels of a defined gene set.

WO15/103037 described methods of assaying cancer samples for somatic mutations to identify patients who were candidates for treatment with an immune checkpoint modulator.

WO16/109546 described selection of patients for treatment with immunotherapy based on an “immune cell gene signature” in a biological sample obtained from the patient, the immune cell signature comprising the expression level of one or more of genes from a defined gene set.

WO2017/070423 described a method of assaying patient samples for levels of mRNA from a defined gene set to identify patients for treatment with an anti-ICOS antibody.

WO2018/225062 and WO2018/225063 described a method of predicting the response of a cancer patient to treatment with at least an immune checkpoint inhibitor, comprising assaying “host driven” biomarkers such as cytokines, chemokines, growth factors, enzymes or soluble receptors.

WO2020/245155 described a method for determining a treatment regimen with a chemotherapeutic agent in a patient affected with a cancer, the method comprising quantifying CD8 and CD3 in a tumour sample from the patient. Methods of quantifying density of CD8+ and CD3+ cells in the tumour and in the invasive margin of the tumour were described.

As well as identifying suitable biomarkers that enable the prescription of appropriate treatments and treatment combinations for individual patients, it is useful to identify prognostic biomarkers for patients who are at an early stage of treatment. Immunotherapy treatment regimens have a long duration, lasting at least months and often years. This is unlike chemotherapy treatment where cell killing/tumour shrinkage is almost immediate. The response to immunotherapy can be considered a three-step process: a cellular response as the drug activates cells of the immune system; an anti-tumour response as the immune cells attack the tumour; and a therapeutic response as the anti-tumour effect reduces the tumour burden and improves the outcome for the patient. If one can identify early markers which correlate with the eventual therapeutic response, such as biomarkers that can be detected in patients within the first few weeks of treatment and which indicate the longer-term prognosis, this would provide confidence in continued treatment for patients exhibiting a positive biomarker whereas patients lacking the biomarker may be switched to alternative therapies.

Feng et al [26] reported that prognostic information for patients with oral squamous cell cancer could be derived from the frequency of T cells in tumour tissue sections. High numbers of CD8+ T cells at the invasive margin was positively correlated with overall survival, while the number of FOXP3+ cells within 30 um of CD8+ T cells was negatively correlated with overall survival. The authors integrated these and other measurements into a “cumulative suppression index” correlating with overall survival.

WO2019/222188 identified elevated levels of ICOS and T-bet as being linked with patient response to anti-ICOS antibody.

Recently, Kagamu et al [27] reported that the status of CD4+ T cells in the peripheral blood of patients could be used to identify patients with non-small cell lung cancer who presented early disease progression after treatment with nivolumab (anti-PD-1, enabling classification of patients as non-responders or responders. Responders were found to have significantly (p<0.0001) higher percentages of effector, CD62L^{low} CD4+ T cells prior to PD-1 blockade. Conversely, the percentage of CD25+ FOXP3+ CD4+ T cells was significantly (p=0.034) higher in non-responders. Gene expression analysis revealed that CCL19, CLEC-2A, IFNA, IL7, TGFBR3, CXCR3, and HDAC9 were preferentially expressed in CD62L^{low} CD4+ T cells derived from responders. Notably, long term responders, who had >500-day progression-free survival, showed significantly higher numbers of CD62L^{low} CD4+ T cells prior to PD-1 blockade therapy. Decreased CD62L^{low} CD4+ T-cell percentages after therapy resulted in acquired resistance, with long term survivors maintaining high CD62L^{low} CD4+ T-cell percentages.

### SUMMARY OF THE INVENTION

We have discovered that features of the cellular composition, immune contexture and specific spatial arrangement of cells within the tumour microenvironment (TME) can inform a patient's likelihood of clinical progression and whether they are likely to benefit from treatment with immunotherapy. We have identified biomarkers of the TME that correlate with duration of patient survival and likelihood of response to immunotherapy, including treatment with an anti-ICOS and/or anti-TReg immunotherapeutic agent. These biomarkers assist with disease prognosis in cancer patients, allow patients to be profiled to identify their likelihood of benefiting from immunotherapy and, when monitored both before and after treatment, provide a valuable early indication of whether a patient is responding to the therapy.

These biomarkers include the following features of the TME, which may be identified from a patient's tumour, e.g., via analysis of a resected tumour or biopsy sample:

(i) ICOS+ density: the density or concentration of cells that express ICOS protein, i.e., ICOS positive (ICOS+) cells,
(ii) ICOS+ TReg proportion: the proportion of FOXP3+ cells which are ICOS+, representing the proportion of TReg that are ICOS positive,
(iii) Intercellular proximity: ICOS+ FOXP3+: ICOS+ FOXP3− intercellular proximity, being the average (mean) distance between each ICOS single positive (i.e., ICOS positive FOXP3 negative) cell and its nearest ICOS FOXP3 double positive cell, representing the proximity between ICOS positive TReg and other ICOS positive cells (including ICOS+ TEff), and
(iv) Zonal influence ratio: the ratio of the number of ICOS FOXP3 double positive cells within a defined region (“radius of influence”) of any ICOS single positive cell to the total number of ICOS single positive cells, wherein the radius of influence represents a distance (e.g., 30 μm) across which cell-cell and/or cytokine-dependent communication can occur between an ICOS FOXP3 double positive cell and an ICOS single positive cell.

Each of these biomarkers correlates positively with the expectation that the patient will benefit from the anti-ICOS and/or anti-TReg immunotherapy. In general, the higher the biomarker (the greater the density, proportion, proximity or zonal influence), the poorer the prognosis for the patient in the absence of treatment, but the greater likelihood that the patient will respond to the anti-ICOS and/or anti-TReg intervention. Thus, measurement of biomarkers as described herein facilitates the appropriate selection of patients for treatment, enabling immunotherapies to be selectively administered to those patients in whom it is most likely to generate a beneficial anti-tumour response.

Details of these biomarkers are summarised in appended Table B and described herebelow. Biomarkers (ii), (iii) and (iv) are indicators of an immunosuppressive TME.

FOXP3 is a known marker of TReg, and cells which express both ICOS and FOXP3 are identifiable as a highly immunosuppressive sub-group of TReg. While we describe herein the use of FOXP3 as an identifying marker for TReg, it will be appreciated that alternative markers could readily be used for selectively identifying TReg. As we disclose herein, a greater density of ICOS+ cells, a greater proportion of TReg being ICOS+, and a closer proximity between ICOS+ TReg and other ICOS+ T cells (FOXP3−), are all associated with a worse prognosis for the patient, manifesting e.g., as reduced duration of survival or reduced duration of recurrence free survival (RFS), progression free survival (PFS) or time to progression (TTP), relative to other patients with the same tumour type. On the upside, however, such patients represent a subgroup who are especially likely to respond to anti-ICOS and/or anti-TReg immunotherapy.

Treatment with an anti-TReg therapeutic agent, such as an Fc effector positive anti-ICOS antibody like the KY1044 antibody described herein, reduces the number of TReg, improves the ratio between TEff/TReg in the TME and may thereby boost the patient's immune response against the tumour, leading to reduced tumour growth and preferably to reduction in size and eventual eradication of the tumour or tumours in the patient. Other, non TReg-depleting, anti-ICOS antibodies would similarly improve the anti-tumour immune response by enhancing cytokine production by TEff and thereby boosting TEff activity.

We describe herein how the defined biomarkers may be measured and quantified in individual tumour samples and in patient groups, providing cut-off values which usefully classify patients into responder vs non-responder groups. Given the complex nature of tumour biology and the diversity of patients, the predictions made using these biomarkers cannot be 100% accurate, but they will still provide a useful guide based on probabilities. Thus, the invention increases the probability of suitably matching a patient to a therapy from which that patient will derive benefit. Cancer patients may thus be screened using biomarkers according to the present invention to provide an indication of the relative likelihood of response to therapy, this information being valuable to both clinicians and patients in deciding whether to embark on a course of immunotherapy according to the present invention and/or in determining what course of immunotherapy (e.g., which monotherapy or which combination of agents) to adopt.

Embodiments of the invention are disclosed with reference to hepatocellular cancer (HCC), exemplified by the following quantitative measures in the TME base on a studied patient cohort:

High density of ICOS+ cells, measured as more than 120 cells per mm²;

High density of ICOS+ cells, more than 100 cells per mm² where the HCC is known to be associated with hepatitis B virus (HBV) infection OR is stage 2 or later HCC according to the criteria of the American Joint Committee on Cancer (AJCC) [28];

High proportion of FOXP3+ cells which are ICOS+, measured as more than half of FOXP3+ cells being ICOS+;

Close proximity between ICOS+ FOXP3− negative (ICOS single positive) cells and their nearest neighbour ICOS+ FOXP3+ double positive cells, measured as a mean intercellular distance of less than 105 μm;

High ratio of the number of ICOS FOXP3 double positive cells within a 30 μm radius of influence of ICOS single positive cells to all ICOS single positive cells.

HCC patients meeting one or more (preferably all) of these criteria may be selected for treatment with anti-ICOS and/or anti-TReg immunotherapy as described herein.

Reference values will normally be determined with respect to patients with particular clinical characteristics (such as cancer sub-type) and are best used for prognosis in comparable patients. The above are illustrative embodiments for HCC patient groups exemplified herein, and other suitable cut-off values may be determined with reference to other tumour types and/or populations of patients. Thus, although reference values exemplified herein may optionally be applied for prognosis in patients with other cancers besides HCC, their use in such wider contexts should first be confirmed, if possible, by evaluation of data from patients with the target cancer type or molecular subtype (histology agnostic). We describe methods of determining suitable reference values and formulae for differentiating patients according to predicted cancer prognosis and likely benefit from anti-ICOS and/or anti-TReg immunotherapy. The determination and use of reference values for HCC is illustrated in the Examples and equivalent methods may be used to determine and deploy reference values with respect to other cancers.

The present invention relates to the application of the biomarkers in medical contexts, including in the following aspects:

in the prognosis of cancer in patients, e.g., to predict survival, which may be length of survival, recurrence free survival (RFS), progression-free survival (PFS) and/or time to progression (TTP);

in the matching of patients to appropriate treatments, e.g., selecting patients for treatment with anti-ICOS and/or anti-TReg immunotherapy and prescribing anti-ICOS and/or anti-TReg immunotherapy for patients who are identified as being more likely to benefit;

in monitoring the cellular response to anti-ICOS and/or anti-TReg immunotherapy as an early indicator of whether a patient is responding to the treatment;

in medical research, wherein biomarker data from patients are mapped against their disease history and/or response to therapy, to provide or refine models that can improve clinical assessment and treatment for future patients.

One or more biomarkers may be used for prognosis of a cancerous solid tumour in a patient, including one or more of the following biomarkers as determined in tumour core tissue from the patient:

(i) ratio of the number of ICOS FOXP3 double positive cells within a defined radius of influence around ICOS single positive cells to the total number of ICOS single positive cells,

(ii) mean distance between each ICOS positive FOXP3 negative cell and its nearest ICOS+ FOXP3+ double positive cell,

(iii) proportion of FOXP3 positive cells which are ICOS positive, and

(iv) density of ICOS positive cells.

Accordingly, in a first aspect, the invention provides a method for prognosis of a cancerous solid tumour in a patient, comprising

providing a sample of tumour core tissue obtained from the patient,

determining one or more of the following biomarkers in said sample:

(i) ratio of the number of ICOS FOXP3 double positive cells within a defined radius of influence around ICOS single positive cells to the total number of ICOS single positive cells,

(ii) mean distance between each ICOS positive FOXP3 negative cell and its nearest ICOS+ FOXP3+ double positive cell,

(iii) proportion of FOXP3 positive cells which are ICOS positive, and

(iv) density of ICOS positive cells, and

providing a prognosis for the patient based on said one or more biomarkers, wherein a shorter survival is indicated by

a greater ratio of the number of ICOS FOXP3 double positive cells within a defined radius of influence around ICOS single positive cells to the total number of ICOS single positive cells,

a shorter mean distance between each ICOS positive FOXP3 negative cell and its nearest ICOS+ FOXP3+ double positive cell,

a higher proportion of FOXP3 positive cells which are ICOS positive, and/or

a higher density of ICOS positive cells.

The ratio of the number of ICOS FOXP3 double positive cells within a defined radius of influence around ICOS single positive cells to the total number of ICOS single positive cells may be determined and compared against a reference value. A number higher than the reference value indicates a prognosis of shorter survival and a number lower than the reference value indicates a prognosis of longer survival. In HCC, for example, we determined a reference value for patient classification to be 0.1 for a 30 μm (30 micrometre) radius of influence. Thus, where there is determined to be a ratio of more than 0.1 ICOS FOXP3 double positive cells within a defined radius of influence around ICOS single positive cells to the total number of ICOS single positive cells, this is indicative of shorter survival.

Similarly, the mean distance between each ICOS+ FOXP3− (ICOS single positive) cell and its nearest ICOS+ FOXP3+(ICOS FOXP3 double positive) cell may be determined and compared against a reference value. A distance less than the reference value indicates a prognosis of shorter duration of survival, whereas a distance more than the reference value indicates a prognosis of longer survival. For example, where the tumour is HCC, a reference value of 105 μm may be used. Thus, a mean distance lower than 105 μm between each ICOS single positive cell and its nearest ICOS+ FOXP3+ double positive cell is indicative of shorter survival in HCC, whereas a mean greater than 105 μm between each ICOS single positive cell and its nearest ICOS+ FOXP3+ double positive cell is indicative of longer survival in HCC.

The proportion of FOXP3 positive cells which are ICOS positive may be determined and compared against a reference value. A proportion higher than the reference value indicates a prognosis of shorter survival, whereas a proportion lower than the reference value indicates a prognosis of longer survival. For example, where the tumour is HCC, a reference value of 0.5 may be used. Thus, if over half of FOXP3 positive cells are ICOS positive this is indicative of shorter survival, whereas if under half of FOXP3 positive cells are ICOS positive this is indicative of longer survival.

The density of ICOS positive cells may be determined and compared against a reference value. A density higher than the reference value indicates a prognosis of shorter survival, whereas a density lower than the reference value indicates a prognosis of longer survival. For example, where the tumour is HCC, a reference value of 120 ICOS positive cells per mm² may be used. Thus, a density of ICOS+ cells that is greater than 120 per mm² is indicative of shorter survival, whereas a density lower than 120 per mm² is indicative of longer survival. Where the tumour is HCC associated with hepatitis B virus infection or is stage 2 or later HCC, a reference value of 100 ICOS positive cells per mm² may be used. Prognosis for a patient presenting with these sub-types of HCC may therefore use this lower reference value, whereas prognosis for a patient presenting with another type of HCC or with HCC that has not been identified as being associated with HBV or as stage 2 or later HCC may use the higher reference value of 120 cells per mm². The latter includes patients diagnosed with stage 1 HCC.

As will be illustrated by the accompanying Examples, reference values are derived from statistical modelling and, while a reference value may represent a “best fit” to the data in the model from which it has been derived, it can only serve as an approximate guide to patient classification and prognosis and so should not be regarded as absolutely determinative. It will be understood that the exact values provided herein, such as “0.1 ICOS FOXP3 double positive cells within a 30 μm radius”, “mean distance of 105 μm between each ICOS+ FOXP3− cell and its nearest ICOS+ FOXP3+ cell”, “50% FOXP3 positive cells being ICOS positive”, “120 ICOS+ cells per mm²” or “100 ICOS+ cells per mm²” represent illustrative embodiments only and that the invention may be practised using variants of these precise values while still retaining predictive value. For example, if one were to define the radius of influence as 25 μm and the threshold number of ICOS FOXP3 double positive cells as 0.2, one would still expect to be able to usefully estimate whether a patient was one for whom a relatively long survival could be expected and whether such a patient would be more or less likely to benefit from treatment with an anti-ICOS and/or anti-TReg therapeutic agent.

Survival may be defined and measured in different ways. Most simply, survival may be defined as duration of overall survival (OS), i.e., the length of time until the patient dies. Other survival-related clinical endpoints incorporate a measure of the status of the patient's disease over the measured survival time, such as include recurrence free survival (RFS), progression free survival (PFS) and time to progression (TTP). In brief, RFS is the length of time until a treated cancer relapses or returns, PFS is the length of time until the cancer worsens (progresses) and TTP is similar to PFS but does not count patients who die from causes unrelated to their cancer. The biomarkers of the present invention are predictive of the patient's ability to survive the disease, and are thus expected to correlate with the duration of each of OS, RFS, PFS and TTP. In this context, the predicted duration of survival is survival in the absence of treatment with an anti-ICOS and/or anti-TReg immunotherapeutic agent, i.e., the prognosis is made without regard to how or whether the patient will receive anti-ICOS and/or anti-TReg immunotherapy as described elsewhere herein.

Providing the prognosis may thus comprise predicting whether the patient will enjoy a longer survival relative to other comparable patients. Providing the prognosis may comprise predicting survival time, such as predicting duration of OS, RFS, PFS and TTP, for example as an estimated number of months or years. A patient's predicted duration of survival (e.g., OS) is optionally estimated as a number of months from the date on which the tissue sample was taken. Duration of survival may be estimated as a range, for example less than x months or more than x months, according to whether the biomarker(s) fall above or below their corresponding reference value(s). For example, in an HCC patient population described herein, median OS was 100.3 months. For a further patient presenting with HCC, biomarker readings for zonal influence, intercellular proximity, ICOS+ TReg proportion and/or ICOS+ cell density would be determined and compared against the reference values defined for this population in order to provide a prognosis of survival being greater than or less than 100.3 months.

A patient who is thus identified as having a relatively poor prognosis as predicted by one or more such biomarkers may have a greater probability of responding to immunotherapy according to the present invention than patients for whom the prognosis is more optimistic. In other words, although the patient is predicted to have a relatively short duration of survival, it is more likely that the patient's survival can be extended by treatment with an anti-ICOS and/or anti-TReg immunotherapeutic agent.

Thus, a second aspect of the present invention relates to identifying patients who are more likely than others to respond to treatment with an anti-ICOS and/or anti-TReg immunotherapeutic agent. This aspect of the invention provides for the identification of a patient subgroup (e.g., a subgroup of HCC patients), wherein the subgroup is defined by the presence or value of a biomarker or combination of biomarkers, and wherein patients within the said subgroup are predicted to be more responsive to anti-ICOS and/or anti-TReg immunotherapy than patients outside the subgroup (i.e., compared with patients who do not exhibit the defined biomarker(s)).

In this second aspect, the present invention provides a method of determining the likelihood of a cancerous solid tumour in a patient to respond to an anti-ICOS and/or anti-TReg immunotherapeutic agent, comprising

providing a sample of tumour core tissue obtained from the patient, and

determining one or more of the following biomarkers in said sample:

(i) ratio of the number of ICOS FOXP3 double positive cells within a defined radius of influence around ICOS single positive cells to the total number of ICOS single positive cells,

(ii) mean distance between each ICOS+ FOXP3− cell and its nearest ICOS+ FOXP3+ cell,

(iii) proportion of FOXP3 positive cells which are ICOS positive, and

(iv) density of ICOS positive cells, wherein

a greater likelihood of the patient to respond to the anti-ICOS and/or anti-TReg immunotherapeutic agent is indicated by

a greater ratio of the number of ICOS FOXP3 double positive cells within a defined radius of influence around ICOS single positive cells to the total number of ICOS single positive cells,

a shorter mean distance between each ICOS positive FOXP3 negative cell and its nearest ICOS+ FOXP3+ positive cell,

a higher proportion of FOXP3 positive cells which are ICOS positive, and/or

a higher density of ICOS positive cells.

One or more of the above biomarkers is thus used for determining the likelihood of a cancerous tumour in a patient to respond to an anti-ICOS and/or anti-TReg immunotherapeutic agent. It may be determined whether the patient has an increased or reduced likelihood of responding to the treatment relative to other patients and/or the likelihood of response may be numerically estimated.

Optionally, the method comprises comparing the one or more biomarkers against corresponding reference values, in the same way as described for the first aspect of the invention and as exemplified elsewhere herein. Reference values are calculated for a reference population of patients, e.g., biomarkers determined for an HCC patient may be compared against reference values from a population of HCC patients. An increased or reduced likelihood of responding to the treatment is thus determined in relation to the expectation of response to treatment in the reference population.

Methods may further comprise identifying a patient as having an increased likelihood of responding to the immunotherapy and thereby selecting an anti-ICOS and/or anti-TReg immunotherapeutic agent for treatment of said patient. One or more of the above biomarkers may thus be used to identify a patient as being more likely to benefit from anti-ICOS and/or anti-TReg immunotherapy, and optionally selecting a patient for treatment with an anti-ICOS and/or anti-TReg immunotherapeutic agent accordingly. For example, a patient with HCC who shows an increased number of ICOS+ cells in the TME, exceeding a defined reference value, may be identified as being suitable for treatment with an anti-ICOS and/or anti-TReg immunotherapeutic agent. Optionally, the anti-ICOS and/or anti-TReg immunotherapeutic agent is then prescribed for and/or administered to the patient.

Where biomarker readings indicate that the patient is less likely to respond to the immunotherapy (a non-responder), this patient may be recommended a different treatment. Alternatively, such biomarker readings may indicate that the patient should be treated with the anti-ICOS and/or anti-TReg immunotherapeutic agent in combination with one or more other treatments, in which combination a response is more likely to be attained. For example, some treatments may influence the biomarkers described herein, and may result in the biomarkers changing in a direction that indicates an increased role for immunotherapy. The immunotherapy may be administered in combination with this further treatment, the multiple treatments being administered either together (simultaneously) or sequentially, in any order. FIG. 1.

In a third aspect, the present invention relates to treatment of cancer patients with anti-ICOS and/or anti-TReg immunotherapy wherein the patients are identified by the biomarkers described herein, and to anti-ICOS and/or anti-TReg immunotherapeutic agents for use in treating patients identified by the biomarkers described herein. Pharmaceutical compositions comprising the anti-ICOS and/or anti-TReg immunotherapeutic agents may be provided for use in such patients. Further, an anti-ICOS and/or anti-TReg immunotherapeutic agent may be used for the manufacture of a medicament for treatment of a cancerous solid tumour in such a patient. The treatment may comprise extending survival of the patient.

A method according to this third aspect of the invention comprises treating a cancerous solid tumour in a patient, wherein the method comprises

identifying a patient as having an increased likelihood of responding to an anti-ICOS and/or anti-TReg immunotherapeutic agent, wherein said patient is or has been identified as such as described in the second aspect of the invention. Thus for example the patient may be identified by one or more of the following biomarker readings from the tumour:

a ratio of the number of ICOS FOXP3 double positive cells within a defined radius of influence around ICOS single positive cells to the total number of ICOS single positive cells, wherein said ratio is higher than a reference value,

a mean distance between each ICOS positive FOXP3 negative cell and its nearest ICOS positive FOXP3 positive cell, wherein said distance is less than a reference value,

a proportion of FOXP3 positive cells which are ICOS positive, wherein said proportion is higher than a reference value, and

a density of ICOS positive cells, wherein said density is higher than a reference value, and

administering an anti-ICOS and/or anti-TReg immunotherapeutic agent to the patient.

The anti-ICOS and/or anti-TReg immunotherapeutic agent is thus used to treat a cancerous solid tumour in a patient, wherein the tumour has been determined to comprise one or more of the defined biomarkers. Accordingly, prior to treatment with the anti-ICOS and/or anti-TReg immunotherapeutic agent, one or more of the biomarkers in a sample of tumour core tissue previously obtained from the patient may have been determined to indicate suitability of the therapy by comparison to a reference value for the biomarker as described herein. For HCC as an example, the tumour may have been determined to comprise one or more of the following biomarkers:

a ratio of the number of ICOS FOXP3 double positive cells within a defined radius of influence around ICOS single positive cells to the total number of ICOS single positive cells, wherein said ratio is higher than 0.1,

a mean distance between each ICOS positive FOXP3 negative cell and its nearest ICOS positive FOXP3 positive cell, wherein said distance is less than 105 μm,

a proportion of FOXP3 positive cells which are ICOS positive, wherein said proportion is higher than half, and

a density of ICOS positive cells, wherein said density is higher than 120 cells per mm².

For HCC associated with HBV, or for stage 2 or later HCC, the tumour may have been determined to comprise one or more of the following biomarkers:

a ratio of the number of ICOS FOXP3 double positive cells within a defined radius of influence around ICOS single positive cells to the total number of ICOS single positive cells, wherein said ratio is higher than 0.1,

a mean distance between each ICOS positive FOXP3 negative cell and its nearest ICOS positive FOXP3 negative cell, wherein said distance is less than 105 μm,

a proportion of FOXP3 positive cells which are ICOS positive, wherein said proportion is higher than half, and

a density of ICOS positive cells, wherein said density is higher than 100 cells per mm².

A fourth aspect of the invention relates to measuring the response of a cancer patient to anti-ICOS and/or anti-TReg immunotherapy by detecting change in one or more biomarkers described herein. Such changes can represent a signature of response, and may be detectable significantly earlier than outwardly visible clinical signs, providing a useful indication of whether the treatment is achieving biological effects that will inhibit progression of the disease.

A method of monitoring a patient's response to an anti-ICOS and/or anti-TReg immunotherapeutic agent which has been administered to treat a cancerous solid tumour may comprise

providing a test sample of tumour core tissue obtained from a patient after administration of the anti-ICOS and/or anti-TReg immunotherapeutic agent,

determining one or more of the following biomarkers in said sample:

(i) ratio of the number of ICOS FOXP3 double positive cells within a defined radius of influence around ICOS single positive cells to the total number of ICOS single positive cells,

(ii) mean distance between each ICOS+ FOXP3− cell and its nearest ICOS+ FOXP3+ cell,

(iii) proportion of FOXP3 positive cells which are ICOS positive, and

(iv) density of ICOS positive cells,

comparing said one or more biomarkers in said test sample against the same one or more biomarkers in an earlier sample of tumour core tissue obtained from the patient, and

determining whether a change has occurred in said one or more biomarkers.

The test sample is obtained after administering the immunotherapeutic agent, after a allowing a period of time for the immunotherapeutic agent to take effect. For example, the test sample may be taken at least 3 days after said administration of the immunotherapeutic agent. The test sample is preferably taken within 2, 4, 6 or 8 weeks of said administration.

The earlier sample is obtained from the patient at an earlier time point in treatment of the patient, before the time of obtaining the test sample and preferably prior to the said administration of the immunotherapeutic agent. The earlier sample may be obtained prior to (optionally soon before, e.g., up to 14 days previously), at the time of (e.g., on the same day), or shortly after (e.g., the day after), the said administration of the immunotherapeutic agent. The earlier sample may be an initial sample obtained prior to or at the commencement of a course of treatment with the immunotherapeutic agent, (e.g., up to 14 days before), at the time of (e.g., on the same day), or shortly after (e.g., the day after), an initial administration of the immunotherapeutic agent.

The test sample is typically obtained at least 2 weeks after the earlier sample against which it is compared.

It can thereby be assessed whether the patient is responding to the immunotherapeutic agent, wherein a response is indicated by one or more of the following changes from the earlier sample:

a reduction in the ratio of the number of ICOS FOXP3 double positive cells within a defined radius of influence around ICOS single positive cells to the total number of ICOS single positive cells,

an increase in the mean distance between each ICOS+ FOXP3− cell and its nearest ICOS+ FOXP3+ cell,

a reduction in the proportion of FOXP3 positive cells which are ICOS positive, and

a reduction in the density of ICOS positive cells.

In effect, the earlier sample is used to provide reference values for the one or more biomarkers, against which the one or more biomarkers from the test sample are then compared.

The method may comprise observing change in one or more of said biomarkers in the test sample compared with the earlier sample, wherein said change is indicative that the patient is responding to the immunotherapy.

Methods of this fourth aspect of the invention may be used in continuous assessment or monitoring of patients undergoing anti-ICOS and/or anti-TReg immunotherapy, and thus are optionally repeated periodically during the course of therapy, in which the readings for the one or more biomarkers are compared against the previous readings, thereby charting change in the one or more biomarkers over time. In addition to an initial sample which is preferably obtained before the first administration of the anti-ICOS and/or anti-TReg immunotherapeutic agent to the patient, samples may optionally be taken for the measurement of biomarkers after every administration of the anti-ICOS and/or anti-TReg immunotherapeutic agent, or only after certain administrations (for instance, after the first administration and again at approximately monthly, two monthly or three monthly intervals, wherein tissue sampling is optionally timed to coincide with patient visits to the clinic for treatment). Readings from a test sample may thus be compared against those from multiple earlier samples in order to monitor changes in biomarkers over an extended period of time. Methods may therefore comprise detecting changes in one or more of the biomarkers in the test sample compared with the earlier sample, said changes optionally being detected in a series of multiple samples (comparing the test sample against multiple earlier samples, e.g., samples over an extended period of weeks or months), and detecting a response signature (preferably, a sustained response signature observed in the test sample compared against multiple earlier samples), wherein the response signature indicates that the patient is responding to the treatment.

Biomarker readings may be used to inform clinical decisions concerning the further treatment of the patient. Where positive signals are detected (the one or more biomarkers indicating that the patient is responding to the treatment), the anti-ICOS and/or anti-TReg immunotherapy may be continued. If this is not the case, or if it ceases to be the case during prolonged treatment, then the immunotherapy may be terminated and the patient may optionally be switched to an alternative treatment. Alternatively, supplementation of the immunotherapy with one or more additional treatments may be indicated. Optionally, selection of such alternative treatment is guided by biomarkers determined in the samples. Of course, any such treatment decisions will also consider any other symptoms or signs of disease or clinical response in the patient, but the biomarker readings present a valuable and early insight into the pharmacodynamics of the treatment and thus provide an important signal to review and update a patient's prognosis and to guide decision-making by the physician.

In general it is expected that changes in multiple biomarkers will trend in the same direction as one another, e.g., all changes indicating response to treatment, rather than one biomarker strongly indicating response to change and another biomarker indicating the opposite. Greater accuracy and greater predictive value may be obtained by integrating the output of multiple biomarker readings according to a formula, where the output of the formula is compared between samples and/or against reference values, as described elsewhere herein.

A response signature may be observed, wherein one or more biomarker readings or a formula calculated therefrom indicates response to treatment, the response signature being identified by

measuring a reduction in the ratio of the number of ICOS FOXP3 double positive cells within a defined radius of influence around ICOS single positive cells to the total number of ICOS single positive cells,

measuring an increase in the mean distance between each ICOS single positive cell and its nearest ICOS FOXP3 double positive cell,

measuring a reduction in the proportion of FOXP3 positive cells which are ICOS positive, and/or

measuring a reduction in the density of ICOS positive cells.

The change may be identified by comparing biomarker readings before and after therapy.

On observing the presence of a response signature in a test sample, continued treatment with the anti-ICOS and/or anti-TReg immunotherapeutic agent may be prescribed for the patient.

The degree of change in the biomarkers may be used to inform dosing and/or scheduling of therapy. Treatment may be iteratively adjusted to maximise positive change in the biomarkers, i.e., change indicative of response to treatment, with quantity and/or timing of the administered therapeutic agent being adjusted on the basis of the most recent biomarker data.

If there is no positive change in the biomarkers, i.e., the biomarkers do not indicate that the patient is responding to treatment, especially if no positive change is observed over prolonged monitoring with repeated sampling, then anti-ICOS and/or anti-TReg cancer immunotherapy treatment may be discontinued for said patient or the treatment regimen may be changed, e.g., from monotherapy treatment to a combination treatment.

The signature of response may indicate that the anti-ICOS and/or anti-TReg immunotherapeutic agent has predisposed the patient to respond to other treatment such as immune checkpoint blockers. This allows a tailored approach to combination therapies, whereby a further therapeutic agent is selectively prescribed for treatment of a patient in whom a response signature is detected. Changes in one or more of the biomarkers may indicate for example that a tumour has an increased susceptibility to other treatment (besides the existing anti-ICOS and/or anti-TReg immunotherapy) such as administration of a further therapeutic agent, and the biomarkers may be monitored to determine an appropriate timing of administration for such other treatment. Examples of such other treatment include administration of a different anti-ICOS and/or anti-TReg immunotherapeutic agent, an immune checkpoint blocker, a chemotherapeutic agent, targeted therapy (such as anti-angiogenic and tyrosine kinase inhibitors), or radiotherapy (or combinations of multiple such other treatments). Agents which induce immunological cell death may be used. Any such other treatment may be administered to a patient in combination with the previous anti-ICOS and/or anti-TReg immunotherapeutic agent (i.e., the previous treatment is not discontinued) or may substitute for it (i.e., the previous treatment is discontinued, the patient being switched over to the other treatment).

In a fifth aspect, the present invention relates to determining parameters for using the biomarkers in new patient subgroups, including correlating one or more biomarkers with patient prognosis or response to treatment, and identifying quantitative values for the biomarker which allow patients to be differentiated with respect to the tumour prognosis or response to treatment. The reference values thus determined may be employed in other aspects of the invention, such as disease prognosis and selection of patients for treatment.

This fifth aspect of the present invention provides a method of identifying a reference value for classifying patients with cancerous solid tumours according to their predicted prognosis or predicted response to an anti-ICOS and/or anti-TReg immunotherapeutic agent, comprising

providing samples of tumour core tissue obtained from each of a population of patients (a statistically meaningful number e.g., at least 20, at least 100) with a single tumour type (cancerous solid tumours of the same histological and/or molecular type (optionally histology agnostic) for whom disease outcome is known or response to the anti-ICOS and/or anti-TReg immunotherapeutic agent is known,

determining one or more of the following biomarkers in each of said samples:

(i) ratio of the number of ICOS FOXP3 double positive cells within a defined radius of influence around ICOS single positive cells to the total number of ICOS single positive cells,

(ii) mean distance between each ICOS single positive cell and its nearest ICOS FOXP3 double positive cell,

(iii) proportion of FOXP3 positive cells which are ICOS positive, and

(iv) density of ICOS positive cells,

pairing data for each of said one or more biomarkers with data for disease outcome or response to the anti-ICOS and/or anti-TReg immunotherapeutic agent in each patient, and

grouping data for each of said one or more biomarkers to identify a numerical cut-off which defines two groups of statistically significant data for disease outcome or response to the anti-ICOS and/or anti-TReg immunotherapeutic agent,

wherein said cut-off represents a reference value for classifying patients with the same type of cancerous solid tumours according to their predicted prognosis or predicted response to the anti-ICOS and/or anti-TReg immunotherapeutic agent.

Each of the biomarkers described here may be used individually or in combination with one or more other biomarkers. The etiology and progression of cancer are multi-factorial, and greater predictive value may be obtained using combinations of biomarkers. Statistical modelling methods such as logistic regression may be used to determine which biomarkers and combinations of biomarkers provide the best predictive value. Reference values for multiple biomarkers may be combined to provide a formula by which patients may be classified according to their prognosis and/or likelihood of response to treatment. Such a formula may be applied in determining whether a patient is likely to benefit from anti-ICOS and/or anti-TReg therapy and thus for identifying patients for treatment with an anti-ICOS and/or anti-TReg therapeutic agent, wherein a patient who meets or exceeds a reference value calculated according to the formula receives treatment with the anti-ICOS and/or anti-TReg therapeutic agent.

Embodiments of the invention will now be described in more detail, with reference to the accompanying drawings. Those skilled in the art will recognise, or will be able to ascertain using no more than routine experimentation, many equivalents to the specific embodiments of the invention described herein. Such equivalents are intended to be within the scope of protection of the appended claims.

### BRIEF DESCRIPTION OF THE DRAWINGS

FIG. 1 is a flow diagram of methods in which biomarkers are used in selecting patients for treatment with anti-ICOS cancer immunotherapy according to the present invention, showing embodiments of the process of data collection to assess the cancer immune contexture and predict the eligibility and need for anti-ICOS treatment in patients with a confirmed diagnosis of HCC. Patients for whom the biomarker reading is positive (“yes” arm) are administered anti-ICOS antibody KY1044, optionally in combination with other treatment. Patients for whom the biomarker reading is negative (“no” arm) are either not prescribed anti-ICOS therapy, or are given anti-ICOS therapy in combination with one or more other treatments (e.g., administration of other agents which will have the effect of influencing the biomarker and crossing the “yes/no” threshold).

FIG. 2 is a bar graph showing a significant increase in the density of ICOS FOXP3 double positive TReg in the TME vs the normal adjacent tissue (peritumour).

FIG. 3 is a bar graph showing a significant increase of the ratio of ICOS FOXP3 double positive TReg to total TReg (all FOXP3+) cells in the TME vs the normal adjacent tissue (peritumour).

FIG. 4 shows Kaplan-Meier curves of overall survival over time, separated according to the density of ICOS positive cells in the TME of HCC tumour biopsies (n=142 patients). The 142 HCC samples from the Taiwan NTU cohort were stratified according to the highest quartile (dotted line) of ICOS cell density per mm² vs the bottom 3 quartiles (solid line), representing a cut-off density of 120 cells per mm². Log-rank (Mantel-Cox) test p<0.05.

FIG. 5 shows Kaplan-Meier curves of overall survival over time, separated according to ICOS+FOXP3+ to total FOXP3+ cell ratio in the TME of HCC tumour biopsies (n=142 patients). A cut off ratio of 0.5 was used to separate the high (dotted line) vs the low (solid line) ratio population. Log-rank (Mantel-Cox) test p=0.0451.

FIG. 6 shows Kaplan-Meier curves of overall survival over time, separated according to according to the density of ICOS positive cells in the TME of HBV infected HCC tumour biopsies (n=87 patients). The 87 HCC samples from the Taiwan NTU cohort were stratified according to the highest quartile (dotted line) of ICOS cell density per mm² vs the bottom 3 quartiles (solid line), representing a cut-off of 100 cells per mm². Log-rank (Mantel-Cox) test p<0.05.

FIG. 7 shows Kaplan-Meier curves of overall survival over time, separated according to the density of ICOS positive cells in the TME of AJCC stage 2 HCC tumour samples (n=41 patients). The 41 AJCC stage 2 HCC samples from the Taiwan NTU cohort were stratified according to the highest quartile (dotted line) of ICOS cell density per mm² vs the bottom 3 quartiles (solid line), representing a cut-off of 100 cells per mm². Log-rank (Mantel-Cox) test p<0.01.

FIG. 8 shows Kaplan-Meier curves of relapse free survival rate (% RFS), separated according to the total density of ICOS positive cells in the TME in AJCC stage 2 HCC tumour biopsies (n=41 patients). The 41 AJCC stage 2 HCC samples from the Taiwan NTU cohort were stratified according to the highest quartile (dotted line) of ICOS cell density per mm² vs the bottom 3 quartiles (solid line), representing a cut-off of 100 cells per mm². Log-Rank (Mantel-Cox) test p<0.05.

FIG. 9 shows Kaplan-Meier curves of overall survival over time, separated according to the average distance of ICOS single positive cells to their nearest ICOS FOXP3 double positive cell in the TME. The 142 HCC samples from the Taiwan NTU cohort were stratified according to the median average distance: low distance (<105 μm for the solid line) vs high distance (≥105 μm dotted line). Log-Rank (Mantel-Cox) test p<0.05.

FIG. 10 shows Kaplan-Meier curves of overall survival overtime, separated according to the ratio of the number of ICOS FOXP3 double positive cells within a defined radius of influence around ICOS single positive cells to the total number of ICOS single positive cells in the TME of HCC. The 142 HCC samples from the Taiwan NTU cohort were stratified according to a value of an average of more or less than 0.1 double positive cells within 30 μm of ICOS single positive cells. Low incidence (<0.1 for the solid line) vs high incidence (0.1 dotted line). p<0.05 Log-Rank (Mantel-Cox).

FIG. 11 shows reduction in the ratio of ICOS FOXP3 double positive cells to total FOXP3 positive cells (representing the ICOS+ TReg proportion within all TReg) in human patients before and after administration of anti-ICOS monoclonal antibody KY1044. A) Cohort 1 (0.8 mg flat dose, Q3W) patient with leiomyosarcoma; B) cohort 2 (2.4 mg flat dose Q3W) patients with carcinoma of unknown primary (circles), renal cell carcinoma (triangles) and pancreatic cancer (squares); C) cohort 3 (8 mg flat dose Q3W) patient with metastatic prostate cancer; D) cohort 4 (24 mg flat dose Q3W) patients with pancreatic (circles) and oesophageal (triangles) cancer and E) cohort 5 (80 mg flat dose Q3W) patients with colon adenocarcinoma (circles), pancreatic (triangles) and mesothelioma (squares).

FIG. 12 (A) is a tissue section image on which a boundary defining the tumour core has been drawn, defining an area of approximately 2 cm×1 cm, which includes over half the tissue section. Non-tumour features are marked for exclusion from the tumour core area. Only tumour tissue that is within the marked boundaries is included for assessment as TME.

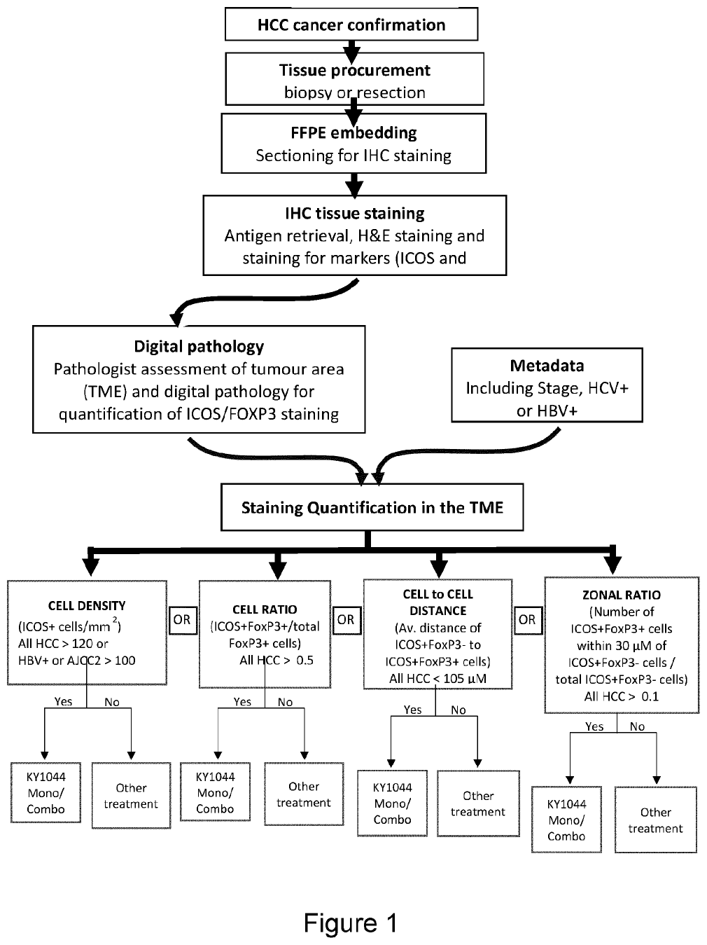

(B) is an enlargement of an area of (A). IHC staining is visible, including haematoxylin dye which stains all cell nuclei, and darker stains of two distinct chromogens for ICOS and FOXP3 respectively.

(C) is the identical view to (B), additionally showing square boxes representing ICOS FOXP3 double positive cells (identified by dual staining) and circles representing ICOS single positive cells (identified only by ICOS-specific staining, FOXP3 negative). “Nearest neighbour” lines connect each ICOS single positive cell to its nearest ICOS FOXP3 double positive cell. Not all ICOS FOXP3 double positive cells are connected, because some ICOS FOXP3 double positive cells in the tissue are not the nearest ICOS FOXP3 double positive cell to any ICOS single positive cell.

(D) is an enlargement of an area of (C). Distances are measured between every ICOS single positive cell and ICOS FOXP3 double positive cell in the TME. The shortest distance from each ICOS single positive cell to an ICOS FOXP3 double positive cell (i.e., the distance to the nearest ICOS FOXP3 double positive cell) is indicated by a connecting line. Individual distance measurements from a subset of ICOS single positive cells to their nearest ICOS FOXP3 double positive cell are shown, these being 50.7, 36.6, 45.4 and 39.5 μm, respectively—such measurements would be recorded for every ICOS single positive cell. These distances are measured using the Halo® software package.

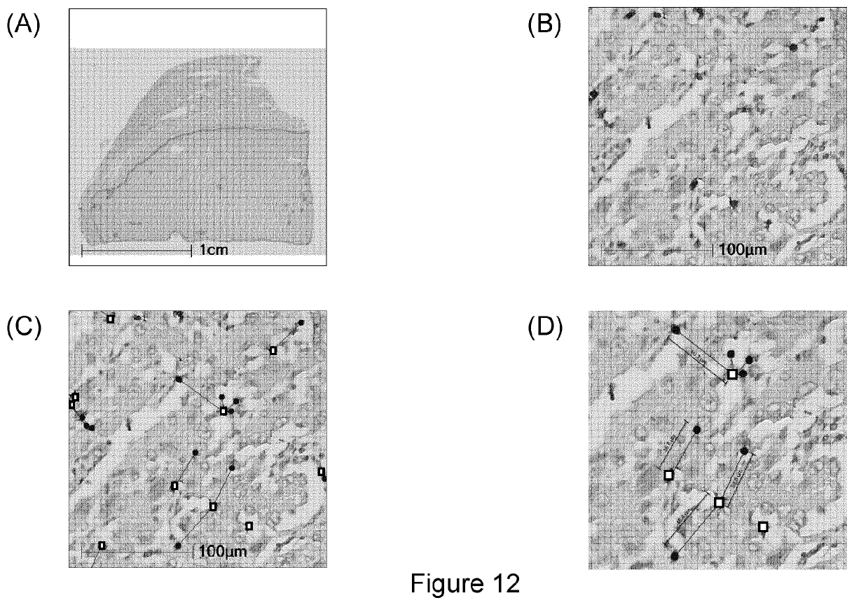

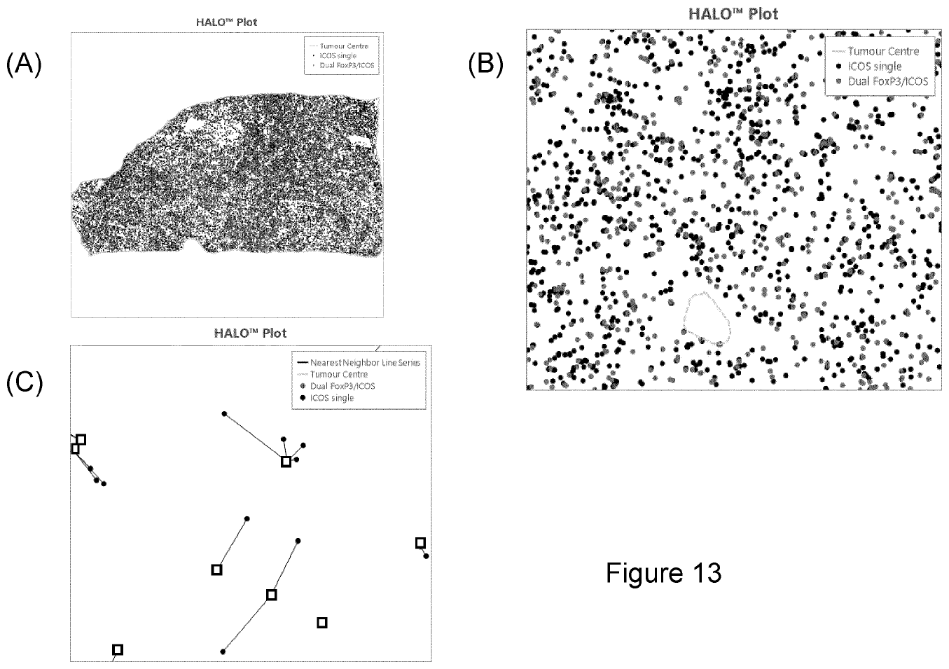

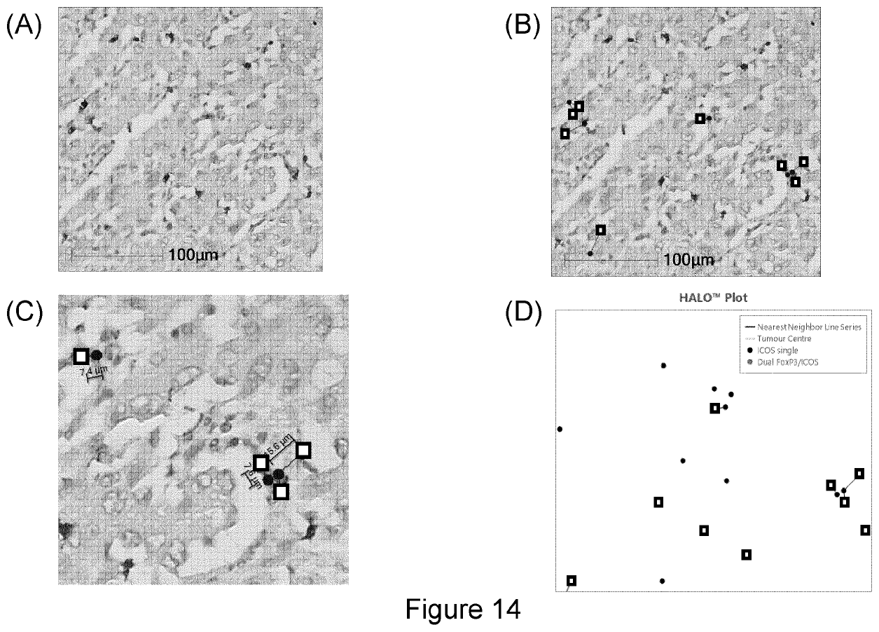

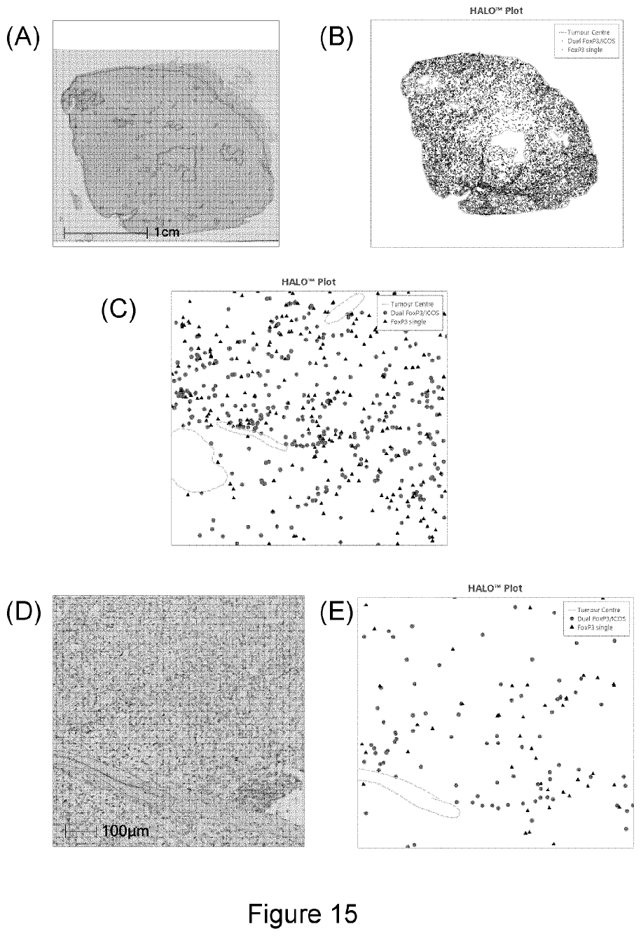

FIG. 13 (A) is a Halo® plot of the area of tumour core defined by the boundaries in FIG. 12 (A) with ICOS single positive (black) and ICOS FOXP3 double positive (grey) cells marked, without the underlying tissue image.

(B) is an enlargement of an area of (A).

(C) is an enlargement of an area of (B) and is a Halo® plot of the area shown in FIG. 12 (C). Nearest neighbour lines are shown from each ICOS single positive cell (circle) to its nearest ICOS FOXP3 double positive cell (square).

FIG. 14 (A) is an enlargement of an area of the tissue section depicted in FIG. 12(A), which area overlaps with the area shown in FIG. 12(B).

(B) is the identical view to (A), additionally showing circles representing ICOS single positive cells (identified only by ICOS-specific staining, FOXP3 negative) and square boxes representing ICOS FOXP3 double positive cells (identified by dual staining) that are within 30 μm of any ICOS single positive cell. Lines connect each ICOS single positive cell to ICOS FOXP3 double positive cells within 30 μm. ICOS FOXP3 double positive cells that are not within 30 μm of any ICOS single positive cell are not marked. ICOS single positive cells that are not within 30 μm of any ICOS FOXP3 double positive cell are not marked.

(C) is an enlargement of an area of (B). Example distance measurements are shown between ICOS single positive cells and ICOS FOXP3 double positive cells within 30 μm. These distances are 7.4 μm, 7.6 μm and 15.6 μm respectively. These distances would be measured from every ICOS single positive cell to every ICOS FOXP3 double positive cell within 30 μm of it, across the whole TME. These distances are measured using the Halo® software package.

(D) is a Halo® plot of a slightly zoomed in area corresponding to (B), without the underlying tissue image, and with all detected ICOS single positive cells (circles) and ICOS FOXP3 double positive cells (squares) marked. Lines connect each ICOS single positive cell to ICOS FOXP3 double positive cells within 30 μm.

FIG. 15 (A) is a tissue section image on which a boundary defining the tumour core has been drawn, defining an area of approximately 2 cm×2 cm, which includes most of the tissue section. Non-tumour features are marked for exclusion from the tumour core area. Only tumour tissue that is within the marked boundaries is included for assessment as TME.

(B) is a Halo® plot of the area of tumour core defined by the boundaries in (A) with ICOS FOXP3 double positive cells (grey circles) and FOXP3 single positive cells (black triangles) marked, without the underlying tissue image.

(C) is an enlargement of an area of (B).

(D) and (E) are a scanned image and a Halo® plot respectively of an area within (C). A tissue feature excluded from the TME area is ringed. IHC staining is visible in (D), including haematoxylin dye which stains all cell nuclei, and darker stains of two distinct chromagens for ICOS and FOXP3 respectively. (E) marks ICOS FOXP3 double positive cells as grey circles and FOXP3 single positive cells as black triangles, and the underlying tissue image is not shown.

### DETAILED DESCRIPTION

### Provision of Tumour Samples

To assess the cellular composition, immune contexture and specific spatial arrangement within the TME, a sample of the tumour tissue is provided in vitro. The sample may be obtained from a resected tumour after curative surgery (surgery to excise the tumour) or from a biopsy sample obtained from a tumour remaining in vivo. Resected tissue is preferred for ease of analysis in view of the greater volume of tissue generally available, but biopsy material (e.g., from core needle biopsy) may equally be used. Fine needle biopsy is an alternative, although the sample should comprise sufficient tumour core tissue for the analysis to be representative, so larger sampling (e.g., open biopsy) methods are preferred. Alternatively, where in vivo imaging systems permit assessment of biomarkers directly in situ within the TME, a surgical sampling step may be omitted. In general, however, methods of the invention are generally performed on samples in vitro.

In addition to cancerous cells which constitute the bulk of the tumour core, the TME includes a variety of immune cells, such as T lymphocytes of various sub-types, monocytes, neutrophils, dendritic cells and macrophages.

Assessment of protein expression on clinical biopsies is frequently carried out on formalin fixed paraffin embedded (FFPE) slides to assess tumour characteristics and expression of indicative prognostic markers. These can be used to help tailor the correct treatment for a given patient or retrospectively to distinguish between responder and non-responder patients for a given therapy through identification of new biomarkers.

Once a tissue sample has been obtained (e.g., from resected tissue or biopsy specimens), it will usually be treated to preserve its integrity and prepare it for testing. Tissue may be fixed in 10% formalin followed by embedding in paraffin, with a standard block shape being 0.5×1×1 cm. An alternative to this process is the fresh frozen procedure where tissues can be embedded in optimal cutting temperature (OCT) compound and flash frozen in dry ice before transferring to liquid nitrogen for long term storage. The FFPE process offers benefits of long term storage at room temperature while fresh frozen is a quicker process for storing tissue and keeps antigens in a native format but requires access to dry ice for preparing and shipment and liquid nitrogen for long term storage.

A two dimensional section of the tissue sample may be provided for testing. A minimum of one section is prepared per patient or per tumour. Optionally, multiple sections may be prepared, from the same tissue sample or from multiple samples, e.g., from different regions of one tumour or from multiple tumours. For FFPE samples, the blocks are loaded on to a microtome to be cut into sections, between 4-5 microns in diameter, and mounted onto a glass slide suitable for staining, usually a standard size of 25 mm×75 mm. Fresh frozen blocks are cut and mounted in similar way via a cryostat, which is essentially a microtome in a freezer, allowing sections to remain frozen during the process. Tissue sections are then presented for testing to determine biomarker readings, e.g., by immunohistochemistry (IHC) and digital image analysis.

The area of tumour core in a tissue section can be defined for assessment. Resected tissue will often comprise non-tumour tissue in addition to tumour tissue. This may be the case also for biopsied tissue, although a biopsy sample may simply consist of tumour core tissue. The extent of a tumour is typically determined by a pathologist, who can define the margin of the tumour in a sample of tissue. The region within the tumour (>500 μm inwards of the margin) is referred to as the tumour core and the tumour microenvironment refers to this area. The TME may be contrasted with the peritumour stroma, which is defined as tissue >500 μm outwards from the tumour margin and generally comprises normal, non-cancerous tissue, which may or may not be of the same tissue-type or organ from which the tumour is derived. A boundary representing the tumour margin may be drawn, either directly over a physical IHC slide (e.g., manually on glass) or preferably electronically as a visual overlay on a digital image of the tissue section. The tumour margin is typically a freeform shape, and in two dimensions on a tissue section it may be represented as a single loop or, e.g., where multiple patches of tumour/non-tumour tissue appear in the section, may comprise multiple loops. Annotation of a tissue section may also define boundaries around one or more areas to be excluded from the TME, such as artefacts or tissue folds. FIG. 12.

### Detection of ICOS

Sequences of human, mouse and cynomolgus ICOS are available from NCBI as human NCBI ID: NP_036224.1, mouse NCBI ID: NP_059508.2 and cynomolgus GenBank ID: EHH55098.1. Since patients in the present invention are preferably human patients, references herein to ICOS refer to or comprise human ICOS unless the context dictates otherwise.

ICOS is a marker of activated T cells. ICOS positive cells may be identified by detecting the ICOS receptor on the cell surface. An ICOS-binding agent such as an antibody, e.g., anti-ICOS clone D1K2T, is brought into contact with the tissue section under conditions allowing binding between the agent and ICOS, where present. The agent is either itself detectable (e.g., carries a detectable label) or is indirectly detectable by binding a labelled secondary agent specifically to the anti-ICOS agent (e.g., a secondary antibody which carries a detectable label). Using IHC, for example, the anti-ICOS antibody (e.g., D1K2T, which is a rabbit IgG) is bound to ICOS positive cells and is detected by a secondary antibody (e.g., antibody to rabbit IgG Fc) carrying a detectable label. Excess unbound agent is removed by washing, and the detectable label is then detected to locate ICOS in the tissue, thereby identifying the presence and position of ICOS positive cells.

An ICOS positive (ICOS+) cell is a cell which expresses ICOS as detectable using such methods as are outlined here. The ICOS positive cell may express other markers in addition to ICOS, such as FOXP3. An ICOS positive cell may be negative for FOXP3, in which case it may also be referred to as an ICOS single positive cell (particularly when ICOS is the sole antigen detected among a plurality of antigens stained for) or as an ICOS+ FOXP3− (ICOS positive FOXP3 negative) cell. An ICOS positive cell may be positive for FOXP3, in which case it may also be referred to as an ICOS FOXP3 double positive cell, or an ICOS+ FOXP3+ cell. Conversely, an ICOS negative (ICOS −) cell is a cell on which ICOS is not detectable using such methods as are outlined here (e.g., the cell exhibits no ICOS label significantly above background in IHC).

### Detection of FOXP3

FOXP3 is a marker of TReg. FOXP3 positive cells may be identified by detecting FOXP3 within the cells. FOXP3 is an intracellular molecule, so tissue samples should be treated to expose the antigen, e.g., by incubating with detergent. A FOXP3-binding agent such as an antibody, e.g., anti-FOXP3 clone 236A/E7, is brought into contact with the tissue section under conditions allowing binding between the agent and FOXP3, where present. The agent is either itself detectable (e.g., carries a detectable label) or is indirectly detectable by binding a labelled secondary agent specifically to the anti-FOXP3 agent (e.g., a secondary antibody which carries a detectable label). Using IHC, for example, the anti-FOXP3 antibody (e.g., clone 236A/E7) is bound to ICOS positive cells and is detected by a secondary antibody carrying a detectable label. Excess unbound agent is removed by washing, and the detectable label is then detected to locate FOXP3 in the tissue, thereby identifying the presence and position of FOXP3 positive cells.

A FOXP3 positive (FOXP3+) cell is a cell which expresses FOXP3 as detectable using such methods as are outlined here. The FOXP3 positive cell may express other markers in addition to FOXP3, such as ICOS. A FOXP3 positive cell may be negative for ICOS, in which case it may also be referred to as a FOXP3 single positive cell (particularly when FOXP3 is the sole antigen detected among a plurality of antigens stained for), or an ICOS− FOXP3+(ICOS negative FOXP3 positive) cell. A FOXP3 positive cell may be positive for ICOS, in which case it may also be referred to as an ICOS FOXP3 double positive cell, or an ICOS+ FOXP3+ cell. Conversely, a FOXP3 negative (FOXP3−) cell is a cell on which FOXP3 is not detectable using such methods as are outlined here (e.g., the cell exhibits no FOXP3 label significantly above background in IHC).

### Immunohistochemistry

Tissue sections may be stained to visualise antigens using IHC. In brief, IHC involves incubating the tissue section with an antigen-specific agent (e.g., antibody) to allow binding of the agent to its cognate antigen, washing excess unbound agent, and detecting the presence of bound agent, thereby visualising the presence and location of the antigen in the tissue. IHC can be performed directly using directly linked reporter antibodies or indirectly through antigen binding of primary antibody followed by linked or conjugated secondary antibodies. While the former can be more time efficient, the later can provide a more flexible approach with the benefit of signal amplification. IHC may be performed in multiplex on the same tissue section with multiple different antigen-specific agents in order to detect and locate multiple different antigens, using different signals/reporters/labels for the different antigens, e.g., fluorescent or enzyme multiplex IHC [29; 30; 31; 32; 33].

IHC is commonly used on sections of FFPE and fresh frozen tissue. To begin the IHC process on FFPE slides, the paraffin on the tissue is first removed through a series of incubations in xylene, ethanol and water. Due to the covering of antigen epitopes by fixative, FFPE slides are usually required to undergo a process called antigen retrieval, a process that breaks down the fixative methylene bridges to “uncover” the antigen of interest, allowing antibodies to bind. This can be achieved by two main methods: heat-induced epitope retrieval (HIER) and protease-induced epitope retrieval (PIER) [34; 35]. In HIER, the tissue mounted slide is boiled for several minutes before washing and is usually carried out with the use of a pressure cooker, a microwave, or a vegetable steamer while PIER is a process of incubating with a specific enzyme, such as trypsin or proteinase K, for several minutes at 37° C. before washing. Note, the exact time of each process needs to be optimised depending on the technique used and the antigen that is being retrieved for optimum retrieval and avoidance of damaging the tissue. In contrast, fresh frozen slides do not require an antigen retrieval step but will require a short fixation step if staining for intracellular targets such as FOXP3. This is usually carried out with alcohol, which unlike paraformaldehyde does not mask antigen epitopes.

For staining of intracellular targets such as FOXP3, FFPE and fixed fresh frozen slides are incubated in detergent, such as 0.25% Triton-X 100 in PBS, for 15-30 minutes at room temperature before washing. Tissues are then blocked to prevent non-specific binding of primary antibodies by incubating with blocking buffer for 1 hour at room temperature. Endogenous alkaline phosphatase (AP) in tissues can also remain prevalent in slides prepared from frozen tissue and requires blocking at this stage with levamisol. Once tissue samples are blocked, the antigen of interest can then be stained with primary antigen-specific antibodies (anti-ICOS and anti-FOXP3). Note, antigen specific antibodies need to be confirmed for use in IHC staining as they need to bind target following the processes outlined above. The primary antibodies must be specific for a given epitope on the target of interest and must not bind to other non-relevant targets to avoid background staining or false positive staining. The most convincing evidence for an antibody specificity is a lack of binding in knockout tissues or cells for the antigen in question. Excess unbound antibody is removed by washing. For chromogenic IHC, slides should next be blocked for peroxidase activity by incubating in hydrogen peroxide in methanol. If primary antibody is not directly labelled, reporter conjugated secondary antibody is then incubated and following final wash steps the reporter detected.

The use of different reporters can provide different advantages and disadvantages. Chromogenic IHC makes use of enzyme linked moieties that catalyse the conversion of substrates into insoluble, coloured precipitates, which allows the visualisation of a given antigen [29; 30]. The staining is permanent, unlikely to degrade over time and can be used in parallel with histological dyes such as haematoxylin and eosin. However, only low numbers of antigens can be assessed due to the low number of enzymes and enzyme substrates available commercially. Immunofluorescence (IF) IHC, uses antigen specific antibodies linked to fluorescent moieties, which allows for an increased number of staining markers to be used in parallel. Disadvantages to this method include signal deterioration over time and spectra overlap between fluorophores when multiple antigens are assessed [31; 33]. For the purposes of the present invention, both forms of IHC are more than adequate for staining for ICOS and FOXP3. As an example of the staining process using chromogenic IHC, tissues should be stained for both ICOS and FOXP3 using two primary antibodies of different species, for example mouse and sheep IgG, followed by two secondary antibodies targeting the two distinct species of primary antibody, an anti-mouse and anti-sheep, conjugated to horse radish peroxidase (HRP) or alkaline phosphatase (AP). The expression of ICOS and FOXP3 can then be revealed by applying two different dyes that react with HRP or AP, for example DAB and BCIP/NBT, staining the tissue brown and blue/black respectively.

Serial staining immunofluorescence, such as chipcytometry, is a process able to quantitatively analyse multiple antigens via staining with antigen specific antibodies linked to fluorescent moieties on FFPE or fresh frozen tissues. Fluorescence can be assessed with high dynamic range imaging combined with artificial intelligence software to generate analysis of cell types in a section [36]. As explained before, fresh frozen sections do not require antigen retrieval and therefore produce better quality results over FFPE tissue using this method. However, the chip cytometry technique is still possible on FFPE slides with the addition of an antigen retrieval step similar to uncover antigens required for this analysis. Following this, the tissue of both FFPE and fresh derived tissues can then be re-fixed by the standard process into the cytometry chips. The tissues can then be stained for up to five separate antigens in a process similar to that described above for standard IHC using five directly labelled primary antibodies. For example, two primary antibodies targeting ICOS and FOXP3 directly conjugated to two distinct fluorophores, for example APC and GFP, will allow distinction of ICOS and FOXP3 antigen expression in the tissues upon analysis. Spatial arrangements in the tissue can then be assessed, preferably assisted by software. One main distinction between this technique and standard IHC is that the fixing process stabilises the tissue allowing storage for up to 2 years at room temperature. Additionally, this allows the tissue to be bleached in order to quench the fluorescence antibodies without suffering damage, permitting tissue to be re-stained for additional five antigens. Through a process of repeat staining and bleaching, an almost unlimited protein biomarkers can be stained on the same slide with high definition and low background fluorescence.

Mass Cytometry Imaging (MCI) is an alternative to the standard enzyme or fluorescent IHC methods described above. Imaging Mass Cytometry (IMC) and Multiplexed Ion Beam Imaging (MIBI), are two forms of novel Mass Cytometry Imaging that can provide extremely detailed expression of multiple antigens from both FFPE and fresh frozen samples in a given area [37; 38]. In IMC, tissues are stained with metal-tagged, antigen-specific primary antibodies, the expression of each antigen can then be assessed by successively ablating very small areas of tissue (e.g., 1 μM²) with a concentrated laser beam and analysing the proportions of the specific metal ions via detecting their specific mass using Cytometry Time-Of-Flight (CyTOF). Alternatively, using MIBI, the process is very similar with the exception that an oxygen duoplasmatron primary ion beam is used to rasterize the tissue, successively ablating a thin layer and releasing the metal isotopes as ions [39; 40; 41; 42; 43]. In both ways, the amount of antigen specific antibodies in each section can be assessed via quantifying the specific masses of metal tags down to a resolution of 1 Da and a highly detailed, digital image of the whole tissue is then reconstructed using the information acquired. As an example, following a staining process similar to that outlined for standard IHC and using antigen retrieval if required, the tissues are stained with anti-ICOS and anti-FOXP3 primary antibodies linked to rare-earth, heavy-metal isotypes, such as ¹⁰²Pd and ²⁰⁹Bi (many other heavy metal isotypes would be possible) [44]. Expression of ICOS and FOXP3 in the tissue would then be progressively analysed using the process outlined above. Due to the resolution of this technique, it may be used to assess any of the tissue parameters and biomarkers described herein. The ability to distinguish isotypes down to 1 Da allows MCI to analyse up to 40 markers in parallel while avoiding the issues of spectral overlap. Therefore, multiple cell types can be assessed simultaneously on the same slide. Additionally, the technique is not limited by signal decay or background fluorescence.

### Digital Image Analysis

Tissue samples provided as sections may be viewed by microscopy. Brightfield illumination is suitable for visualisation of contrasting dyes from IHC. A tissue sample may be scanned to provide a digital image of the tissue (e.g., a magnified, brightfield image). Image magnification, e.g., 20× or 40×, facilitates analysis. The image may be annotated to define the area of tumour core, as described. In the work described herein, we scanned 5 μm IHC stained tissue slides at 20× magnification using a Hamamatsu Nanozoomer digital scanner in brightfield mode.

Cells may be counted and intercellular distances measured in order to take biomarker readings from the defined area of tumour core. Preferably, cell counting and distance measurement is automated using software analysis of a digital image. We used the HALO® platform (Indica Labs) to perform digital image analysis. In brief, the process involved a 3-chromogen colour deconvolution to separate the IHC chromogens for ICOS and FOXP3 respectively and the nuclear counterstain. Cell objects were formed by applying weighted optical density values for the individual chromogens. Each positive cell type was then identified using defined size, shape and subcellular compartment staining parameters. Background staining, which did not correspond to cell labelling, was identified and subtracted, so that the presence of ICOS or FOXP3 was indicated by an above-background level of staining. A classifier was developed and integrated into the algorithm to automatically segment the tissue regions of interest for analysis. The completed algorithm was applied to all tissue section images in the study in an automated and objective manner to generate detailed cell-by-cell data.

### Cell Density

The density of a type of cell in the TME is the total number of those cells divided by the area of the TME, and is typically expressed as number of cells per mm². The cell type of interest can be identified on a tissue section (or image thereof) through detection of markers such as ICOS and FOXP3 as detailed elsewhere herein, allowing cells to be counted. As described, counting may be automated using software to count all objects in a specified field of view which meet defined criteria, thus one may instruct software to count all ICOS positive cells in the tumour core. Where other cell markers besides ICOS are detected, this cell count may comprise multiple distinct populations, e.g., ICOS single positive cells and ICOS FOXP3 double positive cells. The total number of ICOS positive cells is all cells on which ICOS is detected at a level above background, thus including ICOS single positive cells as well as cells on which ICOS is present in addition to other detected markers, including ICOS FOXP3 double positive cells. The area of tumour core (TME) may also be determined using automated software, guiding the software by drawing a boundary around the tumour core as described herein. The number of ICOS positive cells in the TME is divided by the area of said TME to give the density.

An increased density of ICOS positive cells in the TME is a biomarker for response to anti-ICOS immunotherapeutic agents, which may also deplete or inhibit TReg, such as the anti-ICOS antibody KY1044. Examples 2, 3, 4 and 8 illustrate the determination and use of this biomarker.

### (ICOS+FOXP3+)/Total FOXP3+ Proportion

This proportion or ratio is calculated by determining the number of ICOS FOXP3 double positive cells in the TME, determining the number of FOXP3 positive cells in the TME, and dividing the former by the latter. The cell type of interest can be identified on a tissue section (or image thereof) through detection of markers (ICOS, FOXP3) as detailed elsewhere herein, allowing cells to be counted. As described, counting may be automated using software to count all objects in a specified field of view which meet defined criteria, thus one may instruct software to count all FOXP3 positive cells in the tumour core. Where other cell markers besides FOXP3 are detected, this cell count may comprise multiple distinct populations, e.g., FOXP3 single positive cells and ICOS FOXP3 double positive cells. The total number of FOXP3 positive cells is all cells on which FOXP3 is detected at a level above background, thus including FOXP3 single positive cells as well as cells on which FOXP3 is present in addition to other detected markers, including ICOS FOXP3 double positive cells. The area of tumour core (TME) may also be determined using automated software, guiding the software by drawing a boundary around the tumour core as described herein. The number of ICOS FOXP3 double positive cells in the TME is divided by the number of FOXP3 positive cells in the TME to give the ratio.

Since FOXP3 is a marker of TReg, this ratio may be referred to as the ICOS+ TReg proportion. A high proportion of TReg being ICOS+ is a biomarker indicative of an immunosuppressed TME. The presence of this biomarker indicates that an immunotherapeutic agent may benefit the patient. In one embodiment, a cut-off of 50% (ratio 0.5) is used to categorise patients. Thus, for example, an HCC patient is selected for anti-ICOS and/or anti-TReg treatment if testing of a tumour sample from the patient indicates that more than 50% of the FOXP3+(TReg) cells in the TME are ICOS+. Examples 1, 2, 8 and 9 illustrate the determination and use of this biomarker.

### Intercellular Proximity

Having identified the locations of cell types by detection of markers as described, distances between those cells can be measured. Measurements should be taken from nuclear centre to nuclear centre, which generally represents the middle of the cell in T cells, which have a compact rounded shape in the TME.

The mean distance between each ICOS single positive cell and its nearest ICOS FOXP3 double positive cell is determined by measuring the shortest distance from an ICOS single positive cell to an ICOS FOXP3 double positive cell, repeating this for every ICOS single positive cell, and dividing the sum of these shortest distances by the number of ICOS single positive cells. As with other biomarkers described herein, measurements are taken in the tumour core (TME) area of the tissue sample (e.g., on a digital image of a tissue section).

Proximity between ICOS FOXP3 double positive cells and ICOS single positive cells represents the proximity of strongly immunosuppressive cells to TEff. Close proximity (short distance) is a biomarker indicative of an immunosuppressed TME. The presence of this biomarker indicates that an immunotherapeutic agent may benefit the patient. Examples 5 and 8 illustrate the determination and use of this biomarker.

### Zonal Influence

We define the zonal influence ratio to be the ratio of the number of ICOS FOXP3 double positive cells within a defined radius of influence around ICOS single positive cells to the total number of ICOS single positive cells.

The ratio may be found by identifying all ICOS FOXP3 double positive cells in the TME, identifying all ICOS single positive (i.e., ICOS+ FOXP3−) cells in the TME, then selecting the subset of ICOS FOXP3 double positive cells which are within the defined radius of any (one or more) ICOS single positive cells in the TME, and dividing the number in this subset by the number of ICOS single positive cells.

An ICOS FOXP3 double positive cell is counted if it is within the defined radius of any ICOS single positive cell. It is not counted if it is not within the defined radius of any ICOS single positive cell. An ICOS FOXP3 double positive cell which is within the defined radius of multiple ICOS single positive cells is counted only once. This subset of counted cells thus comprises all ICOS FOXP3 double positive cells which are within the defined radius of influence of any ICOS single positive cell in the TME.

The counted number of ICOS FOXP3 double positive cells is then divided by the number of ICOS single positive cells in the TME. The count of ICOS single positive cells is a total count including all ICOS single positive cells in the TME, whether or not these cells have an ICOS FOXP3 double positive cell within their defined radius.

This calculation provides the zonal influence ratio. A radius of 30 μm is preferred. Lymphocyte cell size varies between 7-15 μm in diameter. The immediate extracellular environment influences the cell, and where two cells are within approximately 30 μm one may expect communication via soluble intercellular mediators and potentially via direct cell:cell contact. Immunosuppressive TReg such as ICOS FOXP3 double positive cells can suppress FOXP3 negative lymphocytes through direct interaction or through release of immunosuppressive cytokines such as IL10 and TGFβ. Thus, to identify cells which are within a zone of influence of one another, we define a radius of 30 μm around the cells of interest, in this case the ICOS+ FOXP3− cells. The invention is not limited by the precise distance of this radius, and wider (e.g., 50 μm) or narrower (e.g., 20 μm) zones may be drawn, for example within approximately 50% (15-45 μm) or 20% (24-30 μm) of the suggested 30 μm value. Examples 6 and 8 illustrate the determination and use of the zonal influence biomarker.

### Cancerous Solid Tumours

Cancers are typically classified by the type of tissue in which the cancer originates (histological type) or by primary site, the location in the body where the cancer first developed. Cancer classification is the subject of an international standard, ICD-O-3, produced by the World Health Organisation [45], incorporated by reference herein. The morphology axis provides five-digit codes ranging from M-8000/0 to M-9989/3. The first four digits indicate the specific histological term. The fifth digit after the slash (/) is the behaviour code, which indicates whether a tumour is malignant, benign, in situ, or uncertain (whether benign or malignant). A separate one-digit code is also provided for histologic grading (differentiation).

When considering cohorts of patients and reference values for biomarkers calculated therefrom, it is preferred that the reference value is calculated from patients with tumours of the same type. The tumours may be of the same physiological category (e.g., all carcinomas, all sarcomas or all myelomas, optionally including mixed type tumours spanning more than one category), and preferably the same tissue type, optionally a morphological type as classified by the ICD-O-3 (e.g., HCC M-8170/3). The tumour type may be for example liver cancer, e.g., HCC (e.g., ICD-O-3 M-8170/3, M-8171/3, M-8172/3, M-8173/3, M-8174/3 or M-8175/3), renal cell cancer (optionally renal cell carcinoma, e.g., clear cell renal cell carcinoma), head and neck cancer, melanoma (optionally malignant melanoma), non-small cell lung cancer (e.g., adenocarcinoma), bladder cancer, ovarian cancer, cervical cancer, gastric cancer, pancreatic cancer, breast cancer or testicular germ cell carcinoma, including the metastases of any solid tumour such as those listed.

It has been found that patients are less likely to respond to systemic anti-PD-1 immunotherapy if their tumour has metastasised to the liver. Patients with liver metastases are often excluded from clinical trials. Lee et al [46] suggested that antigen-specific TReg are activated in the liver and that these “liver-trained” TReg then suppress the wider anti-tumour immune response in the patient. Pre-clinical studies indicated that, for patients with liver metastasis, the sensitivity of a tumour to immune checkpoint inhibitors like anti-PD-1 might be improved by depleting TReg.

This rationale may extend to HCC which metastasises to other body tissues such as bone and lungs. Here the primary tumour is in the liver and the metastases are remote, but the effect described by Lee et al may occur, viz. tumour antigen-specific TReg are activated in the liver and propagate an immunosuppressive effect which reduces the sensitivity of tumours in other tissues to treatment with an immune checkpoint inhibitor.

In embodiments of the invention, a tumour may thus be:
a primary tumour of the liver (e.g., HCC);
a liver metastasis, i.e., a tumour of non-liver origin which originated elsewhere in the body and has metastasised to the liver; or
a tumour at a site other than the liver, wherein the patient also has a tumour metastasis in the liver.

In embodiments of the invention, the patient is one who has a tumour in the liver, which is optionally either a primary tumour of the liver (e.g., HCC) or a liver metastasis.

The present invention may involve identifying patients with such tumours as being suitable for treatment with anti-TReg immunotherapy, where such treatment also comprises administration of an immune checkpoint inhibitor (e.g., anti-PD-1 or anti-PD-L1).

### Hepatocellular Cancer (HCC)

Primary liver cancer is presently the fourth largest cause of cancer mortality globally, and most liver cancer cases are HCC. The carcinogenesis of HCC is attributed to chronic inflammation of the liver, predominantly from infection by hepatitis B virus (HBV) or hepatitis C virus (HCV) or from liver cirrhosis.

An elevated level of alpha-foetoprotein (AFP) in the blood is a known indicator of the presence of either a primary liver cancer or germ cell tumour. This protein is normally produced by the foetus and is undetectable in the blood of healthy adult males and non-pregnant healthy women. AFP level has been found to negatively correlate with survival in HCC patients.

The current treatment selected for HCC is commonly influenced by the disease stage. The AJCC staging system is a classification system developed by the American Joint Committee on Cancer for describing the extent of disease progression in cancer patients [28]. For patients with early-stage localised HCC, curative-intent treatment modalities include resection, ablation, and liver transplantation, whereas for patients with intermediate-stage localised HCC, image-guided transcatheter tumour therapies such as transarterial chemoembolisation have obtained survival benefits. However, the majority of patients with HCC either progress to or develop de novo locally advanced or metastatic diseases and are indicated for systemic therapy [47].

Sorafenib, a multikinase inhibitor, was first systemic therapy approved for HCC [48, 49]. Since 2016, 4 other targeted agents, including 3 multikinase inhibitors and one antivascular endothelial growth factor receptor (VEGFR) monoclonal antibody, have been demonstrated in phase Ill clinical trials to provide survival benefits to patients with advanced HCC [50, 51, 52, 53]. Consequently, regulatory agencies of multiple countries have approved lenvatinib as a first-line systemic therapy for HCC and approved regorafenib, cabozantinib, and ramucirumab (limited to patients with α-fetoprotein >400 ng/mL) for the treatment of those with HCC who have been previously treated with sorafenib. Anti-PD-1 antibody has also been used as a second line treatment in advanced HCC.

Patients in accordance with the present invention may have HCC which is associated with HBV and/or HCV infection, or which is not associated with HBV and/or HCV infection. The HCC may be of any stage (as determined according to AJCC staging manual, 8th edition), e.g., stage 1, stage 2 or stage 3. Patients may be male or female, adult or paediatric (<18 years). Patients may have undergone, or may be intended to undergo, hepatectomy for the tumour. Patients may have received previous treatment for the HCC, e.g., treatment with sorafenib and/or any other agent mentioned above. Patients may or may not have received previous treatment for the cancer by immunotherapy.

### Prognosis

Overall survival (OS) is defined as defined as the length of time from either the date of diagnosis or the start of treatment for a disease, such as cancer, until death. When used to describe populations of patients diagnosed with the disease (rather than individual patients), OS is typically expressed as a median, representing the length of time from either the date of diagnosis or the start of treatment that half of the patients in the group are still alive. In the HCC patient cohort described herein, OS was calculated from the date of hepatectomy to the date of the patient's death or their final follow-up day.

Recurrence free survival (RFS) in cancer refers to the length of time after primary treatment for the cancer ends that the patient survives without any signs or symptoms of that cancer. Also called disease free survival (DFS), relapse free survival.

Progression free survival (PFS) refers to the length of time during and after treatment that a patient lives with the disease but it does not get worse.

Time to progression (TTP) refers to the length of time from the date of diagnosis or the start of treatment until the disease starts to get worse or spread to other parts of the body.

In general, data for the same one or more measures of survival are obtained for all patients within a cohort (e.g., in a population of patients from which reference values are generated), in order to provide data for prognosis of survival by the same one or more measures.

### Immunotherapeutic Agents

An immunotherapeutic agent (agent used for immunotherapy) according to the present invention may be an anti-ICOS agent and/or it may be an anti-TReg immunotherapeutic agent. An anti-ICOS agent is an agent which binds ICOS, preferably the ICOS extracellular domain. An anti-TReg immunotherapeutic agent is one which depletes or inhibits TReg, preferably selectively, i.e., without depleting or inhibiting other T cells such as TEff or wherein the effect on such other T cells is less than on TReg. An agent which binds ICOS and also depletes or inhibits TReg is an anti-ICOS and anti-TReg immunotherapeutic agent. Examples include the anti-ICOS antibody KY1044 and other anti-ICOS antibodies with Fc effector function (e.g., anti-ICOS human IgG1).

Anti-TReg immunotherapies include antibodies, other biological agents, small molecules and cell therapies. The anti-TReg immunotherapeutic agent may be an antibody which binds TReg and mediates cellular effector functions (e.g., via an effector positive Fc region) to deplete or inhibit TReg. The anti-TReg immunotherapeutic agent may bind a marker of TReg, which is selectively or differentially expressed on TReg, e.g., on the TReg cell surface. Examples of such surface markers include ICOS, CD25, CCR8, CTLA-4 and glucocorticoid-induced TNF-related protein (GITR). MHC displayed epitopes may also represent such markers and offer an opportunity to target TReg via intracellular proteins such as FOXP3, which does not comprise an extracellular portion. Digested peptides of FOXP3 and other intracellular markers are presented on the TReg surface on MHC class I, whereupon they may be recognised by an agent that specifically binds the epitope displayed in the MHC groove. Dao et al described a TCR mimic antibody that binds epitope of intracellular FOXP3 [54]. As summarised by Dao et al, other TReg immunotherapeutic agents which have been described include targeting agents for CD25 (e.g., the anti-CD25 antibody daclizumab), agents for IL-2 receptor (e.g., IL-2 toxin fusions, such as dennileukin difitox which is a fusion protein of IL-2 and diphtheria toxin), anti-GITR antibodies which deplete TReg, anti-CTLA-4 antibodies which suppress TReg function and agents which disrupt tumour homing by TReg and/or modulate T cell plasticity. Antibody to chemokine receptor-4 (CCR4) was shown to selectively deplete TReg expressing a higher level of FOXP3, which resulted in augmentation of CD8+ T cell response specific for the NY-ESO-1 peptide. CCR4 is expressed on activated T cells, T helper 2, NK cells, macrophages, and dendritic cells, therefore confounding the selective effects. A defucosylated, humanised anti-CCR4 mAb, mogamulizumab, has been in clinical trials in various cancers. Chemokine receptor-8 (CCR8) is preferentially expressed on TReg in breast cancer patients and is associated with poor prognosis. CCR8 is also expressed on tissue-resident memory CD8+ T cells and NK-T cells, and therefore, the therapeutic potential of targeting this molecule remains to be investigated. In addition, modulating transforming growth factor (TGF)-beta, a crucial cytokine for TReg function has also been tried. Cyclophosphamide has been shown to suppress TReg.

Recently, anti-TReg therapies comprising anti-CD36 and/or PPARbeta inhibitors were described in WO2020053833. It disclosed a method of reducing the number of intratumoural TReg (e.g., CD4+ cells) in a subject, including for example administering a CD36 inhibitor. In some embodiments, the method included administering a PPARbeta inhibitor.

### Antibodies

Preferred immunotherapeutic agents are antibodies, which may be whole immunoglobulins or antigen-binding fragments thereof comprising immunoglobulin domains, whether natural or partly or wholly synthetically produced. Antibodies may be IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria. Antibodies can be humanised using routine technology.

The antibody comprises an antibody antigen-binding site (paratope) which binds to and is complementary to the epitope of its target antigen. The term “epitope” refers to a region of an antigen that is bound by an antibody. Epitopes may be defined as structural or functional.

Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Examples of antibody fragments include:

(i) a Fab fragment, a monovalent fragment consisting of the VL, VH, CL and CH1 domains; (ii) a F(ab′)2 fragment, a bivalent fragment including two Fab fragments linked by a disulfide bridge at the hinge region;

(iii) an Fd fragment consisting of the VH and CH1 domains;

(iv) an Fv fragment consisting of the VL and VH domains of a single arm of an antibody,

(v) a dAb fragment (Ward et al., (1989) Nature 341:544-546; which is incorporated by reference herein in its entirety), which consists of a VH or VL domain; and

(vi) an isolated complementarity determining region (CDR) that retains specific antigen-binding functionality.

Further examples of antibodies are H2 antibodies that comprise a dimer of a heavy chain (5′-VH-(optional hinge)-CH2-CH3-3′) and are devoid of a light chain.

Single-chain antibodies (e.g., scFv) are a commonly used fragment. Multispecific antibodies may be formed from antibody fragments. An antibody of the invention may employ any such format, as appropriate.

Digestion of whole immunoglobulins with the enzyme papain results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognised by Fc receptors (FcR) found on certain types of cells. Digestion of antibodies with the enzyme pepsin, results in the F(ab′)2 fragment in which the two arms of the antibody molecule remain linked and comprise two-antigen binding sites. The F(ab′)2 fragment has the ability to crosslink antigen.

A mAb² comprises a VH and VL domain from an intact antibody, fused to a modified constant region, which has been engineered to form an antigen-binding site, known as an “Fcab”. The technology behind the Fcab/mAb² format is described in more detail in WO2008/003103, and the description of the mAb² format is incorporated herein by reference. Further descriptions of this format can be found in WO2006/072620, WO2008/003116, WO2009/000006 and WO2009/0132876.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognise and bind antigen, although at a lower affinity than the entire binding site.

In a Fab, an antibody antigen-binding site may be provided by one or more antibody variable domains. In an example, the antibody binding site is provided by a single variable domain, e.g., a heavy chain variable domain (VH domain) or a light chain variable domain (VL domain). In another example, the binding site comprises a VH/VL pair or two or more of such pairs. Thus, an antibody antigen-binding site may comprise a VH and a VL.

Optionally, antibody immunoglobulin domains may be fused or conjugated to additional polypeptide sequences and/or to labels, tags, toxins or other molecules. Antibody immunoglobulin domains may be fused or conjugated to one or more different antigen binding regions, providing a molecule that is able to bind a second antigen. An antibody of the present invention may be a multispecific antibody, e.g., a bispecific antibody, comprising (i) an antibody antigen binding site for ICOS and (ii) a further antigen binding site (optionally an antibody antigen binding site, as described herein) which recognises another antigen.

The antibody variable domains include amino acid sequences of complementarity determining regions (CDRs; i.e., CDR1, CDR2, and CDR3), and framework regions (FRs). Thus, within each of the VH and VL domains are CDRs and FRs. The term “hypervariable region”, “CDR region” or “CDR” refers to the regions of an antibody variable domain which are hypervariable in sequence and/or form structurally defined loops. Generally, antigen binding sites of an antibody include six hypervariable regions: three in the VH (HCDR1, HCDR2, HCDR3), and three in the VL (LCDR1, LCDR2, LCDR3). These regions of the heavy and light chains of an antibody confer antigen-binding specificity to the antibody. A VH domain comprises a set of HCDRs, and a VL domain comprises a set of LCDRs. VH refers to the variable domain of the heavy chain. VL refers to the variable domain of the light chain. Each VH and VL is typically composed of three CDRs and four FRs, arranged from amino-terminus to carboxy-terminus in the following order: FR1, CDR1, FR2, CDR2, FR3, CDR3, FR4. An antibody may comprise an antibody VH domain comprising a VH CDR1, CDR2 and CDR3 and a framework. It may alternatively or also comprise an antibody VL domain comprising a VL CDR1, CDR2 and CDR3 and a framework. Examples of antibody VH and VL domains and CDRs according to the present invention are as listed in the appended sequence listing and tables that form part of the present disclosure. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. Unless otherwise stated, a “set of CDRs” includes HCDRs and LCDRs.

CDRs may be defined according to the Kabat system [55], the Chothia system [56] or the IMGT system [57, 58). IMGT is used by default, so CDRs and variable domain numbering herein are according to IMGT unless stated to the contrary.

Antibodies and other biological agents according to the present invention may be provided in isolated or purified form. An isolated antibody or protein is one that has been identified, separated and/or recovered from a component of its production environment (e.g., natural or recombinant). For example, the antibody or protein is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e., culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. In a preferred embodiment, antibodies of the invention are isolated or purified.

An antibody may comprise a VH domain that has at least 60, 70, 80, 85, 90, 95, 98 or 99% amino acid sequence identity with a VH domain of any of the antibodies described herein and/or shown in the appended sequence listing, and/or comprising a VL domain that has at least 60, 70, 80, 85, 90, 95, 98 or 99% amino acid sequence identity with a VL domain of any of those antibodies. Algorithms that can be used to calculate % identity of two amino acid sequences include e.g. BLAST, FASTA, or the Smith-Waterman algorithm, e.g. employing default parameters. Particular variants may include one or more amino acid sequence alterations (addition, deletion, substitution and/or insertion of an amino acid residue).

Alterations may be made in one or more framework regions and/or one or more CDRs. Variants are optionally provided by CDR mutagenesis. The alterations normally do not result in loss of function, so an antibody comprising a thus-altered amino acid sequence may retain an ability to bind the antigen (e.g., ICOS). It may retain the same quantitative binding ability as an antibody in which the alteration is not made, e.g. as measured in an assay described herein. The antibody comprising a thus-altered amino acid sequence may have an improved ability to bind the antigen.

Alteration may comprise replacing one or more amino acid residue with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences of the invention are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring.

The term “variant” as used herein refers to a peptide or nucleic acid that differs from a parent polypeptide or nucleic acid by one or more amino acid or nucleic acid deletions, substitutions or additions, yet retains one or more specific functions or biological activities of the parent molecule. Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide).

In some aspects, one can use “synthetic variants”, “recombinant variants”, or “chemically modified” polynucleotide variants or polypeptide variants isolated or generated using methods well known in the art. “Modified variants” can include conservative or non-conservative amino acid changes, as described below. Polynucleotide changes can result in amino acid substitutions, additions, deletions, fusions and truncations in the polypeptide encoded by the reference sequence. Some aspects use include insertion variants, deletion variants or substituted variants with substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties (e.g., acidic, basic, positively or negatively charged, polar or nonpolar, etc.). Conservative amino acid substitutions include replacement of a leucine with an isoleucine or valine, an aspartate with a glutamate, or a threonine with a serine. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W) [59]. In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

One can select the amino acid that will substitute an existing amino acid based on the location of the existing amino acid, including its exposure to solvents (i.e., if the amino acid is exposed to solvents or is present on the outer surface of the peptide or polypeptide as compared to internally localized amino acids not exposed to solvents). Selection of such conservative amino acid substitutions are well known in the art [60, 61, 62]. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

Antibodies disclosed herein may be modified to increase or decrease serum half-life, for example by sequence engineering of one or more antibody constant regions and/or fusion to other molecules, e.g., PEGylation or by binding to albumin, e.g. incorporating an albumin binding single domain antibody (dAb). Various half-life extending fusions have been described [63].

An antibody of the invention may be a human antibody or a chimaeric antibody comprising human variable regions and non-human (e.g., mouse) constant regions. The antibody of the invention for example has human variable regions, and optionally also has human constant regions.

Thus, antibodies optionally include constant regions or parts thereof, e.g., human antibody constant regions or parts thereof. For example, a VL domain may be attached at its C-terminal end to an antibody light chain kappa or lambda constant domain. Similarly, an antibody VH domain may be attached at its C-terminal end to all or part (e.g. a CH1 domain or Fc region) of an immunoglobulin heavy chain constant region derived from any antibody isotype, e.g. IgG, IgA, IgE and IgM and any of the isotype sub-classes, such as IgG1 or IgG4. Examples of human antibody constant domains are shown in Table C.

Constant regions of antibodies of the invention may alternatively be non-human constant regions. For example, when antibodies are generated in transgenic animals (examples of which are described elsewhere herein), chimaeric antibodies may be produced comprising human variable regions and non-human (host animal) constant regions. Some transgenic animals generate fully human antibodies. Others have been engineered to generate antibodies comprising chimaeric heavy chains and fully human light chains. Where antibodies comprise one or more non-human constant regions, these may be replaced with human constant regions to provide antibodies more suitable for administration to humans as therapeutic compositions, as their immunogenicity is thereby reduced.

### Fc Effector Functions, ADCC, ADCP and CDC

As discussed above, antibodies can be provided in various isotypes and with different constant regions. Examples of human IgG antibody heavy chain constant region sequences are shown in Table C. The Fc region of the antibody primarily determines its effector function in terms of Fc binding, antibody-dependent cell-mediated cytotoxicity (ADCC) activity, complement dependent cytotoxicity (CDC) activity and antibody-dependent cell phagocytosis (ADCP) activity. These “cellular effector functions”, as distinct from effector T cell function, involve recruitment of cells bearing Fc receptors to the site of the target cells, resulting in killing of the antibody-bound cell. In addition to ADCC and CDC, the ADCP mechanism [64] represents a means of depleting antibody-bound T cells, and thus targeting high ICOS expressing TRegs for deletion.

Cellular effector functions ADCC, ADCP and/or CDC may also be exhibited by antibodies lacking Fc regions. Antibodies may comprise multiple different antigen-binding sites, one directed to ICOS and another directed to a target molecule where engagement of that target molecule induces ADCC, ADCP and/or CDC, e.g., an antibody comprising two scFv regions joined by a linker, where one scFv can engage an effector cell.

An antibody according to the present invention may be one that exhibits ADCC, ADCP and/or CDC. Alternatively, an antibody according to the present invention may lack ADCC, ADCP and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

A suitable antibody format for some therapeutic applications employs a wild-type human IgG1 constant region. A constant region may be an effector-enabled IgG1 constant region, optionally having ADCC and/or CDC and/or ADCP activity. A suitable wild type human IgG1 contant region sequence is IGHG1*01. Further examples of human IgG1 constant regions are shown herein.

A constant region may be engineered for enhanced ADCC and/or CDC and/or ADCP. The potency of Fc-mediated effects may be enhanced by engineering the Fc domain by various established techniques. Such methods increase the affinity for certain Fc-receptors, thus creating potential diverse profiles of activation enhancement. This can achieved by modification of one or several amino acid residues [65]. Human IgG1 constant regions containing specific mutations or altered glycosylation on residue Asn297 (e.g., N297Q, EU index numbering) have been shown to enhance binding to Fc receptors. Example mutations are one or more of the residues selected from 239, 332 and 330 for human IgG1 constant regions (or the equivalent positions in other IgG isotypes). An antibody may thus comprise a human IgG1 constant region having one or more mutations independently selected from N297Q, S239D, 1332E and A330L (EU index numbering). A triple mutation (M252Y/S254T/T256E) may be used to enhance binding to FcRn, and other mutations affecting FcRn binding are discussed in Table 2 of [66], any of which may be employed in the present invention.

Increased affinity for Fc receptors can also be achieved by altering the natural glycosylation profile of the Fc domain by, for example, generating under fucosylated or defucosylated variants [67]. Non-fucosylated antibodies harbour a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. An antibody according to the present invention may be fucosylated, afucosylated, defucosylated or under fucosylated.

To increase ADCC, residues in the hinge region can be altered to increase binding to Fc-gamma RIII [68]. Thus, an antibody may comprise a human IgG heavy chain constant region that is a variant of a wild-type human IgG heavy chain constant region, wherein the variant human IgG heavy chain constant region binds to human Fcγ receptors selected from the group consisting of FcyRIIB and FcyRIIA with higher affinity than the wild type human IgG heavy chain constant region binds to the human Fcγ receptors. The antibody may comprise a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. The variant human IgG heavy chain constant region can be a variant human IgG1, a variant human IgG2, or a variant human IgG4 heavy chain constant region. In one embodiment, the variant human IgG heavy chain constant region comprises one or more amino acid mutations selected from G236D, P238D, S239D, S267E, L328F, and L328E (EU index numbering system). In another embodiment, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system). The enhancement of CDC may be achieved by amino acid changes that increase affinity for C1q, the first component of the classic complement activation cascade [69]. Another approach is to create a chimeric Fc domain created from human IgG1 and human IgG3 segments that exploit the higher affinity of IgG3 for C1q [70]. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a constant region comprising one or more mutations selected from E345K, E430G, R344D and D356R, in particular a double mutation comprising R344D and D356R (EU index numbering system).

WO2008/137915 described anti-ICOS antibodies with modified Fc regions having enhanced effector function. The antibodies were reported to mediate enhanced ADCC activity as compared to the level of ADCC activity mediated by a parent antibody comprising the VH and VK domains and a wild type Fc region. Antibodies according to the present invention may employ such variant Fc regions having effector function as described therein.

ADCC activity of an antibody may be determined in an assay as described herein. ADCC activity of an anti-ICOS antibody may be determined in vitro using an ICOS positive T cell line as described herein. More generally, ADCC activity of an antibody may be determined in vitro in an ADCC assay using cells exhibiting cell surface display of the antigen recognised by the antibody.

For some patients it may be preferred to use antibodies without Fc effector function. Antibodies may be provided without a constant region, or without an Fc region—examples of such antibody formats are described elsewhere herein. Alternatively, an antibody may have a constant region which is effector null. An antibody may have a heavy chain constant region that does not bind Fcγ receptors, for example the constant region may comprise a Leu235Glu mutation (i.e., where the wild type leucine residue is mutated to a glutamic acid residue). Another optional mutation for a heavy chain constant region is Ser228Pro, which increases stability. A heavy chain constant region may be an IgG4 comprising both the Leu235Glu mutation and the Ser228Pro mutation. This “IgG4-PE” heavy chain constant region is effector null.

An alternative effector null human constant region is a disabled IgG1. A disabled IgG1 heavy chain constant region may contain alanine at position 235 and/or 237 (EU index numbering), e.g., it may be a IgG1*01 sequence comprising the L235A and/or G237A mutations (“LAGA”).

A variant human IgG heavy chain constant region may comprise one or more amino acid mutations that reduce the affinity of the IgG for human FcγRIIIA, human FcγRIIA, or human FcγRI. In one embodiment, the FcγRIIB is expressed on a cell selected from the group consisting of macrophages, monocytes, B-cells, dendritic cells, endothelial cells, and activated T-cells. In one embodiment, the variant human IgG heavy chain constant region comprises one or more of the following amino acid mutations G236A, S239D, F243L, T256A, K290A, R292P, S298A, Y300L, V3051, A330L, 1332E, E333A, K334A, A339T, and P396L (EU index numbering system). In one embodiment, the variant human IgG heavy chain constant region comprises a set of amino acid mutations selected from the group consisting of: S239D; T256A; K290A; S298A; 1332E; E333A; K334A; A339T; S239D and 1332E; S239D, A330L, and 1332E; S298A, E333A, and K334A; G236A, S239D, and 1332E; and F243L, R292P, Y300L, V3051, and P396L (EU index numbering system). In one embodiment, the variant human IgG heavy chain constant region comprises a S239D, A330L, or 1332E amino acid mutations (EU index numbering system). In one embodiment, the variant human IgG heavy chain constant region comprises an S239D and 1332E amino acid mutations (EU index numbering system). In one embodiment, the variant human IgG heavy chain constant region is a variant human IgG1 heavy chain constant region comprising the S239D and 1332E amino acid mutations (EU index numbering system).

An antibody may have a heavy chain constant region that binds one or more types of Fc receptor but does not induce cellular effector functions, i.e., does not mediate ADCC, CDC or ADCP activity. Such a constant region may be unable to bind the particular Fc receptor(s) responsible for triggering ADCC, CDC or ADCP activity.

### Anti-ICOS Antibodies

An anti-ICOS agent may be an antibody to ICOS, which binds to the ICOS extracellular domain and thereby targets T cells expressing ICOS.

Anti-ICOS antibodies have been reported to have anti-tumour effects resulting from binding ICOS+ T cells. An anti-ICOS antibody may provide an immunotherapeutic effect by various mechanisms. For example, anti-ICOS antibodies may have an agonistic effect on ICOS, thus enhancing the function of TEff cells, as indicated by an ability to increase IFNγ expression and secretion. Anti-ICOS antibodies may optionally be engineered to deplete cells to which they bind, which should have the effect of preferentially downregulating ICOS+ TReg, lifting the suppressive effect of these cells on the effector T cell response and thus promoting the effector T cell response overall. The anti-ICOS antibody KY1044 has an ideal functional profile for use as an immunotherapeutic agent in the present invention, although other anti-ICOS agents may alternatively be used.

An anti-ICOS antibody may be any of the following antibodies, may comprise the VH and VL domains of any of the following antibodies or may comprise the HCDRs and/or LCDRs of any of the following antibodies:

(a) KY1044

(b) anti-ICOS antibodies described in PCT/GB2017/052352 WO2018/029474 or U.S. Pat. No. 9,957,323, the contents of which are incorporated herein by reference (e.g., STIM001, STIM002, STIM002B, STIM003, STIM004, STIM005, STIM006, STIM007, STIM008 or STIM009)

(c) anti-ICOS antibodies described in PCT/GB2018/053701 WO2019/122884 the contents of which are incorporated herein by reference (e.g., STIM017, STIM020, STIM021, STIM022, STIM023, STIM039, STIM040, STIM041, STIM042, STIM043, STIM044, STIM050, STIM051, STIM052, STIM053, STIM054, STIM055, STIM056, STIM057, STIM058, STIM059, STIM060, STIM061, STIM062, STIM063, STIM064, STIM065 or STIM066)

(d) anti-ICOS/PD-L1 mAb² bispecific antibodies described in PCT/GB2018/053698 WO2019/122882

(e) vopratelimab

(f) anti-ICOS antibodies described in WO2016/154177 or US2016/0304610 (e.g., 37A10S713, 7F12, 37A10, 35A9, 36E10, 16G10, 37A10S714, 37A10S715, 37A10S716, 37A10S717, 37A10S718, 16G10S71, 16G10S72, 16G10S73, 16G10S83, 35A9S79, 35A9S710 or 35A9S89)

(g) anti-ICOS antibodies described in WO2016/120789 or US2016/0215059 (e.g., 422.2 H2L5)

(h) antibody C398.4 or a humanised antibody thereof as described in WO2018/187613 or US2018/0289790, e.g., ICOS.33 IgG1f S267E, ICOS.4, ICOS34 G1f, ICOS35 G1f, 17C4, 9D5, 3E8, 1D7a, 1D7b or 2644 (for sequences see WO2018187613 Table 35), e.g., the antibody BMS-986226 in NCT03251924

(i) antibody JMAb 136, “136”, or any other antibody described in WO2010/056804

(j) antibody 314-8, the antibody produced from hybridoma CNCM I-4180, or any other anti-ICOS antibody described in WO2012/131004, WO2014/033327 or US2015/0239978

(k) antibody Icos145-1, the antibody produced by hybridoma CNCM I-4179, or any other antibody described in WO2012/131004, U.S. Pat. No. 9,376,493 or US2016/0264666

(l) antibody MIC-944 (from hybridoma DSMZ 2645), 9F3 (DSMZ 2646) or any other anti-ICOS antibody described in WO99/15553, U.S. Pat. Nos. 7,259,247, 7,132,099, 7,125,551, 7,306,800, 7,722,872, WO05/103086, US8,318,905 or U.S. Pat. No. 8,916,155

(m) anti-ICOS antibodies described in WO98/3821, U.S. Pat. No. 7,932,358B2, US2002/156242, U.S. Pat. Nos. 7,030,225, 7,045,615, 7,279,560, 7,226,909, 7,196,175, 7,932,358, 8,389,690, WO02/070010, U.S. Pat. Nos. 7,438,905, 7,438,905, WO01/87981, U.S. Pat. Nos. 6,803,039, 7,166,283, 7,988,965, WO01/15732, U.S. Pat. No. 7,465,445 or U.S. Pat. No. 7,998,478 (e.g., JMAb-124, JMAb-126, JMAb-127, JMAb-128, JMAb-135, JMAb-136, JMAb-137, JMAb-138, JMAb-139, JMAb-140 or JMAb-141, e.g., JMAb136)

(n) anti-ICOS antibodies described in WO2014/08911

(o) anti-ICOS antibodies described in WO2012/174338

(p) anti-ICOS antibodies described in US2016/0145344

(q) anti-ICOS antibodies described in WO2011/020024, US2016/002336, US2016/024211 or U.S. Pat. No. 8,840,889

(r) anti-ICOS antibodies described in U.S. Pat. No. 8,497,244

(s) antibody clone ISA-3 (eBioscience), clone SP98 (Novus Biologicals), clone 1 G1, clone 3G4 (Abnova Corporation), clone 669222 (R&D Systems), clone TQ09 (Creative Diagnostics), clone 2C7 (Deng et al. Hybridoma Hybridomics 2004), clone ISA-3 (eBioscience) or clone 17G9 (McAdam et al. J Immunol 2000).

KY1044 is a human anti-ICOS subclass G1 kappa monoclonal antibody. It has been developed with the intention of having dual mechanisms of action, namely depletion of TReg and co-stimulation (agonism) of TEff cells [7]. Sequences of the antibody KY1044 are shown in Table K herein.

An anti-ICOS antibody may be one that competes for binding to human ICOS with an antibody (e.g., human IgG1, or an scFv) comprising the heavy and light chain CDRs of KY1044. An anti-ICOS antibody may bind ICOS with at least the same affinity as KY1044, or with an affinity within 50%, within 25% or within 10% of the affinity of KY1044 (e.g., as determined by surface plasmon resonance with chip-bound antigen or chip-bound IgG anti-ICOS antibody).

An anti-ICOS antibody in the present invention may comprise one or more CDRs of KY1044 (e.g., all 6 CDRs, or a set of HCDRs and/or LCDRs) or variants thereof as described herein.

The antibody may comprise an antibody VH domain comprising CDRs HCDR1, HCDR2 and HCDR3 and an antibody VL domain comprising CDRs LCDR1, LCDR2 and LCDR3, wherein the HCDR3 is the KY1044 HCDR3 or comprises that HCDR3 with 1, 2, 3, 4 or 5 amino acid alterations. The HCDR2 may be the HCDR2 of KY1044 or it may comprise that HCDR2 with 1, 2, 3, 4 or 5 amino acid alterations. The HCDR1 may be the HCDR1 of KY1044 or it may comprise that HCDR1 with 1, 2, 3, 4 or 5 amino acid alterations.

The antibody may comprise an antibody VL domain comprising CDRs HCDR1, HCDR2 and HCDR3 and an antibody VL domain comprising CDRs LCDR1, LCDR2 and LCDR3, wherein the LCDR3 is the LCDR3 of KY1044 or comprises that LCDR3 with 1, 2, 3, 4 or 5 amino acid alterations. The LCDR2 may be the KY1044 LCDR2 or it may comprise that LCDR2 with 1, 2, 3, 4 or 5 amino acid alterations. The LCDR1 may be the KY1044 LCDR1 or it may comprise that LCDR1 with 1, 2, 3, 4 or 5 amino acid alterations.

An antibody may comprise:

an antibody VH domain comprising HCDRs HCDR1, HCDR2 and HCDR3, and

an antibody VL domain comprising LCDRs LCDR1, LCDR2 and LCDR3,

wherein the HCDRs are those of KY1044 or comprise the KY1044 HCDRs with 1, 2, 3, 4 or 5 amino acid alterations; and/or

wherein the LCDRs are those of antibody KY1044 or comprise the KY1044 LCDRs with 1, 2, 3, 4 or 5 amino acid alterations.

An antibody may comprise a VH domain comprising a set of HCDRs HCDR1, HCDR2 and HCDR3, wherein

HCDR1 is the HCDR1 of KY1044 SEQ ID NO: 1,

HCDR2 is the HCDR2 of KY1044 SEQ ID NO: 2,

HCDR3 is the HCDR3 of KY1044 SEQ ID NO: 3,

or comprising that set of HCDRs with 1, 2, 3, 4, 5 or 6 amino acid alterations.

An antibody may comprise a VL domain comprising a set of LCDRs LCDR1, LCDR2 and LCDR3, wherein

LCDR1 is the LCDR1 of KY1044 SEQ ID NO: 8,

LCDR2 is the LCDR2 of KY1044 SEQ ID NO: 9,

LCDR3 is the LCDR3 of KY1044 SEQ ID NO: 10,

or comprising that set of LCDRs with 1, 2, 3 or 4 amino acid alterations.

Amino acid alterations (e.g., substitutions) may be at any residue position in the CDRs.

Preferably, the antibody comprises an ICOS binding site comprising the full set of 6 CDRs of KY1044.

An anti-ICOS antibody according to the invention may comprise an antibody VH domain which is the VH domain of KY1044 SEQ ID NO 5 or which has an amino acid sequence at least 90% identical to the KY1044 antibody VH domain sequence. The amino acid sequence identity may be at least 95%, at least 96%, at least 97%, at least 98% or at least 99%.

An anti-ICOS antibody according to the invention may comprise an antibody VL domain which is the VL domain of KY1044 SEQ ID NO: 12 or which has an amino acid sequence at least 90% identical to the KY1044 antibody VL domain sequence. The amino acid sequence identity may be at least 95%.

Preferably, the antibody comprises the KY1044 VH and VL domains.

As detailed elsewhere herein, antibodies may include constant regions, optionally human heavy and/or light chain constant regions. An exemplary isotype is IgG, e.g., human IgG1. An anti-ICOS antibody may comprise the KY1044 heavy chain SEQ ID NO: 7 and/or the KY1044 light chain SEQ ID NO: 14. KY1044 may be recombinantly produced as two heavy chains of 454 amino acid residues each, and two light chains of 215 amino acid residues each with inter and intra chain disulphide bonds that are typical of IgG1 antibodies.

### Immunotherapy

Immunotherapy comprises treatment with an immunotherapeutic agent, which may be an anti-ICOS agent and/or an anti-TReg agent. It may be administered to the patient as a pharmaceutical formulation, e.g., by intravenous or subcutaneous injection. In preferred embodiments, the anti-ICOS and/or anti-TReg immunotherapy comprises administration of an anti-ICOS antibody such as KY1044 to the patient.

Patients may also receive, concurrently, previously or subsequently, treatment according to the standard of care for their cancer, optionally including surgery. Sorafenib, a multikinase inhibitor, was the first systemic therapy approved for HCC. Since 2016, 4 other targeted agents, including 3 multikinase inhibitors and one antivascular endothelial growth factor receptor (VEGFR) monoclonal antibody, have been demonstrated in phase Ill clinical trials to provide survival benefits to patients with advanced HCC [71, 72, 73, 74]. Regulatory agencies of multiple countries have approved lenvatinib as a first-line systemic therapy for HCC and approved regorafenib, cabozantinib, and ramucirumab (limited to patients with α-fetoprotein >400 ng/mL) for the treatment of those with HCC who have been previously treated with sorafenib. Anti-PD-1 antibody has also been used as a second line treatment in advanced HCC.

Treatments which may be combined with, or substituted for, anti-ICOS and/or anti-TReg immunotherapy include those that induce immunological cell death, which is characterised by release of ATP and HMGB1 from the cell and exposure of calreticulin on the plasma membrane [75, 76]. Treatments that induce immunological cell death include radiation (e.g., ionising irradiation of cells using UVC light or γ rays), chemotherapeutic agents (e.g., oxaliplatin, anthracyclines such as doxorubicin, idarubicin or mitoxantrone, BK channel agonists such as phloretin or pimaric acid, bortezomib, cardiac glycosides, cyclophosphamide, GADD34/PP1 inhibitors with mitomycin, PDT with hypericin, polyinosinic-polycytidylic acid, 5-fluorouracil, gemcitabine, gefitnib, erlotinib, or thapsigargin with cisplatin) and antibodies to tumour-associated antigens. The tumour-associated antigen can be any antigen that is over-expressed by tumour cells relative to non-tumour cells of the same tissue, e.g., HER2, CD20, EGFR. Suitable antibodies include herceptin (anti-HER2), rituximab (anti-CD20), or cetuximab (anti-EGFR).

Other treatments which may be combined with anti-ICOS and/or anti-TReg immunotherapy, and methods of administering anti-ICOS treatments and/or combination therapy, are described in WO2018/029474 which is incorporated herein by reference in its entirety.

Methods of the present invention, including methods of determining biomarkers and methods in which patient samples are monitored for a signature of response comprising changes in one or more biomarkers, may be used to inform prescription of these or other treatments.

A patient who receives surgery to remove or reduce a tumour may receive immunotherapy before and/or after the surgery. Immunotherapy following surgery may treat remaining tumour tissue and/or other remaining primary tumours or metastases.

### Statistical Methods and Modelling

Kaplan-Meier curves and log rank tests are examples of univariate analysis. They describe the survival solely according to the selected factor under investigation. An alternative method is the Cox proportional hazards (COX-PH) regression analysis, which works for both quantitative predictor variables and for categorical variables. Furthermore, the Cox regression model extends survival analysis methods to assess simultaneously the effect of several risk factors on survival time.

As demonstrated in the appended Examples, reference (cut-off) values may be determined as the value at which patients can be differentiated with statistical significance with respect to the clinical measure (e.g., OS). Cut-off values may be determined empirically by testing a series of cut-off values to identify a value providing statistical significance (e.g., p<0.05 by log rank test), preferably the value with the highest statistical significance. Preferably, cut-off values are determined from the data by ROC analysis—see [77], incorporated herein by reference. Optionally, the cut-off value is the median reading of the biomarker, the top quartile reading of the biomarker or the bottom quartile reading of the biomarker.

Where reference values are calculated, it may be convenient to include a multiplication or division factor, in order to express the value at a desired order of magnitude. For example, if a cut-off value is determined to be 0.001, this may conveniently be expressed as 1 by including a multiplication factor of 1000. Biomarker readings from test samples are then multiplied by the same factor, for comparison against the reference value. Similarly, reference numbers and biomarkers may be transformed to generate other values on any desired scale (whether numerical, visual (e.g., a colour chart), auditory or other), and the biomarker readings may still validly be compared against said reference values provided that the same transformation is consistently performed. Thus, the invention need not be constrained by the manner in which biomarker data are presented. Rather, readings and reference values used in the invention will be those that are representative of the underlying data, so that (for example) a biomarker reference value representing a density of 100 ICOS positive cells per mm² tumour core may be expressed as “100 cells per mm²” or as “0”. In the latter instance a subtraction of 100 is applied, so a tumour sample from which a density of 110 cells per mm² is calculated is then converted to 10 for comparison against the 0 reference value.

An example of the use of logistic regression analysis to identify biomarker variables that can be used to guide prognosis and treatment of patients is found in the study by Kagamu et al [27]. That study identifies the status of CD4+ T cells in the peripheral blood of patients as biomarkers that can identify patients presenting early disease progression after nivolumab treatment, enabling classification of patients as non-responders or responders. The authors describe a formula based on the percentages of CD62^{low} CD4+ T cells and CD25+ FOXP3+ cells in peripheral blood of patients with non-small cell lung cancer to predict non-responders. The prediction formula was developed by using the discovery cohort data with a logistic regression model. The performance of the prediction formula was evaluated using the independent validation cohort data. Survival curves were estimated using the Kaplan-Meier method. All P values were two-sided, and p<0.05 was considered statistically significant. Tests for differences between two populations were performed using a Student t test. Multiple-group comparison was performed using one-way ANOVA with Tukey post hoc analysis.

This type of modelling can be generally applied to clinical data from cancer patients in order to identify biomarkers and combinations of biomarkers which allow meaningful classification of patients, e.g., according to predicted survival and/or likelihood of response to treatment. A number of software packages are available to run the statistical analyses and modelling, including SAS 9.4 (SAS Institute Inc.) and Prism 8 (GraphPad).

### EXAMPLES

Much information can be read from a tumour tissue section, and insight is gained about the nature of the TME by observing the spatial arrangement of cells, including the distribution, spread, clustering and proximity of various different cell types in relation to one another. Previously, the density of immune cells within 20 μm of a melanoma cell was reported to be associated with response to immune checkpoint blockers (Gide et al 2020). In the present invention, we reveal that the spatial arrangement of immune cells expressing ICOS and/or FOXP3 in the TME is a biomarker for disease prognosis and for response to immunotherapy. In the Examples below we describe analyses of ICOS positive, FOXP3 positive and ICOS FOXP3 double positive cells in the TME of HCC samples, characterising the TME in terms of the density, ratios, intercellular distances and numbers of these cells. We co-stained for ICOS and FOXP3 and measured the density of ICOS single positive, FOXP3 single positive and ICOS FOXP3 double positive cells per mm², the ratio of different stained cells (e.g., ratio of FOXP3 single positive cells to ICOS FOXP3 double positive cells), the distance between different stained cells (e.g., average distance of ICOS FOXP3 double positive cells to ICOS single positive cells) and the number of ICOS FOXP3 double positive cells in a region of influence (defined by a radius of 30 μM) around ICOS single positive cells.

We looked for associations between these measurements of the TME and clinical outcomes or clinical metadata. We describe how such measurements represent biomarkers for disease prognosis (including overall survival) and for response to anti-ICOS and/or anti-TReg immunotherapy.

ICOS FOXP3 double positive cells are identified as active TReg, characteristic of an immunosuppressive TME. HCC tumours were found to contain a high proportion of ICOS+ TRegs, implying a strongly immunosuppressive environment. Higher density of ICOS+ cells, a higher ratio of ICOS+ FOXP3+ dual positive cells to total FOXP3+ cells, a closer proximity between ICOS+ TRegs and other ICOS+ cells, and higher numbers of ICOS+ TReg in regions of influence were all associated with poorer overall survival.

We describe how anti-TReg anti-ICOS and/or immunotherapy may provide clinical benefit, including extended survival, in patients exhibiting these biomarkers in the TME. The anti-ICOS antibody KY1044 is an ideal candidate therapeutic agent because of its ability to target ICOS positive cells and to selectively deplete cells with high expression of ICOS, removing the highly immunosuppressive TReg and boosting the anti-tumour immune response.

### Example 1. Increased Number and Ratio of ICOS Positive TRegs in the TME Vs Peritumour

It was previously reported that in a cohort of 20 HCC patients there was an increase in ICOS FOXP3 double positive cells in the TME compared with adjacent normal tissue[3]. In the present study we assessed data from a large cohort of patients, which confirm and extend the previous findings. We found a significant increase of ICOS FOXP3 double positive cells in the TME compared with peritumour tissue in HCC samples. We also observed a significant increase in the ratio of ICOS positive TReg (ICOS FOXP3 double positive cells) to total TReg (FOXP3 positive cells) (p<0.001) in tumour core vs peritumour. A high density of tumour-infiltrating TReg is thought to be an unfavourable prognostic indicator of HCC. With the fact that ICOS+ FOXP3+ cells have been reported to be highly immunosuppressive through the production of both TGF-β and IL-10[78], our findings indicate that HCC tumours should strongly benefit from a strategy aimed at depleting ICOS FOXP3 double positive cells. Tumours with higher ratios of ICOS FOXP3 double positive cells to ICOS negative FOXP3 positive cells should benefit the most.

### Tumour Samples

Tumour tissues were originally collected from HCC patients who received hepatectomy at the National Taiwan University Hospital, Taipei, Taiwan. Formalin-fixed, paraffin-embedded tissue sections (5 μm thickness) were retrieved for the present study from archived tissues.

### Patient Data

A total of 142 patients (male:female=112:30, median age of 61.0 years) were enrolled in the study cohort. Among them, 87 (61.3%) had chronic hepatitis B virus (HBV) infection, 33 (23.2%), had chronic hepatitis C (HCV) infection, and 22 (15.5%) had no HBV/HCV infection. No patients were double positive for HBV and HCV. Patients with AFP<20 ng/mL or early stage disease had significantly improved median overall survival (OS). OS was calculated from the date of hepatectomy to the date of the patient's death or their final follow-up day. Median OS was calculated as the length of time after diagnosis at which half of the patients in the cohort were still on study. In this cohort, the median OS was of 100.3 months. The age, gender and viral etiology were not significantly associated with changes in OS.

### Immunohistochemistry

A sequential dual multiplex IHC assay was performed to detect ICOS and FOXP3 expressing cells in the 5 μm tissue sections as follows. Tissue slides were deparaffinised, rehydrated, and autoclaved in citrate buffer (pH 6.0) for 10 minutes using a decloaking chamber for antigen retrieval, followed by blocking with hydrogen peroxide and then a casein-containing blocking reagent (Background Sniper, BioCare Medical) to reduce non-specific background staining. The slides were then incubated with an anti-ICOS antibody (dilution 1:800; clone D1K2T; Cell Signaling) at 4° C. overnight. The MACH1 Universal HRP-Polymer detection kit (BioCare Medical) was applied, including a secondary antibody to detect D1K2T, and an automated DAB detection was carried out as per manufacturer's recommendations. DAB (3,3′-diaminobenzidine) is a derivative of benzene and provides a brown colour stain.

Blocking steps was repeated and the slides were then incubated with anti-FOXP3 antibody (dilution 1:800; clone 236A/E7; Abcam) at 4° C. overnight. The MACH1 Universal HRP-Polymer detection kit was applied, including a secondary antibody for detection, and Vina Green Chromogen Kit (BioCare Medical) was carried out as per manufacturer's recommendations. The Vina Green Chromagen provides a green colour stain, used here as a secondary staining for FOXP3. The slides were counterstained with haematoxylin and bluing reagent, which converts the initial soluble red colour of the haematoxylin within the nucleus to an insoluble blue colour. The alkaline pH of the bluing solution also causes the mordant dye-lake to reform in the tissue and become more permanent. The result is a stain of all cell nuclei, which shows the overall architecture of the tissue and provides a contextual background against which the two IHC chromagens can be viewed, facilitating subsequent analysis by the pathologist or computer. Finally, all slides were removed from the instrument, washed, dehydrated and mounted in a permanent mounting medium following standard IHC procedures.

### Digital Pathology

The 5 μm IHC stained tissue slides were scanned at 20× magnification using a Hamamatsu Nanozoomer digital scanner in brightfield mode. Dual-IHC analysis of whole slide images was performed with the Indica Labs HALO® platform. In brief, the process involved a 3-chromogen colour deconvolution to separate the IHC chromogens (×2) plus counterstain. Cell objects were formed by applying weighted optical density values for the individual chromogens. Each positive cell type was then identified using defined size, shape and subcellular compartment staining parameters. A classifier was developed and integrated into the algorithm to automatically segment the tissue regions of interest for analysis. The completed algorithm was applied to all tissue section images in the study in an automated and objective manner to generate detailed cell-by-cell data. All scanned images were reviewed and the following regions were defined and manually annotated:
invasive margin: edge of tumour as defined by pathologist
core (TME): tumour centre >500 μm inwards from the invasive margin
peritumour stroma: tissue around tumour >500 μm outwards from the invasive margin.

Tissue folds and/or staining artefacts were omitted from the analysis by manual exclusion.

### Results and Conclusions

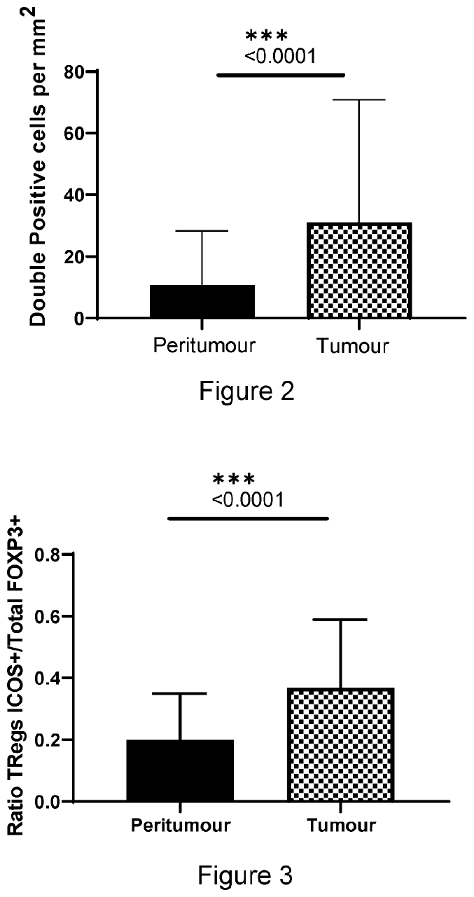

The density of ICOS+ FOXP3+ cells was significantly higher (p<0.001 by paired T test) in the TME (tumour core) than in the peritumour area. FIG. 2.

The ratio of ICOS positive TRegs (ICOS FOXP3 double positive cells) to total FOXP3 cells was also significantly higher in the TME (tumour core) than in the peritumour area (p<0.001 by paired T test). FIG. 3.

These findings are consistent with earlier work in HCC in which ICOS+ TRegs were more prevalent in TME than in the peritumour area [3].

The high number and proportion of TReg expressing ICOS in HCC implies a strongly immunosuppressive environment in these tumours. Anti-ICOS and/or anti-TReg immunotherapy is likely to be of benefit in HCC with these characteristics.

These effects were more pronounced in viral-related cancers, which suggests viral-associated or virally induced cancers (e.g., HBV positive HCC, HCV positive HCC) may be more likely to respond to anti-ICOS and/or anti-TReg immunotherapy, although the result did not reach statistical significance in the present study.

### Example 2. ICOS is a Biomarker for Disease Prognosis

Using the same HCC patient cohort as in Example 1, we uncovered links between patient survival and the density of ICOS+ cells and proportion of ICOS+ TReg in the tumour samples. Higher density of ICOS positive cells was linked to shorter OS. Higher proportion of ICOS positive TReg was also linked to shorter OS.

### Sample Processing

Tumour samples were processed and IHC and digital pathology work were conducted as described in Example 1.

### Data Analysis

Associations between cell measurements and survival were analysed by constructing Kaplan-Meier curves for patient populations divided according to different cell measurements. Differences in survival between patient populations were compared by log rank test. Kaplan-Meier log-rank analyses were performed using Graphpad Prism 8.3.1.

Statistical analyses were performed using SAS statistical software (version 9.4, SAS Institute Inc., Cary, N.C., USA). A two-sided p value of <0.05 was considered statistically significant. The OS and RFS of patients in different subgroups were estimated using the Kaplan-Meier method and compared using the log-rank test. The unpaired T test was used to compare the differences in cell densities.

The hazard ratio (HRs, Mantel-Haenszel) with 95% confidence intervals (CIs) was also used to estimate the degree of the association between the ratio of ICOS+ TReg to total TReg and prognosis of HCC cancer.

### Results and Conclusions

The effect of ICOS+ TReg proportion and of ICOS+ cell density and on HCC prognosis were each examined by constructing Kaplan-Meier curves, and differences in OS and were compared by log rank test. Patients with a high ratio of ICOS⁺FOXP3⁺/total FOXP3⁺ cells (p=0.074) or with high ICOS+ cell density in the TME were associated with a shorter OS (p<0.05).

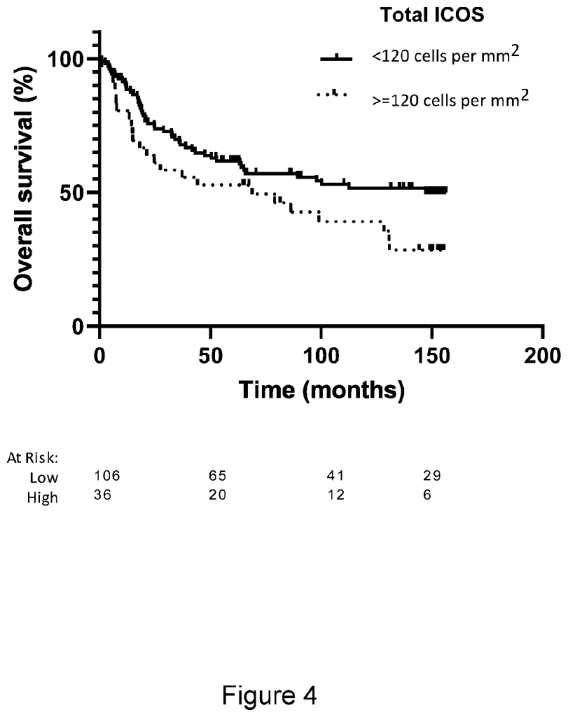

The results demonstrated a significant correlation between high ICOS cell density and bad prognosis (poor survival), suggesting that low ICOS expression, manifesting as a lower density of ICOS+ cells in the TME, is a predictor for better survival of patients with HCC. We found that for patients having 120 or more ICOS positive cells per mm² in the TME, median OS was more than halved compared with other patients. Patients with a low density (<120 cells per mm²) had a median survival of 147.3 months whereas those with a high density (>=120 cells per mm²) had a median survival of 68.9 months. FIG. 4. The cut-off of 120 cells is a rounded value. Statistical significance was observed with other similar cut-off numbers, e.g., 123 cells per mm². The hazard ratio (HRs, Mantel-Haenszel) with 95% confidence intervals (CIs) for the association between the density of ICOS+ cells and prognosis of HCC cancer was calculated as 0.5774 with CI 0.3346-0.9963; p<0.05

When the analysis was performed with figures for relapse free survival (RFS) rather than overall survival (OS), the log-rank (Mantel-Cox) test did not indicate statistical significance for the difference in % RFS for patient populations stratified according to the highest quartile (5120 cells per mm²) vs the bottom 3 quartiles (<120 cells per mm²).

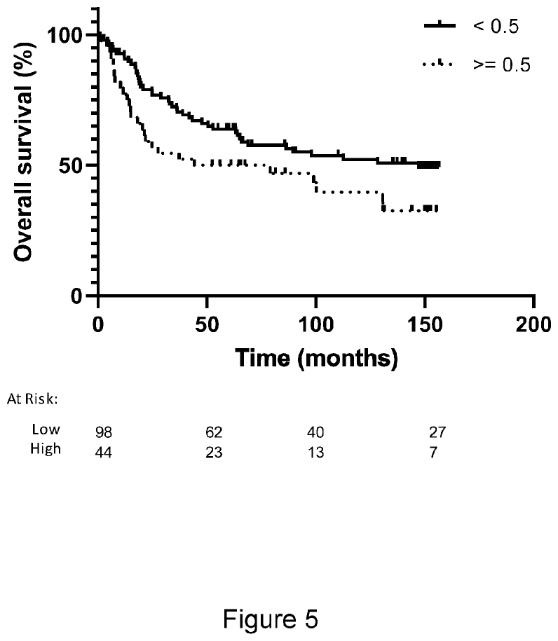

A high proportion of ICOS positive TRegs in the TReg population was also associated with worse prognosis for survival. We found that patients for whom 50% or more of TReg were ICOS positive, median OS was halved compared with other patients. Patients with a low ratio (<0.5) had a median survival of 147.3 months whereas those with a high ratio (>=0.5) had a median survival of 61.6 months. FIG. 5. Hazard ratio for the association was 0.5870 with confidence intervals of and CI 0.3486-0.9885; p=0.0451.

The cut-off ratio of 0.5 in this population was determined empirically as the ratio at which patients could be differentiated with statistical significance with respect to survival time. A split of the cohort using the median ratio (0.33) similarly divided patients into those with longer and shorter survival, and patients with a higher ratio had longer survival, but without meeting statistical significance. The same applied when splitting the cohort into patients with top quartile vs bottom quartile of the ratio, i.e., a difference was observed but did not meet statistical significance.

When the effect on RFS (rather than OS) was assessed, separating the cohort on a cut-off ratio of 0.5, we saw a clear separation of data, with % RFS being higher in patients with a ratio <0.50. This reflects the same trend observed with OS, but the difference for RFS was not statistically significant according to the log-rank (Mantel-Cox) test.

In conclusion, patients with a higher density of ICOS+ cells and/or a higher ratio of ICOS+ TReg to total TReg in the TME have a worse survival, and treatment with an anti-TReg therapeutic agent should address at least one cause of this by decreasing the number of ICOS+ TReg and effecting an anti-tumour response to improve survival.

### Example 3. ICOS is a Prognostic Biomarker in HBV Positive HCC

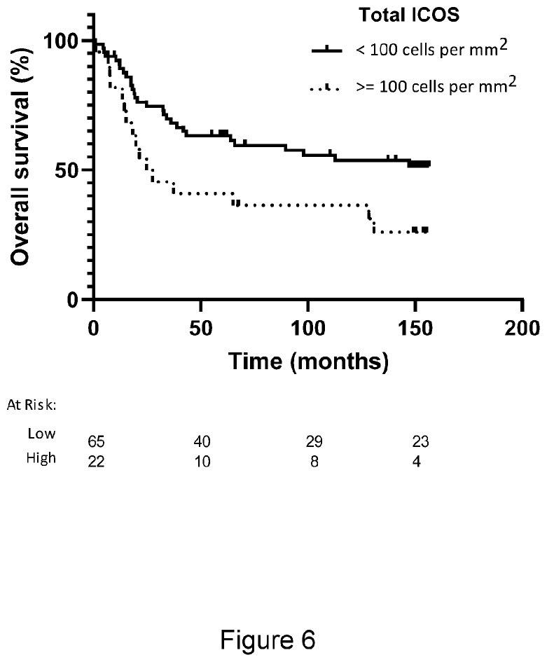

Chronic infection with hepatitis B virus (HBV) is linked to the pathogenesis of HCC. Through further analysis of the data from Example 2, we found that a high density of ICOS positive cells per mm² in the TME of HBV positive tumours was associated with poor prognosis. When focusing on HBV positive HCC tumours, the results demonstrated a significant correlation between high ICOS cell density and bad prognosis, suggesting that low ICOS expression, manifesting as a lower density of ICOS+ cells in the TME, is a predictor for better survival of patients with HCC associated with HBV infection.

Whereas a cut-off value of 120 cells per mm² was used to differentiate patient populations for HBV agnostic HCC (Example 2), a lower cut-off value of 100 cells per mm² was determined for HBV positive HCC. Patients with 100 or more ICOS positive cells per mm² or more in the TME (top quartile) exhibited a median OS of 26.1 months, whereas OS was undefined/not reached for patients having less than 100 ICOS cells per mm² (bottom three quartiles). FIG. 6. Statistical significance by log-rank (Mantel-Cox) test was p<0.05. The same analysis for RFS rather than OS did not meet statistical significance.

The hazard ratio (HRs, Mantel-Haenszel) with 95% confidence intervals (CIs) was also used to estimate the degree of the association between the density of ICOS positive cells and prognosis of HCC cancer (OS). HR of 0.4345 and CI 0.2131-0.8861 p=0.0219.

### Example 4. ICOS is a Prognostic Biomarker in AJCC Stage 2 HCC

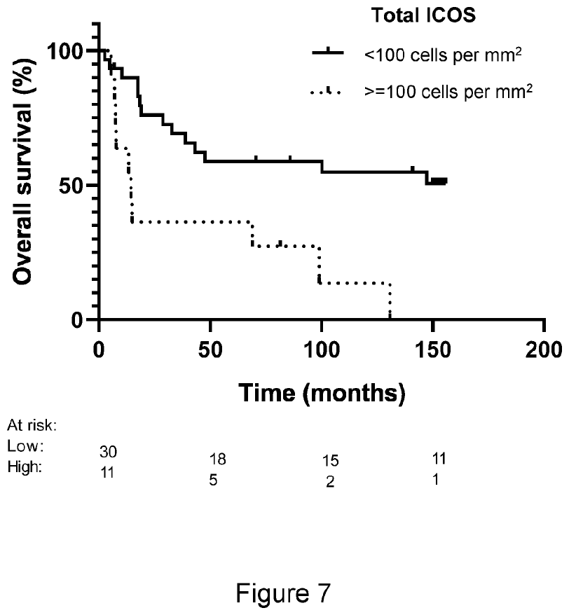

Through further analysis of the data from Example 2, we found that a high density of ICOS positive cells per mm² in the TME of AJCC stage 2 HCC tumours was associated with poor prognosis. Similarly to Example 3, we found that when focusing on AJCC2 HCC tumours, the results demonstrated a significant correlation between high ICOS cell density and bad prognosis, suggesting that low ICOS expression, manifesting as a lower density of ICOS+ cells in the TME, is a predictor for better survival of patients with AJCC2 HCC.

Whereas a cut-off value of 120 cells per mm² was used to differentiate patient populations for HBV agnostic HCC (Example 2), a lower cut-off value of 100 cells per mm² was determined for AJCC stage 2 HCC. Patients with 100 or more ICOS positive cells per mm² or more in the TME (top quartile) exhibited a lower median OS compared with patients having less than 100 ICOS cells per mm² (bottom three quartiles). FIG. 7. Statistical significance by log-rank (Mantel-Cox) test was p<0.01.

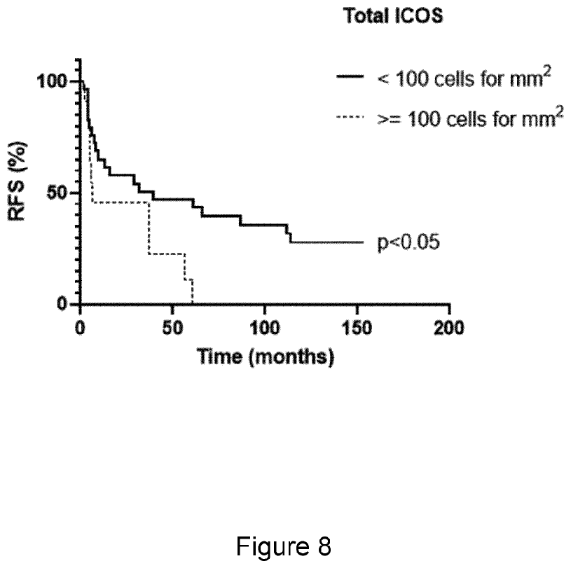

Patients with 100 or more ICOS positive cells per mm² or more in the TME (top quartile) also exhibited a lower % RFS compared with patients having less than 100 ICOS cells per mm² (bottom three quartiles). FIG. 8. Statistical significance by log-rank (Mantel-Cox) test was p<0.05.

We believe the lower cut-off value of 100 cells per mm² would also be appropriate for more advanced stages of HCC, e.g., stage 3 HCC. This could be verified by analysis of samples and survival data for the 18 patients with stage 3 AJCC in the present cohort.

### Example 5. Intercellular Distances are Biomarkers for Prognosis

The distribution of immune cells relative to each other may influence not only the anti-tumour immune status of a patient but also the patient's survival. Using the tumour samples from the same HCC patient cohort as the previous Examples, we measured distances between ICOS FOXP3 double positive cells and ICOS single positive (ICOS positive FOXP3 negative) cells. ICOS FOXP3 double positive cells represent active TReg which are highly immunosuppressive to CD8+ cytotoxic T cells and CD4+T helper cells. ICOS single positive cells are considered as non-TReg lymphocytes, such as activated CD8+ cytotoxic T cells and CD4+T helper cells. Thus in this study we investigated the spacing between the effector T cells which may be mediating an anti-tumour immune response, and active TReg which may be suppressing that response. Although intercellular distances were studied here in dead tissue, it is believed that the methods used to sample and preserve the tissue, and the IHC and digital pathology techniques used, preserve the integrity of the tissue such that the distances between cells are representative of those in the tumour in vivo immediately prior to sampling.

We discovered that the average distance from ICOS single positive cells to the nearest ICOS FOXP3 double positive cell was correlated with overall survival. A shorter distance between cells was associated with shorter survival.

### Cell:Cell Proximity Measurement

The cell density and distances between ICOS or FOXP3 single- and ICOS/FOXP3 dual-expressing cells in the tumour core, margin, and the peritumour area were quantitated by digital pathology as follows.

Quantification of cells positive for multiple chromogenic markers within a defined region was done. Quantification of cells in which multiple chromogenic markers co-localise was done.

Spatial relationships of the different cell populations identified were investigated. A classification algorithm was developed using a Random Forest approach and applied to each whole slide image to identify viable tissue within each region (margin/core/peritumour). A separate customised multiplex IHC algorithm was developed to quantify FOXP3 and ICOS single and dual ICOS/FOXP3 IHC positive stained cells. Specifically, haematoxylin and IHC chromogen stains were weighted differentially to segment cells within the regions and to establish an accurate cell count. Threshold values were adjusted for each chromogen to determine cells positive for FOXP3 only (teal nuclear chromogen), ICOS only (3,3′-diaminobenzidine (DAB) brown chromogen) and dual FOXP3/ICOS. FOXP3 and ICOS single and dual cell counts were normalised across the viable tissue area for each region to obtain the number of cells positive per mm² of tissue per region for each whole slide image.

The cell-based analysis data was further utilised to plot the spatial location of detected cell phenotypes within the tumour core. These plots were subsequently used to generate proximity and relative spatial distribution data for ICOS single and dual FOXP3/ICOS cells using the Spatial Analysis module embedded within Halo platform. For each set of sections per cancer type, average distance data for every ICOS single positive cell to its nearest dual FOXP3/ICOS positive cell within the tumour core per whole slide image were initially generated. This was followed by measuring the number of dual FOXP3/ICOS positive cells within 30 μm of every ICOS single positive cell within the tumour core per whole slide image. Proximity analysis was then performed for the dual FOXP3/ICOS positive cells within 30 μm of an ICOS single positive cell, to generate the average distance of these cells from their nearest ICOS single positive cell.

### Data Analysis

Data analysis was generally performed as described in Example 2.

### Results and Conclusions

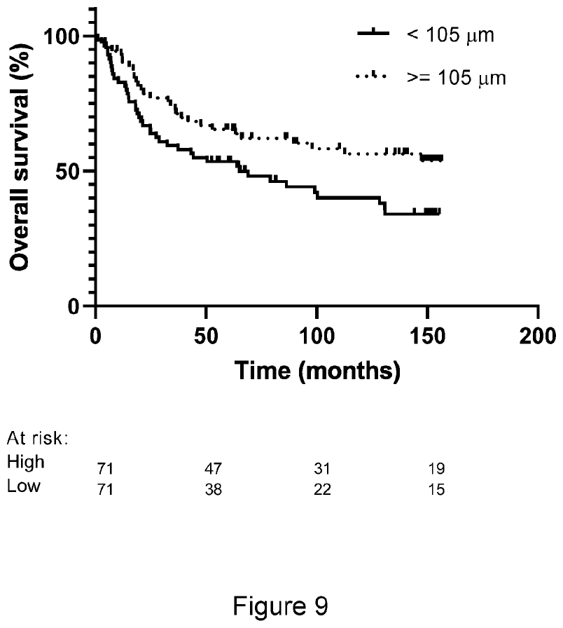

We calculated the average (mean) distance from ICOS single positive cells to their nearest ICOS FOXP3 double positive cell in each of the 142 HCC samples from the Taiwan NTU cohort. The median average distance in this cohort was 105 μm. Using this median as a cut-off value to stratify the cohort, we found that patients with an average distance of less than 105 μm had significantly lower overall survival than patients with an average distance of 105 μm or greater. FIG. 9. This stratification did not show a statistically significant association with RFS. The hazard ratio (HRs, Mantel-Haenszel) with 95% confidence intervals (CIs) was used to estimate the degree of the association between the distance of ICOS+ single positive cells to the nearest ICOS FOXP3 double positive cell and overall survival of HCC cancer. We calculated HR of 1.692 (high distance vs low distance) and CI were 1.063-2.694; p=0.0266.

Thus, a shorter distance between ICOS+FOXP3+ cells and ICOS+FOXP3− cells was significantly associated with a shorter OS (p<0.05), which suggests that ICOS+ TReg are actively immunosuppressing ICOS+ TEff in the TME of HCC.

### Example 6. Zonal Influence of Immunosuppressive TReg in Radius of TEff

Following on from the previous Examples, we further studied the spatial distribution of ICOS+ FOXP3+ and ICOS+ single positive cells to identify potential implication of having a high density of the highly immunosuppressive ICOS+FOXP3+ double positive TRegs next to ICOS+ single positive cells (potentially effector cells). Focusing on the TME, we identified and counted the number of ICOS FOXP3 double positive cells that were within 30 μm of any ICOS single positive cell.

Defining a zone by taking a radius of 30 μm around each ICOS single positive cell, we measured the total number of ICOS FOXP3 double positive cells falling within any one or more such zones in the TME, then divided this cell count by the total number of ICOS single positive cells in the TME area, to give a ratio which we term the zonal influence ratio.

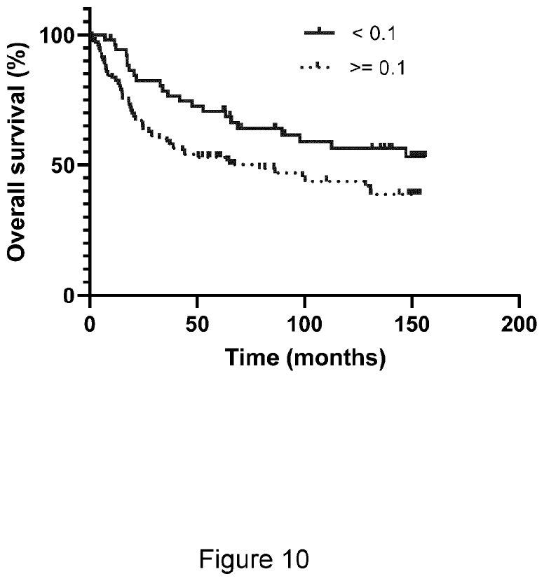

We identified a statistically significant association between overall survival and the zonal influence ratio. In patient samples having a zonal influence ratio of <0.1, the patient OS (but not RFS) was significantly greater than in samples having an average of ≥0.1. FIG. 10 and Table E6-1.

TABLE E6-1
Median survival

| | | |
|---|---|---|
| Bottom 3 quartiles | Undefined | |
| Top quartile | 79.00 | |
| Hazard Ratio (Mantel-Haenszel) | A/B | B/A |
| Ratio (and its reciprocal) | 0.6099 | 1.640 |
| 95% CI of ratio | 0.3819 to 0.9741 | 1.027 to 2.618 |

### Example 7. Value of Immunotherapy

The Examples above indicate that the following characteristics are all associated with a poorer OS:
greater ICOS expression (higher density of ICOS+ cells) in tumour core (TME)
greater ratio of ICOS*FOXP3+/total FOXP3+ cells
greater proximity (shorter distance) between ICOS+ TReg and other ICOS+ cells
greater number of ICOS+FOXP3+ TRegs within 30 m of ICOS single positive cells.

We conclude that an immunosuppressive TME is clinically significant in HCC. TReg could suppress function and proliferation of CD4+ and CD8+ TEff. This is consistent with earlier studies in which an increase of TReg and decrease of CD8+ T cells in TME was associated with poor prognosis in patients with solid tumours, including HCC [2].

Depleting/reducing ICOS^{high} positive TReg in such tumours, e.g., with an antibody such as KY1044, should improve the immune contexture by reducing immunosuppression and provide clinical benefit in patients with HCC and other tumours characterised by these features.

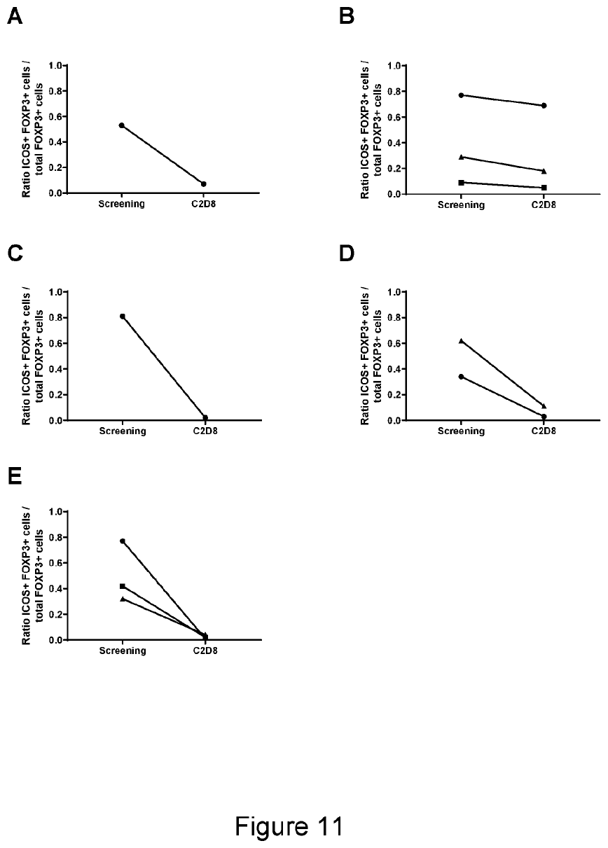

KY1044 has been shown to deplete ICOS+FOXP3+ TReg in cancer patients. In multiple cohorts of patients in a clinical trial with KY1044 monotherapy, KY1044 decreased the ratio of ICOS+FOXP3+ TReg to all FOXP3+ TReg from baseline (screening) to 8 days following the second cycle of dosing (C2D8) with dosing at 3 weekly intervals. FIG. 11.

### Example 8. Calculating Biomarkers from a Patient Tumour Sample

We illustrate the determination of biomarker readings from a tumour sample from the patient cohort described in Examples 1 to 6.

A tissue slide was prepared from the tumour sample, stained for ICOS and FOXP3, and digitally analysed as described in Example 1. FIGS. 12 to 14.

The area of tumour core (AREA_TME) was defined and quantified.

The following data were recorded for cells within the tumour core:
number of ICOS single positive cells (SPICOS_NB_TME);
number of FOXP3 positive cells (TOTFOXP_NB_TME);
number of ICOS FOXP3 double positive cells (DP_NB_TME);
the average (mean) distance from each ICOS single positive cell to its nearest ICOS FOXP3 double positive cell (DIST_SPICOS_TME), representing intercellular proximity between ICOS+ FOXP3+ cells and ICOS+ FOXP3− cells; calculation of this average was performed automatically by the Halo® software from its distance measurements and cell count data; and
the number of ICOS FOXP3 double positive cells that were within 30 μm of any ICOS single positive cell (DP_NB_30UMSPICOSROI_TME).

Summing the number of ICOS single positive cells and the number of ICOS FOXP3 double positive cells gives the total number of ICOS positive cells (TOTICOS_NB_TME).

Dividing the total number of ICOS positive cells by the area of tumour core gives the density of ICOS positive cells (TOTICOS_DEN_TME).

Dividing the number of ICOS FOXP3 double positive cells by the total number of FOXP3 positive cells gives the proportion of FOXP3 positive cells which are ICOS positive (“ICOS+ TReg proportion”) (RATIO_DP_TO_TOTFOXP_TME).

Dividing the number of ICOS FOXP3 double positive cells that were within 30 μm of any ICOS single positive cell by the total number of ICOS single positive cells in the TME gives the zonal influence ratio (RATIO_DP_30UMSPICOSROI_TO_SPICOS_TME).

TABLE E8-1
Data from tissue sample obtained from HCC stage 1 HBV+
tumour. Biomarker reference values calculated from the
whole patient cohort are shown in the far right column.

| | | Cut-off |
|---|---|---|
| | Sample | value |
| AREA_TME | 168.49 | |
| TOTICOS_NB_TME | 43347 | |
| TOTICOS_DEN_TME | 257 | 100 |
| SPICOS_NB_TME | 29254 | |
| TOTFOXP_NB_TME | 21825 | |
| DP_NB_TME | 14093 | |
| RATIO_DP_TO_TOTFOXP_TME | 0.646154 | 0.5 |
| DIST_SPICOS_DP_TME | 51.7492 | 105 |
| DP_NB_30UMSPICOSROI_TME | 6863 | |
| RATIO_DP_30UMSPICOSROI_TO_SPICOS_TME | 0.234600397 | 0.1 |

Each of the ICOS+ cell density, intercellular proximity, the ICOS+ TReg proportion, and the zonal influence ratio are biomarkers for prognosis and treatment of patients in the present invention. The biomarker readings for the patient can be compared against the reference values for the cohort. The example biomarker data from the sample would indicate a poor prognosis, because the density of ICOS positive cells is greater than 100 mm², the ratio of ICOS+ FOXP3+ cells to total FOXP3+ cells is greater than 0.5, the average distance from an ICOS+ FOXP3− cell to its nearest ICOS+ FOXP3+ cell is less than 105 μm, and the zonal influence ratio is greater than 0.1. A patient having an HCC tumour presenting with this data would be expected to have relatively short survival compared with other HCC patients. The biomarker data indicate that the patient would benefit from treatment with an anti-ICOS and/or anti-TReg immunotherapeutic agent, which could extend the patient's survival.

### Example 9. Calculating ICOS+ TReg Proportion

A tissue slide was prepared from another tumour sample from the patient cohort described in Examples 1 to 6. The section of tissue was stained for ICOS and FOXP3, and digitally analysed as described in Example 1. FIG. 15.

Analysis read-out for the ICOS+ TReg proportion was as follows:

Number of ICOS FOXP3 double positive cells in TME=11,066

Number of FOXP3 single positive cells in TME=10,842

Total FOXP3 cells in TME=21,908

Ratio=11,066/21,908=0.51

### REFERENCES

1 Wei, Duffy & Allison, Cancer Discov. 2018
2 Gao Q, et al. J Clin Oncol 25:2586-93 2007
3 Tu J F, et al. Sci Rep 6:35056 2016
4 Strauss L, et al J Immunol 180:2967-2980 2008
5 Kornete M, Sgouroudis E, Piccirillo C A. J Immunol 188:1064-1074 2012
6 32nd Annual Meeting and Pre-Conference Programs of the Society for Immunotherapy of Cancer (SITC 2017): Part Two. Poster P288 Journal for ImmunoTherapy of Cancer 5:87 2017
7 Sainson et al., BioRxiv September 2019 doi:10.1101/771493
8 Mo et al Vaccine 35:5932-5938 2017
9 Hansen A, et al. Annals of Oncology 29 2018
10 El-Khoueiry A B, et al. Lancet 389:2492-2502 2017
11 Zhu A X, et al. Lancet Oncol 19:940-952 2018
12 Yau T, et al. Annals of Oncology 30 2019
13 Finn R S, et al. J Clin Oncol 1901307 2019
14 Calderaro, J. et al. Programmed death ligand 1 expression in hepatocellular carcinoma: Relationship With clinical and pathological features. Hepatology 64, 2038-2046 2016
15 Yau T, et al. J Clin Oncol 37:4012-4012 2019
16 Cheng A-L, et al. Annals of Oncology 30 2019
17 Sicklick et al., Nat Med, April 2019 doi:10.1038/s41591-019-0407-5
18 Ali et al., PLoS Med. 13:e1002194 2016
19 Newman et al., Nat Methods 12:453-457 2015
20 Aran, Hu & Butte, Genome Biol 18:220 2017
21 Binneweis et al., Nat Med 24:541-550 2018
22 Cristescu et al., Science 362 2018
23 Jiang et al. Nat Med. 2018
24 Romano et al., PNAS 112(19):6140-6145 2015
25 Yap T A, et al J Clin Oncol 36:3000 2018
26 Feng et al., JCI Insight 2(14):e93652 2017
27 Kagamu et al., Cancer Immunology Research 8:334-344 2020 DOI: 10.1158/2326-6066.CIR-19-0574
28 AJCC Cancer Staging Manual, Eighth Edition 2018
29 Malik, N. & Daymon, M. Improved double immunoenzyme labelling using alkaline phosphatase and horseradish peroxidase. J. Clin. Pathol. 35, 1092-1094 1982
30 Lan, H. Y., Mu, W., Nikolic-Paterson, D. J. & Atkins, R. C. A novel, simple, reliable, and sensitive method for multiple immunoenzyme staining: use of microwave oven heating to block antibody crossreactivity and retrieve antigens: J. Histochem. Cytochem. 2017 doi:10.1177/43.1.7822770.
31 Ferri, G.-L., Gaudio, R. M., Castello, I. F., Berger, P. & Giro, G. Quadruple Immunofluorescence: A Direct Visualization Method: J. Histochem. Cytochem. 2016 doi:10.1177/002215549704500201
32 Boorsma, D. M. Direct immunoenzyme double staining applicable for monoclonal antibodies. Histochemistry 80, 103-106 1984
33 Zhang, W. et al. Fully automated 5-plex fluorescent immunohistochemistry with tyramide signal amplification and same species antibodies. Lab. Invest. 97, 873-885 2017
34 Shi, S. R., Key, M. E. & Kalra, K. L. Antigen retrieval in formalin-fixed, paraffin-embedded tissues: an enhancement method for immunohistochemical staining based on microwave oven heating of tissue sections. J. Histochem. Cytochem. Off. J. Histochem. Soc. 39, 741-748 1991
35 Shi, S.-R., Shi, Y. & Taylor, C. R. Antigen Retrieval Immunohistochemistry. J. Histochem. Cytochem. 59, 13-32 2011
36 Hennig, C., Adams, N. & Hansen, G. A versatile platform for comprehensive chip-based explorative cytometry. Cytometry A 75A, 362-370 2009
37 Giesen, C. et al. Highly multiplexed imaging of tumor tissues with subcellular resolution by mass cytometry. Nat. Methods 11, 417-422 2014
38 Angelo, M. et al. Multiplexed ion beam imaging (MIBI) of human breast tumors. Nat. Med. 20, 436-442 2014
39 Bandura, D. R. et al. Mass Cytometry: Technique for Real Time Single Cell Multitarget Immunoassay Based on Inductively Coupled Plasma Time-of-Flight Mass Spectrometry. Anal. Chem. 81, 6813-6822 2009
40 Mavropoulos, A. et al. Equivalence of Imaging Mass Cytometry and Immunofluorescence on FFPE Tissue Sections, 2017
41 Wagner, J. et al. A Single-Cell Atlas of the Tumor and Immune Ecosystem of Human Breast Cancer. Cell 177, 1330-1345.e18 2019
42 Zhang, T. et al. Immunocyte Profiling Using Single-Cell Mass Cytometry Reveals EpCAM+CD4+ T Cells Abnormal in Colon Cancer. Front. Immunol. 10 2019
43 Ijsselsteijn, M. E., van der Breggen, R., Farina Sarasqueta, A., Koning, F. & de Miranda, N. F. C. C. A 40-Marker Panel for High Dimensional Characterization of Cancer Immune Microenvironments by Imaging Mass Cytometry. Front. Immunol. 10 2019
44 Han, G., Spitzer, M. H., Bendall, S. C., Fantl, W. J. & Nolan, G. P. Metal-isotope-tagged monoclonal antibodies for high-dimensional mass cytometry. Nat. Protoc. 13, 2121-2148 2018
45 International Classification of Diseases for Oncology, Third Edition (ICD-O-3) WHO 2000
46 Lee et al., Science Immunology 5 eaba0759 2020
47 Forner A, Reig M, Bruix J, Lancet 391:1301-1314 2018
48 Llovet J M et al. N Engl J Med 359:378-90 2008
49 Cheng A L, et al. Lancet Oncol 10:25-34 2009
50 Bruix J, et al. Lancet 389:56-66 2017
51 Kudo M, et al. Lancet 391:1163-1173 2018
52 Abou-Alfa G K, et al. N Engl J Med 379:54-63 2018
53 Zhu A X, et al. Lancet Oncol 20:282-296 2019
54 Dao, Tao et al. Oncoimmunology 8(7) 1570778 2019 doi:10.1080/2162402X.2019.1570778
55 Kabat, E. A. et al., 1991, “Sequences of Proteins of Immunological Interest”, 5th edit., NIH Publication no. 91-3242, U.S. Department of Health and Human Services
56 Chothia, C. & Lesk, A. M., 1987, “Canonical structures for the hypervariable regions of immunoglobulins”, J. Mol. Biol., 196, 901-917
57 Lefranc, M. P., “Unique database numbering system for immunogenetic analysis”, Immunol. Today, 18, 50 1997
58 Lefranc M. P., IMGT unique numbering for immunoglobulin and T cell receptor variable domains and Ig superfamily V-like domains, Dev Comp Immunol. 27(1):55-77 2003
59 Creighton, Proteins, W. H. Freeman and Company (1984)
60 Dordo et al, J. Mol Biol, 1999, 217, 721-739
61 Taylor et al, J. Theor. Biol. 119(1986); 205-218
62 S. French and B. Robson, J. Mol. Evol. 19(1983)171
63 Strohl, BioDrugs (2015) 29:215-239
64 GuI et al., “Antibody-Dependent Phagocytosis of Tumor Cells by Macrophages: A Potent Effector Mechanism of Monoclonal Antibody Therapy of Cancer”, Cancer Res., 75(23), Dec. 1, 2015
65 Lazar et al., 2006, Proc. Natl. Acad. Sci. U.S.A., March 14; 103(11):4005-10
66 Dall et al., Immunol 2002; 169:5171-5180
67 Natsume et al., 2009, Drug Des. Devel. Ther., 3:7-16 or by Zhou Q., Biotechnol. Bioeng., 2008, Feb. 15, 99(3):652-65)
68 Shields et al., 2001, J. Biol. Chem., March 2; 276(9):6591-604)
69 Idusogie et al., J. Immunol., 2001, 166:2571-2575
70 Natsume et al., 2008, Cancer Res., 68: 3863-3872
71 Bruix J, et al. Lancet 389:56-66 2017
72 Kudo M, et al. Lancet 391:1163-1173 2018
73 Abou-Alfa G K, et al. N Engl J Med 379:54-63 2018
74 Zhu A X, et al. Lancet Oncol 20:282-296 2019
75 Kroemer et al. Immunologic Cell Death in Cancer Therapy, Ann Rev Immunol. 31:51-72 2013
76 Galluzzi, Zitvogel & Kroemer Canc. Imm. Res. 4:895-902 2016
77 NCSS Statistical Software, Chapter 546, One ROC Curve and Cutoff Analysis. Documentation for NCSS 2020 Statistical Software.
78 Ito T, et al. Immunity 28(6):870-880 2008

TABLE B
Biomarker summary
One or more of the following biomarkers is assessed within the TME,
in an area of tumour core in a tissue section.

| | | Notes of preferred | | Example measurement |
|---|---|---|---|---|
| Biomarker | Detail | method | Units | (e.g., in HCC) |
| ICOS+ | the density or concentration | counts all ICOS+ cells, | number | >120 cells per mm² |
| density | of cells that express ICOS | including ICOS single | of cells | >100 cells per mm² |
| | protein, i.e., ICOS positive | positive and ICOS | per | in HCC known to be |
| | (ICOS+) cells | FOXP3 double positive | region or | associated with hepatitis |
| | | | area, e.g., | B virus (HBV) infection |
| | | | cells per | OR is stage 2 or later |
| | | | mm² | HCC according to the |
| | | | | criteria of the American |
| | | | | Joint Committee on |
| | | | | Cancer (AJCC2) |
| ICOS+ TReg | the proportion of FOXP3+ | a measure of TReg | fraction | >0.5 |
| proportion | cells which are ICOS+, | activity, indicating | or % | |
| | representing the proportion | the level of immuno- | | |
| | of TReg that are ICOS | suppression in the | | |
| | positive | TME | | |
| Intercellular | ICOS+ FOXP3−: ICOS+ | measured from nucle- | distance, | <105 μm |
| proximity | FOXP3+ intercellular | ar centre to nuclear | e.g., μm | |
| | proximity, being the | centre | | |
| | average (mean) distance | | | |
| | between each ICOS single | | | |
| | positive cell (ICOS+ | | | |
| | FOXP3− cell) and its | | | |
| | nearest ICOS FOXP3 double | | | |
| | positive (ICOS+ FOXP3+) | | | |
| | cell, representing the | | | |
| | proximity of ICOS positive | | | |
| | TReg to other ICOS positive | | | |
| | cells (including ICOS+ | | | |
| | TEff) | | | |
| Zonal | ratio of the number of ICOS | radius measured from | fraction | >0.1 |
| influence | FOXP3 double positive cells | the nuclear centre of | or % | |
| ratio | that are within a defined | the ICOS single posi- | | |
| | proximity (“radius of | tive cell, counting | | |
| | influence”) of one or | all ICOS+ FOXP3+ | | |
| | more ICOS single positive | cells that are within | | |
| | cells to the total number | the region defined by | | |
| | of ICOS single positive | the radius | | |
| | cells. The radius of | | | |
| | influence represents a | | | |
| | distance (e.g., 30 μm) | | | |
| | across which cell-cell | | | |
| | and/or cytokine-dependent | | | |
| | communication can occur | | | |
| | between an ICOS FOXP3 | | | |
| | double positive cell and | | | |
| | an ICOS single positive | | | |
| | cell, this subset of double | | | |
| | positive cells thus | | | |
| | representing TReg that | | | |
| | are positioned to be able | | | |
| | to suppress one or more TEff | | | |

TABLE C
Antibody constant region sequences

| Seq | | | | |
|---|---|---|---|---|
| ID | | | | |
| No: | | | Description | Sequence |
| 15 | Human | IGHG1*01 | Human Heavy Chain Constant | gcctccaccaagggcccatcggtcttccccctggcaccctcctccaagagcacctctg |
| | IgG1 | | Region (IGHG1*01) | ggggcacagcggccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | | tcctcaggactctactccctcagcagcgtggtgaccgtgccctccagcagcttgggca |
| | | | | cccagacctacatctgcaacgtgaatcacaagcccagcaacaccaaggtggacaagaa |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgggtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacgtcttctcatgctccg |
| | | | | tgatgcatgaggctctgcacaaccactacacgcagaagagcctctccctgtctccggg |
| | | | | taaa |
| 16 | | | Human Heavy Chain Constant | ASTKGPSVFPLAPSSKSTSGGTAALGCLVKDYFPEPVTVSWNSGALTSGVHTFPAVLQ |
| | | | Region (IGHG1*01) Protein | SSGLYSLSSVVTVPSSSLGTQTYICNVNHKPSNTKVDKKVEPKSCDKTHTCPPCPAPE |
| | | | Sequence (P01857) | LLGGPSVFLFPPKPKDTLMISRTPEVTCVVVDVSHEDPEVKFNWYVDGVEVHNAKTKP |
| | | | | REEQYNSTYRVVSVLTVLHQDWLNGKEYKCKVSNKALPAPIEKTISKAKGQPREPQVY |
| | | | | TLPPSRDELTKNQVSLTCLVKGFYPSDIAVEWESNGQPENNYKTTPPVLDSDGSFFLY |
| | | | | SKLTVDKSRWQQGNVFSCSVMHEALHNHYTQKSLSLSPGK |
| 17 | Human | IGHG1*02 | Human Heavy Chain Constant | gcctccaccaagggcccatcggtcttccccctggcaccctcctccaagagcacctctg |
| | IgG1 | or | Region (IGHG1*02 or | ggggcacagcggccctgggctgcctggtcaaggactacttccccgaaccggtgacggt |
| | constant | IGHG1*05 | IGHG1*05) Nucleotide | gtcgtggaactcaggcgccctgaccagcggcgtgcacaccttcccggctgtcctacag |
| | region | | Sequence | tcctcaggactctactccctcagcagcgtggtgaccgtgccctccagcagcttgggca |
| | | | | cccagacctacatctgcaacgtgaatcacaagcccagcaacaccaaggtggacaagaa |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacgtcttctcatgctccg |
| | | | | tgatgcatgaggctctgcacaaccactacacgcagaagagcctctccctgtctccggg |
| | | | | taaa |
| 18 | | | Human Heavy Chain Constant | A S T K G P S V F P L A P S S K S T S G G T A A L G C L V K |
| | | | Region (IGHG1*02) Protein | D Y F P E P V T V S W N S G A L T S G V H T F P A V L Q S S |
| | | | Sequence | G L Y S L S S V V T V P S S S L G T Q T Y I C N V N H K P S |
| | | | | N T K V D K K V E P K S C D K T H T C P P C P A P E L L G G |
| | | | | P S V F L F P P K P K D T L M I S R T P E V T C V V V D V S |
| | | | | H E D P E V K F N W Y V D G V E V H N A K T K P R E E Q Y N |
| | | | | S T Y R V V S V L T V L H Q D W L N G K E Y K C K V S N K A |
| | | | | L P A P I E K T I S K A K G Q P R E P Q V Y T L P P S R D E |
| | | | | L T K N Q V S L T C L V K G F Y P S D I A V E W E S N G Q P |
| | | | | E N N Y K T T P P V L D S D G S F F L Y S K L T V D K S R W |
| | | | | Q Q G N V F S C S V M H E A L H N H Y T Q K S L S L S P G K |
| 19 | Human | IGHG1*03 | Human Heavy Chain Constant | gcctccaccaagggcccatcggtcttccccctggcaccctcctccaagagcacctctg |
| | IgG1 | | Region (IGHG1*03) | ggggcacagcggccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | (Y14737) | tcctcaggactctactccctcagcagcgtggtgaccgtgccctccagcagcttgggca |
| | | | | cccagacctacatctgcaacgtgaatcacaagcccagcaacaccaaggtggacaagag |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggaggagatgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctat |
| | | | | agcaagctcaccgtggacaagagcaggtggcagcaggggaacgtcttctcatgctccg |
| | | | | tgatgcatgaggctctgcacaaccactacacgcagaagagcctctccctgtccccggg |
| | | | | taaa |
| 20 | | | Human Heavy Chain Constant | A S T K G P S V F P L A P S S K S T S G G T A A L G C L V K |
| | | | Region (IGHG1*03) Protein | D Y F P E P V T V S W N S G A L T S G V H T F P A V L Q S S |
| | | | Sequence | G L Y S L S S V V T V P S S S L G T Q T Y I C N V N H K P S |
| | | | | N T K V D K R V E P K S C D K T H T C P P C P A P E L L G G |
| | | | | P S V F L F P P K P K D T L M I S R T P E V T C V V V D V S |
| | | | | H E D P E V K F N W Y V D G V E V H N A K T K P R E E Q Y N |
| | | | | S T Y R V V S V L T V L H Q D W L N G K E Y K C K V S N K A |
| | | | | L P A P I E K T I S K A K G Q P R E P Q V Y T L P P S R E E |
| | | | | M T K N Q V S L T C L V K G F Y P S D I A V E W E S N G Q P |
| | | | | E N N Y K T T P P V L D S D G S F F L Y S K L T V D K S R W |
| | | | | Q Q G N V F S C S V M H E A L H N H Y T Q K S L S L S P G K |
| 21 | Human | IGHG1*04 | Human Heavy Chain Constant | gcctccaccaagggcccatcggtcttccccctggcaccctcctccaagagcacctctg |
| | IgG1 | | Region (IGHG1*04) | ggggcacagcggccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | | tcctcaggactctactccctcagcagcgtggtgaccgtgccctccagcagcttgggca |
| | | | | cccagacctacatctgcaacgtgaatcacaagcccagcaacaccaaggtggacaagaa |
| | | | | agttgagcccaaatcttgtgacaaaactcacacatgcccaccgtgcccagcacctgaa |
| | | | | ctcctggggggaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacatcttctcatgctccg |
| | | | | tgatgcatgaggctctgcacaaccactacacgcagaagagcctctccctgtctccggg |
| | | | | taaa |
| 22 | | | Human Heavy Chain Constant | ASTKGPSVFPLAPSSKSTSGGTAALGCLVKDYFPEPVTVSWNSGALTSGVHTFPAVLQ |
| | | | Region (IGHG1*04) Protein | SSGLYSLSSVVTVPSSSLGTQTYICNVNHKPSNTKVDKKVEPKSCDKTHTCPPCPAPE |
| | | | Sequence | LLGGPSVFLFPPKPKDTLMISRTPEVTCVVVDVSHEDPEVKFNWYVDGVEVHNAKTKP |
| | | | | REEQYNSTYRVVSVLTVLHQDWLNGKEYKCKVSNKALPAPIEKTISKAKGQPREPQVY |
| | | | | TLPPSRDELTKNQVSLTCLVKGFYPSDIAVEWESNGQPENNYKTTPPVLDSDGSFFLY |
| | | | | SKLTVDKSRWQQGNIFSCSVMHEALHNHYTQKSLSLSPGK |
| 23 | Disabled | Disabled | Disabled Human IGHG1*01 | gcctccaccaagggcccatcggtcttccccctggcaccctcctccaagagcacctctg |
| | Human | human | Heavy Chain Constant | ggggcacagcggccctgggctgcctggtcaaggactacttccccgaaccggtgacggt |
| | IgG1 | IGHG1*01 | Region Nucleotide Sequence | gtcgtggaactcaggcgccctgaccagcggcgtgcacaccttcccggctgtcctacag |
| | heavy | | | tcctcaggactctactccctcagcagcgtggtgaccgtgccctccagcagcttgggca |
| | chain | | | cccagacctacatctgcaacgtgaatcacaagcccagcaacaccaaggtggacaagaa |
| | constant | | | agtggagcccaaatcttgtgacaaaactcacacatgcccaccgtgcccagcacctgaa |
| | region | | | ctcgcgggggcaccgtcagtcttcctcttccccccaaaacccaaggacaccctcatga |
| | | | | tctcccggacccctgaggtcacatgcgtggtggtggacgtgagccacgaagaccctga |
| | | | | ggtcaagttcaactggtacgtggacggcgtggaggtgcataatgccaagacaaagccg |
| | | | | cgggaggagcagtacaacagcacgtaccgtgtggtcagcgtcctcaccgtcctgcacc |
| | | | | aggactggctgaatggcaaggagtacaagtgcaaggtctccaacaaagccctcccagc |
| | | | | ccccatcgagaaaaccatctccaaagccaaagggcagccccgagaaccacaggtgtac |
| | | | | accctgcccccatcccgggatgagctgaccaagaaccaggtcagcctgacctgcctgg |
| | | | | tcaaaggcttctatcccagcgacatcgccgtggagtgggagagcaatgggcagccgga |
| | | | | gaacaactacaagaccacgcctcccgtgctggactccgacggctccttcttcctctac |
| | | | | agcaagctcaccgtggacaagagcaggtggcagcaggggaacgtcttctcatgctccg |
| | | | | tgatgcatgaggctctgcacaaccactacacgcagaagagcctctccctgtctccggg |
| | | | | taaa |
| 24 | | | Disabled Human IGHG1*01 | ASTKGPSVFPLAPSSKSTSGGTAALGCLVKDYFPEPVTVSWNSGALTSGVHTFPAVLQ |
| | | | Heavy Chain Constant | SSGLYSLSSVVTVPSSSLGTQTYICNVNHKPSNTKVDKKVEPKSCDKTHTCPPCPAPE |
| | | | Region Amino Acid | LAGAPSVFLFPPKPKDTLMISRTPEVTCVVVDVSHEDPEVKFNWYVDGVEVHNAKTKP |
| | | | Sequence. Two residues | REEQYNSTYRVVSVLTVLHQDWLNGKEYKCKVSNKALPAPIEKTISKAKGQPREPQVY |
| | | | that differ from the wild- | TLPPSRDELTKNQVSLTCLVKGFYPSDIAVEWESNGQPENNYKTTPPVLDSDGSFFLY |
| | | | type sequence are | SKLTVDKSRWQQGNVFSCSVMHEALHNHYTQKSLSLSPGK |
| | | | identified in bold | |
| 25 | Human | IGHG2*01 | Human Heavy Chain Constant | gcctccaccaagggcccatcggtcttccccctggcgccctgctccaggagcacctccg |
| | IgG2 | or | Region (IGHG2*01 or | agagcacagccgccctgggctgcctggtcaaggactacttccccgaaccggtgacggt |
| | constant | IGHG2*04 | IGHG2*03 or IGHG2*05) | gtcgtggaactcaggcgctctgaccagcggcgtgcacaccttcccagctgtcctacag |
| | region | or | Nucleotide Sequence | tcctcaggactctactccctcagcagcgtggtgaccgtgccctccagcaacttcggca |
| | | IGHG2*05 | | cccagacctacacctgcaacgtagatcacaagcccagcaacaccaaggtggacaagac |
| | | | | agttgagcgcaaatgttgtgtcgagtgcccaccgtgcccagcaccacctgtggcagga |
| | | | | ccgtcagtcttcctcttccccccaaaacccaaggacaccctcatgatctcccggaccc |
| | | | | ctgaggtcacgtgcgtggtggtggacgtgagccacgaagaccccgaggtccagttcaa |
| | | | | ctggtacgtggacggcgtggaggtgcataatgccaagacaaagccacgggaggagcag |
| | | | | ttcaacagcacgttccgtgtggtcagcgtcctcaccgttgtgcaccaggactggctga |
| | | | | acggcaaggagtacaagtgcaaggtctccaacaaaggcctcccagcccccatcgagaa |
| | | | | aaccatctccaaaaccaaagggcagccccgagaaccacaggtgtacaccctgccccca |
| | | | | tcccgggaggagatgaccaagaaccaggtcagcctgacctgcctggtcaaaggcttct |
| | | | | accccagcgacatcgccgtggagtgggagagcaatgggcagccggagaacaactacaa |
| | | | | gaccacacctcccatgctggactccgacggctccttcttcctctacagcaagctcacc |
| | | | | gtggacaagagcaggtggcagcaggggaacgtcttctcatgctccgtgatgcatgagg |
| | | | | ctctgcacaaccactacacgcagaagagcctctccctgtctccgggtaaa |
| 26 | | | Human Heavy Chain Constant | ASTKGPSVFPLAPCSRSTSESTAALGCLVKDYFPEPVTVSWNSGALTSGVHTFPAVLQ |
| | | | Region (IGHG2*01) Protein | SSGLYSLSSVVTVPSSNFGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGVEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDIAVEWESNGQPENNYKTTPPMLDSDGSFFLYSKLT |
| | | | | VDKSRWQQGNVFSCSVMHEALHNHYTQKSLSLSPGK |
| 27 | Human | IGHG2*02 | Human Heavy Chain Constant | GCCTCCACCAAGGGCCCATCGGTCTTCCCCCTGGCGCCCTGCTCCAGGAGCACCTCCG |
| | IgG2 | | Region (IGHG2*02) | AGAGCACAGCGGCCCTGGGCTGCCTGGTCAAGGACTACTTCCCCGAACCGGTGACGGT |
| | constant | | Nucleotide Sequence | GTCGTGGAACTCAGGCGCTCTGACCAGCGGCGTGCACACCTTCCCGGCTGTCCTACAG |
| | region | | | TCCTCAGGACTCTACTCCCTCAGCAGCGTGGTGACCGTGACCTCCAGCAACTTCGGCA |
| | | | | CCCAGACCTACACCTGCAACGTAGATCACAAGCCCAGCAACACCAAGGTGGACAAGAC |
| | | | | AGTTGAGCGCAAATGTTGTGTCGAGTGCCCACCGTGCCCAGCACCACCTGTGGCAGGA |
| | | | | CCGTCAGTCTTCCTCTTCCCCCCAAAACCCAAGGACACCCTCATGATCTCCCGGACCC |
| | | | | CTGAGGTCACGTGCGTGGTGGTGGACGTGAGCCACGAAGACCCCGAGGTCCAGTTCAA |
| | | | | CTGGTACGTGGACGGCATGGAGGTGCATAATGCCAAGACAAAGCCACGGGAGGAGCAG |
| | | | | TTCAACAGCACGTTCCGTGTGGTCAGCGTCCTCACCGTCGTGCACCAGGACTGGCTGA |
| | | | | ACGGCAAGGAGTACAAGTGCAAGGTCTCCAACAAAGGCCTCCCAGCCCCCATCGAGAA |
| | | | | AACCATCTCCAAAACCAAAGGGCAGCCCCGAGAACCACAGGTGTACACCCTGCCCCCA |
| | | | | TCCCGGGAGGAGATGACCAAGAACCAGGTCAGCCTGACCTGCCTGGTCAAAGGCTTCT |
| | | | | ACCCCAGCGACATCGCCGTGGAGTGGGAGAGCAATGGGCAGCCGGAGAACAACTACAA |
| | | | | GACCACACCTCCCATGCTGGACTCCGACGGCTCCTTCTTCCTCTACAGCAAGCTCACC |
| | | | | GTGGACAAGAGCAGGTGGCAGCAGGGGAACGTCTTCTCATGCTCCGTGATGCATGAGG |
| | | | | CTCTGCACAACCACTACACACAGAAGAGCCTCTCCCTGTCTCCGGGTAAA |
| 28 | | | Human Heavy Chain Constant | ASTKGPSVFPLAPCSRSTSESTAALGCLVKDYFPEPVTVSWNSGALTSGVHTFPAVLQ |
| | | | Region (IGHG2*02) Protein | SSGLYSLSSVVTVTSSNFGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGMEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDIAVEWESNGQPENNYKTTPPMLDSDGSFFLYSKLT |
| | | | | VDKSRWQQGNVFSCSVMHEALHNHYTQKSLSLSPGK |
| 29 | Human | IGHG2*04 | Human Heavy Chain Constant | gcctccaccaagggcccatcggtcttccccctggcgccctgctccaggagcacctccg |
| | IgG2 | | Region (IGHG2*04) | agagcacagcggccctgggctgcctggtcaaggactacttccccgaaccggtgacggt |
| | constant | | Nucleotide Sequence | gtcgtggaactcaggcgctctgaccagcggcgtgcacaccttcccagctgtcctacag |
| | region | | | tcctcaggactctactccctcagcagcgtggtgaccgtgccctccagcagcttgggca |
| | | | | cccagacctacacctgcaacgtagatcacaagcccagcaacaccaaggtggacaagac |
| | | | | agttgagcgcaaatgttgtgtcgagtgcccaccgtgcccagcaccacctgtggcagga |
| | | | | ccgtcagtcttcctcttccccccaaaacccaaggacaccctcatgatctcccggaccc |
| | | | | ctgaggtcacgtgcgtggtggtggacgtgagccacgaagaccccgaggtccagttcaa |
| | | | | ctggtacgtggacggcgtggaggtgcataatgccaagacaaagccacgggaggagcag |
| | | | | ttcaacagcacgttccgtgtggtcagcgtcctcaccgttgtgcaccaggactggctga |
| | | | | acggcaaggagtacaagtgcaaggtctccaacaaaggcctcccagcccccatcgagaa |
| | | | | aaccatctccaaaaccaaagggcagccccgagaaccacaggtgtacaccctgccccca |
| | | | | tcccgggaggagatgaccaagaaccaggtcagcctgacctgcctggtcaaaggcttct |
| | | | | accccagcgacatcgccgtggagtgggagagcaatgggcagccggagaacaactacaa |
| | | | | gaccacacctcccatgctggactccgacggctccttcttcctctacagcaagctcacc |
| | | | | gtggacaagagcaggtggcagcaggggaacgtcttctcatgctccgtgatgcatgagg |
| | | | | ctctgcacaaccactacacgcagaagagcctctccctgtctccgggtaaa |
| 30 | | | Human Heavy Chain Constant | ASTKGPSVFPLAPCSRSTSESTAALGCLVKDYFPEPVTVSWNSGALTSGVHTFPAVLQ |
| | | | Region (IGHG2*04) Protein | SSGLYSLSSVVTVPSSSLGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGVEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDIAVEWESNGQPENNYKTTPPMLDSDGSFFLYSKLT |
| | | | | VDKSRWQQGNVFSCSVMHEALHNHYTQKSLSLSPGK |
| 31 | Human | IGHG2*06 | Human Heavy Chain Constant | GCCTCCACCAAGGGCCCATCGGTCTTCCCCCTGGCGCCCTGCTCCAGGAGCACCTCCG |
| | IgG2 | | Region (IGHG2*06) | AGAGCACAGCGGCCCTGGGCTGCCTGGTCAAGGACTACTTCCCCGAACCGGTGACGGT |
| | constant | | Nucleotide Sequence | GTCGTGGAACTCAGGCGCTCTGACCAGCGGCGTGCACACCTTCCCGGCTGTCCTACAG |
| | region | | | TCCTCAGGACTCTACTCCCTCAGCAGCGTGGTGACCGTGCCCTCCAGCAACTTCGGCA |
| | | | | CCCAGACCTACACCTGCAACGTAGATCACAAGCCCAGCAACACCAAGGTGGACAAGAC |
| | | | | AGTTGAGCGCAAATGTTGTGTCGAGTGCCCACCGTGCCCAGCACCACCTGTGGCAGGA |
| | | | | CCGTCAGTCTTCCTCTTCCCCCCAAAACCCAAGGACACCCTCATGATCTCCCGGACCC |
| | | | | CTGAGGTCACGTGCGTGGTGGTGGACGTGAGCCACGAAGACCCCGAGGTCCAGTTCAA |
| | | | | CTGGTACGTGGACGGCGTGGAGGTGCATAATGCCAAGACAAAGCCACGGGAGGAGCAG |
| | | | | TTCAACAGCACGTTCCGTGTGGTCAGCGTCCTCACCGTCGTGCACCAGGACTGGCTGA |
| | | | | ACGGCAAGGAGTACAAGTGCAAGGTCTCCAACAAAGGCCTCCCAGCCCCCATCGAGAA |
| | | | | AACCATCTCCAAAACCAAAGGGCAGCCCCGAGAACCACAGGTGTACACCCTGCCCCCA |
| | | | | TCCCGGGAGGAGATGACCAAGAACCAGGTCAGCCTGACCTGCCTGGTCAAAGGCTTCT |
| | | | | ACCCCAGCGACATCTCCGTGGAGTGGGAGAGCAATGGGCAGCCGGAGAACAACTACAA |
| | | | | GACCACACCTCCCATGCTGGACTCCGACGGCTCCTTCTTCCTCTACAGCAAGCTCACC |
| | | | | GTGGACAAGAGCAGGTGGCAGCAGGGGAACGTCTTCTCATGCTCCGTGATGCATGAGG |
| | | | | CTCTGCACAACCACTACACACAGAAGAGCCTCTCCCTGTCTCCGGGTAAA |
| 32 | | | Human Heavy Chain Constant | ASTKGPSVFPLAPCSRSTSESTAALGCLVKDYFPEPVTVSWNSGALTSGVHTFPAVLQ |
| | | | Region (IGHG2*06) Protein | SSGLYSLSSVVTVPSSNFGTQTYTCNVDHKPSNTKVDKTVERKCCVECPPCPAPPVAG |
| | | | Sequence | PSVFLFPPKPKDTLMISRTPEVTCVVVDVSHEDPEVQFNWYVDGVEVHNAKTKPREEQ |
| | | | | FNSTFRVVSVLTVVHQDWLNGKEYKCKVSNKGLPAPIEKTISKTKGQPREPQVYTLPP |
| | | | | SREEMTKNQVSLTCLVKGFYPSDISVEWESNGQPENNYKTTPPMLDSDGSFFLYSKLT |
| | | | | VDKSRWQQGNVFSCSVMHEALHNHYTQKSLSLSPGK |
| 33 | Human | IGHG4*01 | Human Heavy Chain Constant | gcttccaccaagggcccatccgtcttccccctggcgccctgctccaggagcacctccg |
| | IgG4 | or | Region (IGHG4*01 or | agagcacagccgccctgggctgcctggtcaaggactacttccccgaaccggtgacggt |
| | constant | IGHG4*04 | IGHG4*04) Nucleotide | gtcgtggaactcaggcgccctgaccagcggcgtgcacaccttcccggctgtcctacag |
| | region | | Sequence | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccatgcccatcatgcccagcacctgagttcctgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcctccatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggctccttcttcctctacagcaggcta |
| | | | | accgtggacaagagcaggtggcaggaggggaatgtcttctcatgctccgtgatgcatg |
| | | | | aggctctgcacaaccactacacacagaagagcctctccctgtctctgggtaaa |
| 34 | | | Human Heavy Chain Constant | ASTKGPSVFPLAPCSRSTSESTAALGCLVKDYFPEPVTVSWNSGALTSGVHTFPAVLQ |
| | | | Region (IGHG4*01) Protein | SSGLYSLSSVVTVPSSSLGTKTYTCNVDHKPSNTKVDKRVESKYGPPCPSCPAPEFLG |
| | | | Sequence (P01861) | GPSVFLFPPKPKDTLMISRTPEVTCVVVDVSQEDPEVQFNWYVDGVEVHNAKTKPREE |
| | | | | QFNSTYRVVSVLTVLHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSRL |
| | | | | TVDKSRWQEGNVFSCSVMHEALHNHYTQKSLSLSLGK |
| 35 | Human | IGHG4*02 | Human Heavy Chain Constant | gcttccaccaagggcccatccgtcttccccctggcgccctgctccaggagcacctccg |
| | IgG4 | | Region (IGHG4*02) | agagcacagccgccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccgtgcccatcatgcccagcacctgagttcctgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcgtgcaccaggactggc |
| | | | | tgaacggcaaggagtacaagtgcaaggtctccaacaaaggcctcccgtcctccatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggctccttcttcctctacagcaggcta |
| | | | | accgtggacaagagcaggtggcaggaggggaatgtcttctcatgctccgtgatgcatg |
| | | | | aggctctgcacaaccactacacgcagaagagcctctccctgtctctgggtaaa |
| 36 | | | Human Heavy Chain Constant | ASTKGPSVFPLAPCSRSTSESTAALGCLVKDYFPEPVTVSWNSGALTSGVHTFPAVLQ |
| | | | Region (IGHG4*02) Protein | SSGLYSLSSVVTVPSSSLGTKTYTCNVDHKPSNTKVDKRVESKYGPPCPSCPAPEFLG |
| | | | Sequence | GPSVFLFPPKPKDTLMISRTPEVTCVVVDVSQEDPEVQFNWYVDGVEVHNAKTKPREE |
| | | | | QFNSTYRVVSVLTVVHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSRL |
| | | | | TVDKSRWQEGNVFSCSVMHEALHNHYTQKSLSLSLGK |
| 37 | Human | IGHG4*03 | Human Heavy Chain Constant | gcttccaccaagggcccatccgtcttccccctggcgccctgctccaggagcacctccg |
| | IgG4 | | Region (IGHG4*03) | agagcacagccgccctgggctgcctggtcaaggactacttccccgaaccggtgacggt |
| | constant | | Nucleotide Sequence | gtcgtggaactcaggcgccctgaccagcggcgtgcacaccttcccggctgtcctacag |
| | region | | | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccatgcccatcatgcccagcacctgagttcctgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcctccatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggctccttcttcctctacagcaagctc |
| | | | | accgtggacaagagcaggtggcaggaggggaacgtcttctcatgctccgtgatgcatg |
| | | | | aggctctgcacaaccactacacgcagaagagcctctccctgtctctgggtaaa |
| 38 | | | Human Heavy Chain Constant | ASTKGPSVFPLAPCSRSTSESTAALGCLVKDYFPEPVTVSWNSGALTSGVHTFPAVLQ |
| | | | Region (IGHG4*03) Protein | SSGLYSLSSVVTVPSSSLGTKTYTCNVDHKPSNTKVDKRVESKYGPPCPSCPAPEFLG |
| | | | Sequence | GPSVFLFPPKPKDTLMISRTPEVTCVVVDVSQEDPEVQFNWYVDGVEVHNAKTKPREE |
| | | | | QFNSTYRVVSVLTVLHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSKL |
| | | | | TVDKSRWQEGNVFSCSVMHEALHNHYTQKSLSLSLGK |
| 39 | Human | IGHG4-PE | Human Heavy Chain Constant | gcctccaccaagggcccatccgtcttccccctggcgccctgctccaggagcacctccg |
| | IgG4-PE | | Region (IGHG4-PE) | agagcacggccgccctgggctgcctggtcaaggactacttccccgaaccagtgacggt |
| | constant | | Nucleotide Sequence | gtcgtggaactcaggcgccctgaccagcggcgtgcacaccttcccggctgtcctacag |
| | region | | Version A | tcctcaggactctactccctcagcagcgtggtgaccgtgccctccagcagcttgggca |
| | | | | cgaagacctacacctgcaacgtagatcacaagcccagcaacaccaaggtggacaagag |
| | | | | agttgagtccaaatatggtcccccatgcccaccatgcccagcgcctgaatttgagggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcatcgatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggatccttcttcctctacagcaggcta |
| | | | | accgtggacaagagcaggtggcaggaggggaatgtcttctcatgctccgtgatgcatg |
| | | | | aggctctgcacaaccactacacacagaagagcctctccctgtctctgggtaaa |
| 40 | | | Human Heavy Chain Constant | gcctccaccaagggacctagcgtgttccctctcgccccctgttccaggtccacaagcg |
| | | | Region (IGHG4-PE) | agtccaccgctgccctcggctgtctggtgaaagactactttcccgagcccgtgaccgt |
| | | | Nucleotide Sequence | ctcctggaatagcggagccctgacctccggcgtgcacacatttcccgccgtgctgcag |
| | | | Version B | agcagcggactgtatagcctgagcagcgtggtgaccgtgcccagctccagcctcggca |
| | | | | ccaaaacctacacctgcaacgtggaccacaagccctccaacaccaaggtggacaagcg |
| | | | | ggtggagagcaagtacggccccccttgccctccttgtcctgcccctgagttcgaggga |
| | | | | ggaccctccgtgttcctgtttccccccaaacccaaggacaccctgatgatctcccgga |
| | | | | cacccgaggtgacctgtgtggtcgtggacgtcagccaggaggaccccgaggtgcagtt |
| | | | | caactggtatgtggacggcgtggaggtgcacaatgccaaaaccaagcccagggaggag |
| | | | | cagttcaattccacctacagggtggtgagcgtgctgaccgtcctgcatcaggattggc |
| | | | | tgaacggcaaggagtacaagtgcaaggtgtccaacaagggactgcccagctccatcga |
| | | | | gaagaccatcagcaaggctaagggccagccgagggagccccaggtgtataccctgcct |
| | | | | cctagccaggaagagatgaccaagaaccaagtgtccctgacctgcctggtgaagggat |
| | | | | tctacccctccgacatcgccgtggagtgggagagcaatggccagcccgagaacaacta |
| | | | | caaaacaacccctcccgtgctcgatagcgacggcagcttctttctctacagccggctg |
| | | | | acagtggacaagagcaggtggcaggagggcaacgtgttctcctgttccgtgatgcacg |
| | | | | aggccctgcacaatcactacacccagaagagcctctccctgtccctgggcaag |
| 41 | | | Human Heavy Chain Constant | gccagcaccaagggcccttccgtgttccccctggccccttgcagcaggagcacctccg |
| | | | Region (IGHG4-PE) | aatccacagctgccctgggctgtctggtgaaggactactttcccgagcccgtgaccgt |
| | | | Nucleotide Sequence | gagctggaacagcggcgctctgacatccggcgtccacacctttcctgccgtcctgcag |
| | | | Version C | tcctccggcctctactccctgtcctccgtggtgaccgtgcctagctcctccctcggca |
| | | | | ccaagacctacacctgtaacgtggaccacaaaccctccaacaccaaggtggacaaacg |
| | | | | ggtcgagagcaagtacggccctccctgccctccttgtcctgcccccgagttcgaaggc |
| | | | | ggacccagcgtgttcctgttccctcctaagcccaaggacaccctcatgatcagccgga |
| | | | | cacccgaggtgacctgcgtggtggtggatgtgagccaggaggaccctgaggtccagtt |
| | | | | caactggtatgtggatggcgtggaggtgcacaacgccaagacaaagccccgggaagag |
| | | | | cagttcaactccacctacagggtggtcagcgtgctgaccgtgctgcatcaggactggc |
| | | | | tgaacggcaaggagtacaagtgcaaggtcagcaataagggactgcccagcagcatcga |
| | | | | gaagaccatctccaaggctaaaggccagccccgggaacctcaggtgtacaccctgcct |
| | | | | cccagccaggaggagatgaccaagaaccaggtgagcctgacctgcctggtgaagggat |
| | | | | tctacccttccgacatcgccgtggagtgggagtccaacggccagcccgagaacaatta |
| | | | | taagaccacccctcccgtcctcgacagcgacggatccttctttctgtactccaggctg |
| | | | | accgtggataagtccaggtggcaggaaggcaacgtgttcagctgctccgtgatgcacg |
| | | | | aggccctgcacaatcactacacccagaagtccctgagcctgtccctgggaaag |
| 42 | | | Human Heavy Chain Constant | ASTKGPSVFPLAPCSRSTSESTAALGCLVKDYFPEPVTVSWNSGALTSGVHTFPAVLQ |
| | | | Region (IGHG4-PE) Protein | SSGLYSLSSVVTVPSSSLGTKTYTCNVDHKPSNTKVDKRVESKYGPPCPPCPAPEFEG |
| | | | Sequence | GPSVFLFPPKPKDTLMISRTPEVTCVVVDVSQEDPEVQFNWYVDGVEVHNAKTKPREE |
| | | | | QFNSTYRVVSVLTVLHQDWLNGKEYKCKVSNKGLPSSIEKTISKAKGQPREPQVYTLP |
| | | | | PSQEEMTKNQVSLTCLVKGFYPSDIAVEWESNGQPENNYKTTPPVLDSDGSFFLYSRL |
| | | | | TVDKSRWQEGNVFSCSVMHEALHNHYTQKSLSLSLGK |
| 43 | In- | In- | Inactivated Human Heavy | GCCTCCACCAAGGGCCCATCCGTCTTCCCCCTGGCGCCCTGCTCCAGGAGCACCTCCG |
| | activated | activated | Chain Constant Region | agagcacggccgccctgggctgcctggtcaaggactacttccccgaaccagtgacggt |
| | Human | IGHG4 | (IGHG4) Nucleotide | gtcgtggaactcaggcgccctgaccagcggcgtgcacaccttcccggctgtcctacag |
| | IgG4 | | Sequence | tcctcaggactctactccctcagcagcgtggtgaccgtgccctccagcagcttgggca |
| | constant | | | cgaagacctacacctgcaacgtagatcacaagcccagcaacaccaaggtggacaagag |
| | region | | | agttgagtccaaatatggtcccccatgcccaccatgcccagcgcctccagttgcgggg |
| | | | | ggaccatcagtcttcctgttccccccaaaacccaaggacactctcatgatctcccgga |
| | | | | cccctgaggtcacgtgcgtggtggtggacgtgagccaggaagaccccgaggtccagtt |
| | | | | caactggtacgtggatggcgtggaggtgcataatgccaagacaaagccgcgggaggag |
| | | | | cagttcaacagcacgtaccgtgtggtcagcgtcctcaccgtcctgcaccaggactggc |
| | | | | tgaacggcaaggagtacaagtgcaaggtctccaacaaaggcctcccgtcatcgatcga |
| | | | | gaaaaccatctccaaagccaaagggcagccccgagagccacaggtgtacaccctgccc |
| | | | | ccatcccaggaggagatgaccaagaaccaggtcagcctgacctgcctggtcaaaggct |
| | | | | tctaccccagcgacatcgccgtggagtgggagagcaatgggcagccggagaacaacta |
| | | | | caagaccacgcctcccgtgctggactccgacggatccttcttcctctacagcaggcta |
| | | | | accgtggacaagagcaggtggcaggaggggaatgtcttctcatgctccgtgatgcatg |
| | | | | aggctctgcacaaccactacacacagaagagcctctccctgtctctgggtaaa |
| 44 | | | Inactivated Human Heavy | ASTKGPSVFPLAPCSRSTSESTAALGCLVKDYFPEPVTVSWNSGALTSGVHTFPAVLQ |
| | | | Chain Constant Region | SSGLYSLSSVVTVPSSSLGTKTYTCNVDHKPSNTKVDKRVESKYGPPCPPCPAPPVAG |
| | | | (IGHG4) Protein Sequence | GPSVFLFPPKPKDTLMISRTPEVTCVVVDVSQEDPEVQFNWYVDGVEVHNAKTKPREE |
| | | | (inactivating mutations | QFNSTYRVVSVLTVLHQDWLNGKEYKCKVSNKGLPSSIEKTISKAKGQPREPQVYTLP |
| | | | from human IgG4 shown in | PSQEEMTKNQVSLTCLVKGFYPSDIAVEWESNGQPENNYKTTPPVLDSDGSFFLYSRL |
| | | | bold) | TVDKSRWQEGNVFSCSVMHEALHNHYTQKSLSLSLGK |
| 45 | Human Cκ | IGKC*01 | Human Cκ Light Chain | cgtacggtggccgctccctccgtgttcatcttcccaccttccgacgagcagctgaagt |
| | constant | | Constant Region (IGKC*01) | ccggcaccgcttctgtcgtgtgcctgctgaacaacttctacccccgcgaggccaaggt |
| | region | | Nucleotide Sequence | gcagtggaaggtggacaacgccctgcagtccggcaactcccaggaatccgtgaccgag |
| | | | | caggactccaaggacagcacctactccctgtcctccaccctgaccctgtccaaggccg |
| | | | | actacgagaagcacaaggtgtacgcctgcgaagtgacccaccagggcctgtctagccc |
| | | | | cgtgaccaagtctttcaaccggggcgagtgt |
| 46 | | | Cκ Light Chain Constant | RTVAAPSVFIFPPSDEQLKSGTASVVCLLNNFYPREAKVQWKVDNALQSGNSQESVTE |
| | | | Region (IGKC*01) Amino | QDSKDSTYSLSSTLTLSKADYEKHKVYACEVTHQGLSSPVTKSFNRGEC |
| | | | Acid Sequence | |
| 47 | Human Cκ | IGKC*02 | Cκ Light Chain Constant | cgaactgtggctgcaccatctgtcttcatcttcccgccatctgatgagcagttgaaat |
| | constant | | Region (IGKC*02) | ctggaactgcctctgttgtgtgcctgctgaataacttctatcccagagaggccaaagt |
| | region | | Nucleotide Sequence | acagtggaaggtggataacgccctccaatcgggtaactcccaggagagtgtcacagag |
| | | | | caggagagcaaggacagcacctacagcctcagcagcaccctgacgctgagcaaagcag |
| | | | | actacgagaaacacaaagtctacgccggcgaagtcacccatcagggcctgagctcgcc |
| | | | | cgtcacaaagagcttcaacaggggagagtgt |
| 48 | | | Cκ Light Chain Constant | RTVAAPSVFIFPPSDEQLKSGTASVVCLLNNFYPREAKVQWKVDNALQSGNSQESVTE |
| | | | Region (IGKC*02) Amino | QESKDSTYSLSSTLTLSKADYEKHKVYAGEVTHQGLSSPVTKSFNRGEC |
| | | | Acid Sequence | |
| 49 | Human Cκ | TGKC*03 | Cκ Light Chain Constant | cgaactgtggctgcaccatctgtcttcatcttcccgccatctgatgagcagttgaaat |
| | constant | | Region (IGKC*03) | ctggaactgcctctgttgtgtgcctgctgaataacttctatcccagagaggccaaagt |
| | region | | Nucleotide Sequence | acagcggaaggtggataacgccctccaatcgggtaactcccaggagagtgtcacagag |
| | | | | caggagagcaaggacagcacctacagcctcagcagcaccctgacgctgagcaaagcag |
| | | | | actacgagaaacacaaagtctacgcctgcgaagtcacccatcagggcctgagctcgcc |
| | | | | cgtcacaaagagcttcaacaggggagagtgt |
| 50 | | | Cκ Light Chain Constant | RTVAAPSVFIFPPSDEQLKSGTASVVCLLNNFYPREAKVQRKVDNALQSGNSQESVTE |
| | | | Region (IGKC*03) Amino | QESKDSTYSLSSTLTLSKADYEKHKVYACEVTHQGLSSPVTKSFNRGEC |
| | | | Acid Sequence | |
| 51 | Human Cκ | IGKC*04 | Cκ Light Chain Constant | cgaactgtggctgcaccatctgtcttcatcttcccgccatctgatgagcagttgaaat |
| | constant | | Region (IGKC*04) | ctggaactgcctctgttgtgtgcctgctgaataacttctatcccagagaggccaaagt |
| | region | | Nucleotide Sequence | acagtggaaggtggataacgccctccaatcgggtaactcccaggagagtgtcacagag |
| | | | | caggacagcaaggacagcacctacagcctcagcagcaccctgacgctgagcaaagcag |
| | | | | actacgagaaacacaaactctacgcctgcgaagtcacccatcagggcctgagctcgcc |
| | | | | cgtcacaaagagcttcaacaggggagagtgt |
| 52 | | | Cκ Light Chain Constant | RTVAAPSVFIFPPSDEQLKSGTASVVCLLNNFYPREAKVQWKVDNALQSGNSQESVTE |
| | | | Region (IGKC*04) Amino | QDSKDSTYSLSSTLTLSKADYEKHKLYACEVTHQGLSSPVTKSFNRGEC |
| | | | Acid Sequence | |
| 53 | Human Cκ | IGKC*05 | Cκ Light Chain Constant | cgaactgtggctgcaccatctgtcttcatcttcccgccatctgatgagcagttgaaat |
| | constant | | Region (IGKC*05) | ctggaactgcctctgttgtgtgcctgctgaataacttctatcccagagaggccaaagt |
| | region | | Nucleotide Sequence | acagtggaaggtggataacgccctccaatcgggtaactcccaggagagtgtcacagag |
| | | | | caggacagcaaggacagcacctacagcctcagcaacaccctgacgctgagcaaagcag |
| | | | | actacgagaaacacaaagtctacgcctgcgaagtcacccatcagggcctgagctcgcc |
| | | | | cgtcacaaagagcttcaacaggggagagtgc |
| 54 | | | Ck Light Chain Constant | RTVAAPSVFIFPPSDEQLKSGTASVVCLLNNFYPREAKVQWKVDNALQSGNSQESVTE |
| | | | Region (IGKC*05) Amino | QDSKDSTYSLSNTLTLSKADYEKHKVYACEVTHQGLSSPVTKSFNRGEC |
| | | | Acid Sequence | |
| 55 | Human Cλ | IGLC1*01 | Cλ Light Chain Constant | cccaaggccaaccccacggtcactctgttcccgccctcctctgaggagctccaagcca |
| | constant | | Region (IGLC1*01) | acaaggccacactagtgtgtctgatcagtgacttctacccgggagctgtgacagtggc |
| | region | | Nucleotide Sequence | ttggaaggcagatggcagccccgtcaaggcgggagtggagacgaccaaaccctccaaa |
| | | | (ENST00000390321.2) | cagagcaacaacaagtacgcggccagcagctacctgagcctgacgcccgagcagtgga |
| | | | | agtcccacagaagctacagctgccaggtcacgcatgaagggagcaccgtggagaagac |
| | | | | agtggcccctacagaatgttca |
| 56 | | | Cλ Light Chain Constant | PKANPTVTLFPPSSEELQANKATLVCLISDFYPGAVTVAWKADGSPVKAGVETTKPSK |
| | | | Region (IGLC1*01) Amino | QSNNKYAASSYLSLTPEQWKSHRSYSCQVTHEGSTVEKTVAPTECS |
| | | | Acid Sequence (A0A075B6K8) | |
| 57 | Human Cλ | IGLC1*02 | Cλ Light Chain Constant | ggtcagcccaaggccaaccccactgtcactctgttcccgccctcctctgaggagctcc |
| | constant | | Region (IGLC1*02) | aagccaacaaggccacactagtgtgtctgatcagtgacttctacccgggagctgtgac |
| | region | | Nucleotide Sequence | agtggcctggaaggcagatggcagccccgtcaaggcgggagtggagaccaccaaaccc |
| | | | Version A | tccaaacagagcaacaacaagtacgcggccagcagctacctgagcctgacgcccgagc |
| | | | | agtggaagtcccacagaagctacagctgccaggtcacgcatgaagggagcaccgtgga |
| | | | | gaagacagtggcccctacagaatgttca |
| 58 | | | Cλ Light Chain Constant | ggtcagcccaaggccaaccccactgtcactctgttcccgccctcctctgaggagctcc |
| | | | Region (IGLC1*02) | aagccaacaaggccacactagtgtgtctgatcagtgacttctacccgggagctgtgac |
| | | | Nucleotide Sequence | agtggcctggaaggcagatggcagccccgtcaaggcgggagtggagaccaccaaaccc |
| | | | Version B | tccaaacagagcaacaacaagtacgcggccagcagctacctgagcctgacgcccgagc |
| | | | | agtggaagtcccacagaagctacagctgccaggtcacgcatgaagggagcaccgtgga |
| | | | | gaagacagtggcccctacagaatgttca |
| 59 | | | Cλ Light Chain Constant | GQPKANPTVTLFPPSSEELQANKATLVCLISDFYPGAVTVAWKADGSPVKAGVETTKP |
| | | | Region (IGLC1*02) Amino | SKQSNNKYAASSYLSLTPEQWKSHRSYSCQVTHEGSTVEKTVAPTECS |
| | | | Acid Sequence | |
| 60 | Human Cλ | IGLC2*01 | Cλ Light Chain Constant | ggccagcctaaggccgctccttctgtgaccctgttccccccatcctccgaggaactgc |
| | constant | | Region (IGLC2*01) | aggctaacaaggccaccctcgtgtgcctgatcagcgacttctaccctggcgccgtgac |
| | region | | Nucleotide Sequence | cgtggcctggaaggctgatagctctcctgtgaaggccggcgtggaaaccaccacccct |
| | | | Version A | tccaagcagtccaacaacaaatacgccgcctcctcctacctgtccctgacccctgagc |
| | | | | agtggaagtcccaccggtcctacagctgccaagtgacccacgagggctccaccgtgga |
| | | | | aaagaccgtggctcctaccgagtgctcc |
| 61 | | | Cλ Light Chain Constant | ggccagcctaaagctgcccccagcgtcaccctgtttcctccctccagcgaggagctcc |
| | | | Region (IGLC2*01) | aggccaacaaggccaccctcgtgtgcctgatctccgacttctatcccggcgctgtgac |
| | | | Nucleotide Sequence | cgtggcttggaaagccgactccagccctgtcaaagccggcgtggagaccaccacaccc |
| | | | Version B | tccaagcagtccaacaacaagtacgccgcctccagctatctctccctgacccctgagc |
| | | | | agtggaagtcccaccggtcctactcctgtcaggtgacccacgagggctccaccgtgga |
| | | | | aaagaccgtcgcccccaccgagtgctcc |
| 62 | | | Cλ Light Chain Constant | GQPKAAPSVTLFPPSSEELQANKATLVCLISDFYPGAVTVAWKADSSPVKAGVETTTP |
| | | | Region (IGLC1*02) Amino | SKQSNNKYAASSYLSLTPEQWKSHRSYSCQVTHEGSTVEKTVAPTECS |
| | | | Acid Sequence | |
| 63 | Human Cλ | IGLC2*02 | Cλ Light Chain Constant | ggtcagcccaaggctgccccctcggtcactctgttcccgccctcctctgaggagcttc |
| | constant | or | Region (IGLC2*02 or | aagccaacaaggccacactggtgtgtctcataagtgacttctacccgggagccgtgac |
| | region | IGLC2*03 | IGLC2*03) Nucleotide | agtggcctggaaggcagatagcagccccgtcaaggcgggagtggagaccaccacaccc |
| | | | Sequence | tccaaacaaagcaacaacaagtacgcggccagcagctatctgagcctgacgcctgagc |
| | | | | agtggaagtcccacagaagctacagctgccaggtcacgcatgaagggagcaccgtgga |
| | | | | gaagacagtggcccctacagaatgttca |
| 64 | | | Cλ Light Chain Constant | GQPKAAPSVTLFPPSSEELQANKATLVCLISDFYPGAVTVAWKADSSPVKAGVETTTP |
| | | | Region (IGLC2*02) Amino | SKQSNNKYAASSYLSLTPEQWKSHRSYSCQVTHEGSTVEKTVAPTECS |
| | | | Acid Sequence | |
| 65 | Human Cλ | IGLC3*01 | Cλ Light Chain Constant | |
| | constant | | Region (IGLC3*01) | acaaggccacactggtgtgtctcataagtgacttctacccgggagccgtgacagttgc |
| | region | | Nucleotide Sequence | ctggaaggcagatagcagccccgtcaaggcgggggtggagaccaccacaccctccaaa |
| | | | | caaagcaacaacaagtacgcggccagcagctacctgagcctgacgcctgagcagtgga |
| | | | | agtcccacaaaagctacagctgccaggtcacgcatgaagggagcaccgtggagaagac |
| | | | | agttgcccctacggaatgttca |
| 66 | | | Cλ Light Chain Constant | PKAAPSVTLFPPSSEELQANKATLVCLISDFYPGAVTVAWKADSSPVKAGVETTTPSK |
| | | | Region (IGLC3*01) Amino | QSNNKYAASSYLSLTPEQWKSHKSYSCQVTHEGSTVEKTVAPTECS |
| | | | Acid Sequence | |
| 67 | Human Cλ | IGLC3*02 | Cλ Light Chain Constant | ggtcagcccaaggctgccccctcggtcactctgttcccaccctcctctgaggagcttc |
| | constant | | Region (IGLC3*02) | aagccaacaaggccacactggtgtgtctcataagtgacttctacccggggccagtgac |
| | region | | Nucleotide Sequence | agttgcctggaaggcagatagcagccccgtcaaggcgggggtggagaccaccacaccc |
| | | | | tccaaacaaagcaacaacaagtacgcggccagcagctacctgagcctgacgcctgagc |
| | | | | agtggaagtcccacaaaagctacagctgccaggtcacgcatgaagggagcaccgtgga |
| | | | | gaagacagtggcccctacggaatgttca |
| 68 | | | Cλ Light Chain Constant | GQPKAAPSVTLFPPSSEELQANKATLVCLISDFYPGPVTVAWKADSSPVKAGVETTTP |
| | | | Region (IGLC1*02) Amino | SKQSNNKYAASSYLSLTPEQWKSHKSYSCQVTHEGSTVEKTVAPTECS |
| | | | Acid Sequence | |
| 69 | Human Cλ | IGLC3*03 | Cλ Light Chain Constant | ggtcagcccaaggctgccccctcggtcactctgttcccaccctcctctgaggagcttc |
| | constant | | Region (IGLC3*03) | aagccaacaaggccacactggtgtgtctcataagtgacttctacccgggagccgtgac |
| | region | | Nucleotide Sequence | agtggcctggaaggcagatagcagccccgtcaaggegggagtggagaccaccacaccc |
| | | | | tccaaacaaagcaacaacaagtacgcggccagcagctacctgagcctgacgcctgagc |
| | | | | agtggaagtcccacaaaagctacagctgccaggtcacgcatgaagggagcaccgtgga |
| | | | | gaagacagtggcccctacagaatgttca |
| 70 | | | Cλ Light Chain Constant | GQPKAAPSVTLFPPSSEELQANKATLVCLISDFYPGAVTVAWKADSSPVKAGVETTTP |
| | | | Region (IGLC3*03) Amino | SKQSNNKYAASSYLSLTPEQWKSHKSYSCQVTHEGSTVEKTVAPTECS |
| | | | Acid Sequence | |
| 71 | Human Cλ | IGLC3*04 | Cλ Light Chain Constant | ggtcagcccaaggctgccccctcggtcactctgttcccgccctcctctgaggagcttc |
| | constant | | Region (IGLC3*04) | aagccaacaaggccacactggtgtgtctcataagtgacttctacccgggagccgtgac |
| | region | | Nucleotide Sequence | agtggcctggaaggcagatagcagccccgtcaaggegggagtggagaccaccacaccc |
| | | | | tccaaacaaagcaacaacaagtacgcggccagcagctacctgagcctgacgcctgagc |
| | | | | agtggaagtcccacagaagctacagctgccaggtcacgcatgaagggagcaccgtgga |
| | | | | gaagacagtggcccctacagaatgttca |
| 72 | | | Cλ Light Chain Constant | GQPKAAPSVTLFPPSSEELQANKATLVCLISDFYPGAVTVAWKADSSPVKAGVETTTP |
| | | | Region (IGLC3*04) Amino | SKQSNNKYAASSYLSLTPEQWKSHRSYSCQVTHEGSTVEKTVAPTECS |
| | | | Acid Sequence | |
| 73 | Human Cλ | IGLC6*01 | Cλ Light Chain Constant | ggtcagcccaaggctgccccatcggtcactctgttcccgccctcctctgaggagcttc |
| | constant | | Region (IGLC6*01) | aagccaacaaggccacactggtgtgcctgatcagtgacttctacccgggagctgtgaa |
| | region | | Nucleotide Sequence | agtggcctggaaggcagatggcagccccgtcaacaegggagtggagaccaccacaccc |
| | | | | tccaaacagagcaacaacaagtacgcggccagcagctacctgagcctgacgcctgagc |
| | | | | agtggaagtcccacagaagctacagctgccaggtcacgcatgaagggagcaccgtgga |
| | | | | gaagacagtggcccctgcagaatgttca |
| 74 | | | Cλ Light Chain Constant | GQPKAAPSVTLFPPSSEELQANKATLVCLISDFYPGAVKVAWKADGSPVNTGVETTTP |
| | | | Region (IGLC6*01) Amino | SKQSNNKYAASSYLSLTPEQWKSHRSYSCQVTHEGSTVEKTVAPAECS |
| | | | Acid Sequence | |
| 75 | Human Cλ | IGLC7*01 | Cλ Light Chain Constant | ggtcagcccaaggctgccccatcggtcactctgttcccaccctcctctgaggagcttc |
| | constant | or | Region (IGLC7*01 or | aagccaacaaggccacactggtgtgtctcgtaagtgacttctacccgggagccgtgac |
| | region | IGLC7*02 | IGLC7*02) Nucleotide | agtggcctggaaggcagatggcagccccgtcaaggtgggagtggagaccaccaaaccc |
| | | | Sequence | tccaaacaaagcaacaacaagtatgcggccagcagctacctgagcctgaegccegage |
| | | | | agtggaagtcccacagaagctacagctgccgggtcacgcatgaagggagcaccgtgga |
| | | | | gaagacagtggcccctgcagaatgctct |
| 76 | | | Cλ Light Chain Constant | GQPKAAPSVTLFPPSSEELQANKATLVCLVSDFYPGAVTVAWKADGSPVKVGVETTKP |
| | | | Region (IGLC7*01) Amino | SKQSNNKYAASSYLSLTPEQWKSHRSYSCRVTHEGSTVEKTVAPAECS |
| | | | Acid Sequence | |
| 77 | Human Cλ | IGLC7*03 | Cλ Light Chain Constant | GGTCAGCCCAAGGCTGCCCCCTCGGTCACTCTGTTCCCACCCTCCTCTGAGGAGCTTC |
| | constant | | Region (IGLC7*03) | AAGCCAACAAGGCCACACTGGTGTGTCTCGTAAGTGACTTCAACCCGGGAGCCGTGAC |
| | region | | Nucleotide Sequence | AGTGGCCTGGAAGGCAGATGGCAGCCCCGTCAAGGTGGGAGTGGAGACCACCAAACCC |
| | | | | TCCAAACAAAGCAACAACAAGTATGCGGCCAGCAGCTACCTGAGCCTGACGCCCGAGC |
| | | | | AGTGGAAGTCCCACAGAAGCTACAGCTGCCGGGTCACGCATGAAGGGAGCACCGTGGA |
| | | | | GAAGACAGTGGCCCCTGCAGAATGCTCT |
| 78 | | | Cλ Light Chain Constant | GQPKAAPSVTLFPPSSEELQANKATLVCLVSDFNPGAVTVAWKADGSPVKVGVETTKP |
| | | | Region (IGLC7*03) Amino | SKQSNNKYAASSYLSLTPEQWKSHRSYSCRVTHEGSTVEKTVAPAECS |
| | | | Acid Sequence | |

TABLE K
KY1044 antibody sequences

| SEQ ID | | SEQUENCE |
|---|---|---|
| 1 | Amino acid sequence | GVTFDDYG |
| | of HCDR1 of KY1044 | |
| 2 | Amino acid sequence | INWNGGDT |
| | of HCDR2 of KY1044 | |
| 3 | Amino acid sequence | ARDFYGSGSYYHVPFDY |
| | of HCDR3 of KY1044 | |
| 4 | Nucleic acid sequence | GAGGTGCAGCTGGTGGAGTCTGGGGGAGGTGTGGTACGGCCTGGGGGGTCCCTGAGACTCTCCTGTGTAGCCTCTGGAGTCA |
| | of VH of KY1044 | CCTTTGATGATTATGGCATGAGCTGGGTCCGCCAAGCTCCAGGGAAGGGGCTGGARTGGGTCTCTGGTATTAATTGGAATGG |
| | | TGGCGACACAGATTATTCAGACTCTGTGAAGGGCCGATTCACCATCTCCAGAGACAACGCCAAGAACTCCCTGTATCTACAA |
| | | ATGAATAGTCTGAGAGCCGAGGACACGGCCTTGTATTACTGTGCGAGGGATTTCTATGGTTCGGGGAGTTATTATCACGTTC |
| | | CTTTTGACTACTGGGGCCAGGGAATCCTGGTCACCGTCTCCTCA |
| 5 | Amino acid sequence | EVQLVESGGGVVRPGGSLRLSCVASGVTFDDYGMSWVRQAPGKGLEWVSGINWNGGDTDYSDSVKGRFTISRDNAKNSLYLQ |
| | of VH of KY1044 | MNSLRAEDTALYYCARDFYGSGSYYHVPFDYWGQGILVTVSS |
| 6 | Nucleic acid sequence | GAGGTGCAGCTGGTGGAGTCTGGGGGAGGTGTGGTACGGCCTGGGGGGTCCCTGAGACTCTCCTGTGTAGCCTCTGGAGTCA |
| | of KY1044 heavy chain | CCTTTGATGATTATGGCATGAGCTGGGTCCGCCAAGCTCCAGGGAAGGGGCTGGARTGGGTCTCTGGTATTAATTGGAATGG |
| | | TGGCGACACAGATTATTCAGACTCTGTGAAGGGCCGATTCACCATCTCCAGAGACAACGCCAAGAACTCCCTGTATCTACAA |
| | | ATGAATAGTCTGAGAGCCGAGGACACGGCCTTGTATTACTGTGCGAGGGATTTCTATGGTTCGGGGAGTTATTATCACGTTC |
| | | CTTTTGACTACTGGGGCCAGGGAATCCTGGTCACCGTCTCCTCAGCCAGCACCAAGGGCCCCTCTGTGTTCCCTCTGGCCCC |
| | | TTCCAGCAAGTCCACCTCTGGCGGAACAGCCGCTCTGGGCTGCCTCGTGAAGGACTACTTCCCCGAGCCTGTGACCGTGTCC |
| | | TGGAACTCTGGCGCTCTGACCAGCGGAGTGCACACCTTCCCTGCTGTGCTGCAGTCCTCCGGCCTGTACTCCCTGTCCTCCG |
| | | TCGTGACCGTGCCTTCCAGCTCTCTGGGCACCCAGACCTACATCTGCAACGTGAACCACAAGCCCTCCAACACCAAGGTGGA |
| | | CAAGAAGGTGGAACCCAAGTCCTGCGACAAGACCCACACCTGTCCCCCTTGTCCTGCCCCTGAACTGCTGGGCGGACCTTCC |
| | | GTGTTCCTGTTCCCCCCAAAGCCCAAGGACACCCTGATGATCTCCCGGACCCCCGAAGTGACCTGCGTGGTGGTGGATGTGT |
| | | CCCACGAGGACCCTGAAGTGAAGTTCAATTGGTACGTGGACGGCGTGGAAGTGCACAACGCCAAGACCAAGCCTAGAGAGGA |
| | | ACAGTACAACTCCACCTACCGGGTGGTGTCCGTGCTGACCGTGCTGCACCAGGATTGGCTGAACGGCAAAGAGTACAAGTGC |
| | | AAGGTGTCCAACAAGGCCCTGCCTGCCCCCATCGAAAAGACCATCTCCAAGGCCAAGGGCCAGCCCCGGGAACCCCAGGTGT |
| | | ACACACTGCCCCCTAGCAGGGACGAGCTGACCAAGAACCAGGTGTCCCTGACCTGTCTCGTGAAAGGCTTCTACCCCTCCGA |
| | | TATCGCCGTGGAATGGGAGTCCAACGGCCAGCCTGAGAACAACTACAAGACCACCCCCCCTGTGCTGGACTCCGACGGCTCA |
| | | TTCTTCCTGTACAGCAAGCTGACAGTGGACAAGTCCCGGTGGCAGCAGGGCAACGTGTTCTCCTGCTCCGTGATGCACGAGG |
| | | CCCTGCACAACCACTACACCCAGAAGTCCCTGTCCCTGAGCCCCGGCAAGTGATGA |
| 7 | Amino acid sequence | EVQLVESGGGVVRPGGSLRLSCVASGVTFDDYGMSWVRQAPGKGLEWVSGINWNGGDTDYSDSVKGRFTISRDNAKNSLYLQ |
| | of KY1044 heavy chain | MNSLRAEDTALYYCARDFYGSGSYYHVPFDYWGQGILVTVSSASTKGPSVFPLAPSSKSTSGGTAALGCLVKDYFPEPVTVS |
| | | WNSGALTSGVHTFPAVLQSSGLYSLSSWTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 8 | Amino acid sequence | QSVSRSY |
| | of LCDR1 of KY1044 | |
| 9 | Amino acid sequence | GAS |
| | of LCDR2 of KY1044 | |
| 10 | Amino acid sequence | HQYDMSPFT |
| | of LCDR3 of KY1044 | |
| 11 | Nucleic acid sequence | GAAATTGTGTTGACGCAGTCTCCAGGGACCCTGTCTTTGTCTCCAGGGGAAAGAGCCACCCTCTCCTGCAGGGCCAGTCAGA |
| | of VL of KY1044 | GTGTTAGCAGAAGCTACTTAGCCTGGTACCAGCAGAAACGTGGCCAGGCTCCCAGGCTCCTCATCTATGGTGCATCCAGCAG |
| | | GGCCACTGGCATCCCAGACAGGTTCAGTGGCGATGGGTCTGGGACAGACTTCACTCTCTCCATCAGCAGACTGGAGCCTGAA |
| | | GATTTTGCAGTGTATTACTGTCACCAGTATGATATGTCACCATTCACTTTCGGCCCTGGGACCAAAGTGGATATCAAA |
| 12 | Amino acid sequence | EIVLTQSPGTLSLSPGERATLSCRASQSVSRSYLAWYQQKRGQAPRLLIYGASSRATGIPDRFSGDGSGTDFTLSISRLEPE |
| | of VL of KY1044 | DFAVYYCHQYDMSPFTFGPGTKVDIK |
| 13 | Nucleic acid sequence | GAAATTGTGTTGACGCAGTCTCCAGGGACCCTGTCTTTGTCTCCAGGGGAAAGAGCCACCCTCTCCTGCAGGGCCAGTCAGA |
| | of KY1044 light chain | GTGTTAGCAGAAGCTACTTAGCCTGGTACCAGCAGAAACGTGGCCAGGCTCCCAGGCTCCTCATCTATGGTGCATCCAGCAG |
| | | GGCCACTGGCATCCCAGACAGGTTCAGTGGCGATGGGTCTGGGACAGACTTCACTCTCTCCATCAGCAGACTGGAGCCTGAA |
| | | GATTTTGCAGTGTATTACTGTCACCAGTATGATATGTCACCATTCACTTTCGGCCCTGGGACCAAAGTGGATATCAAAcgta |
| | | cggtggccgctccctccgtgttcatcttcccaccttccgacgagcagctgaagtccggcaccgcttctgtcgtgtgcctgct |
| | | gaacaacttctacccccgcgaggccaaggtgcagtggaaggtggacaacgccctgcagtccggcaactcccaggaatccgtg |
| | | accgagcaggactccaaggacagcacctactccctgtcctccaccctgaccctgtccaaggccgactacgagaagcacaagg |
| | | tgtacgcctgcgaagtgacccaccagggcctgtctagccccgtgaccaagtctttcaaccggggcgagtgt |
| 14 | Amino acid sequence | EIVLTQSPGTLSLSPGERATLSCRASQSVSRSYLAWYQQKRGQAPRLLIYGASSRATGIPDRFSGDGSGTDFTLSISRLEPE |
| | of KY1044 light chain | DFAVYYCHQYDMSPFTFGPGTKVDIKRTVAAPSVFIFPPSDEQLKSGTASVVCLLNNFYPREAKVQWKVDNALQSGNSQESV |
| | | TEQDSKDSTYSLSSTLTLSKADYEKHKVYACEVTHQGLSSPVTKSFNRGEC |

What is claimed is:

1. A method for prognosis of a cancerous solid tumour in a patient, comprising
providing a sample of tumour core tissue obtained from the patient,
determining one or more of the following biomarkers in said sample:
(i) ratio of the number of ICOS FOXP3 double positive cells within a defined radius of influence around ICOS single positive cells to the total number of ICOS single positive cells,
(ii) mean distance between each ICOS positive FOXP3 negative cell and its nearest ICOS FOXP3 double positive cell,
(iii) proportion of FOXP3 positive cells which are ICOS positive, and
(iv) density of ICOS positive cells, and
providing a prognosis for the patient based on said one or more biomarkers, wherein a shorter duration of patient survival, recurrence free survival (RFS), progression free survival (PFS) or time to progression (TTP) is indicated by
a greater ratio of the number of ICOS FOXP3 double positive cells within a defined radius of influence around ICOS single positive cells to the total number of ICOS single positive cells,
a shorter mean distance between each ICOS positive FOXP3 negative cell and its nearest ICOS FOXP3 double positive cell,
a higher proportion of FOXP3 positive cells which are ICOS positive, and/or
a higher density of ICOS positive cells.

2. A method according to claim 1, comprising determining the ratio of the number of ICOS FOXP3 double positive cells within a defined radius of influence around ICOS single positive cells to the total number of ICOS single positive cells, and
comparing said ratio against a reference value, wherein
a ratio higher than the reference value indicates a prognosis of shorter duration of survival, RFS, PFS or TTP.

3. A method according to claim 2, wherein the tumour is hepatocellular carcinoma and the reference value is a ratio of 0.1 ratio of the number of ICOS FOXP3 double positive cells within a defined radius of influence around ICOS single positive cells to the total number of ICOS single positive cells.

4. A method according to any of claims 1 to 3, comprising determining the mean distance between each ICOS positive FOXP3 negative cell and its nearest ICOS FOXP3 double positive cell, and
comparing said distance against a reference value, wherein
a distance less than the reference value indicates a prognosis of shorter duration of survival, RFS, PFS or TTP.

5. A method according to claim 4, wherein the tumour is hepatocellular carcinoma and the reference value is a mean distance of 105 μm between each ICOS positive FOXP3 negative cell and its nearest ICOS FOXP3 double positive cell.

6. A method according to any of claims 1 to 5, comprising determining the proportion of FOXP3 positive cells which are ICOS positive, and
comparing said proportion against a reference value, wherein
a proportion higher than the reference value indicates a prognosis of shorter duration of survival, RFS, PFS or TTP.

7. A method according to claim 6, wherein the tumour is hepatocellular carcinoma and the reference value is a half of FOXP3 positive cells being ICOS positive.

8. A method according to any of claims 1 to 7, comprising determining the density of ICOS positive cells, and
comparing said density against a reference value, wherein
a density higher than the reference value indicates a prognosis of shorter duration of survival, RFS, PFS or TTP.

9. A method according to claim 7, wherein the tumour is hepatocellular carcinoma and the reference value is a density of 120 ICOS positive cells per mm².

10. A method according to claim 7, wherein the tumour is hepatocellular carcinoma associated with hepatitis B virus infection or is stage 2 or later hepatocellular carcinoma, and wherein the reference value is a density of 100 ICOS positive cells per mm².

11. A method according to any of claims 1 to 10, comprising determining from said one or more biomarkers that the patient has a prognosis of shorter duration of survival, RFS, PFS or TTP.

12. A method according to claim 11, comprising determining the ratio of the number of ICOS FOXP3 double positive cells within a defined radius of influence around ICOS single positive cells to the total number of ICOS single positive cells, and
comparing said ratio against a reference value, wherein a ratio higher than the reference value indicates a prognosis of shorter duration of survival, RFS, PFS or TTP,
determining that said ratio is higher than the reference value, and
providing a prognosis of shorter duration of survival, RFS, PFS or TTP.

13. A method according to any of claims 1 to 10, comprising determining from said one or more biomarkers that the patient has a prognosis of longer duration of survival, RFS, PFS or TTP.

14. A method according to claim 13, comprising determining the ratio of the number of ICOS FOXP3 double positive cells within a defined radius of influence around ICOS single positive cells to the total number of ICOS single positive cells, and
comparing said ratio against a reference value, wherein a ratio higher than the reference value indicates a prognosis of shorter duration of survival, RFS, PFS or TTP,
determining that said ratio is lower than the reference value, and
providing a prognosis of longer duration of survival, RFS, PFS or TTP.

15. Use of a biomarker for prognosis of a cancerous solid tumour in a patient, wherein the biomarker is one or more of the following as determined in tumour core tissue from the patient:
(i) ratio of the number of ICOS FOXP3 double positive cells within a defined radius of influence around ICOS single positive cells to the total number of ICOS single positive cells,
(ii) mean distance between each ICOS positive FOXP3 negative cell and its nearest ICOS FOXP3 double positive cell,
(iii) proportion of FOXP3 positive cells which are ICOS positive, and
(iv) density of ICOS positive cells.

16. A method of determining likelihood of a cancerous solid tumour in a patient to respond to an anti-ICOS and/or anti-TReg immunotherapeutic agent, comprising
providing a sample of tumour core tissue obtained from the patient, and
determining one or more of the following biomarkers in said sample:
(i) ratio of the number of ICOS FOXP3 double positive cells within a defined radius of influence around ICOS single positive cells to the total number of ICOS single positive cells,
(ii) mean distance between each ICOS positive FOXP3 negative cell and its nearest ICOS FOXP3 double positive cell,
(iii) proportion of FOXP3 positive cells which are ICOS positive, and
(iv) density of ICOS positive cells, wherein
a greater likelihood of the patient to respond to the immunotherapeutic agent is indicated by
a greater ratio of the number of ICOS FOXP3 double positive cells within a defined radius of influence around ICOS single positive cells to the total number of ICOS single positive cells,
a shorter mean distance between each ICOS positive FOXP3 negative cell and its nearest ICOS FOXP3 double positive cell,
a higher proportion of FOXP3 positive cells which are ICOS positive, and/or
a higher density of ICOS positive cells.

17. A method according to claim 16, comprising determining the ratio of the number of ICOS FOXP3 double positive cells within a defined radius of influence around ICOS single positive cells to the total number of ICOS single positive cells, and
comparing said number against a reference value, wherein
a ratio higher than the reference value indicates an increased likelihood of responding to the immunotherapeutic agent.

18. A method according to claim 17, wherein the tumour is hepatocellular carcinoma and the reference value is a ratio of 0.1 ICOS FOXP3 double positive cells within a defined radius of influence around ICOS single positive cells to the total number of ICOS single positive cells.

19. A method according to any of claims 16 to 18, comprising determining the mean distance between each ICOS positive FOXP3 negative cell and its nearest ICOS FOXP3 double positive cell, and
comparing said distance against a reference value, wherein
a distance less than the reference value indicates an increased likelihood of responding to the immunotherapeutic agent.

20. A method according to claim 19, wherein the tumour is hepatocellular carcinoma and the reference value is a mean distance of 105 μm between each ICOS positive FOXP3 negative cell and its nearest ICOS FOXP3 double positive cell.

21. A method according to any of claims 16 to 20, comprising determining the proportion of FOXP3 positive cells which are ICOS positive, and
comparing said proportion against a reference value, wherein
a proportion higher than the reference value indicates an increased likelihood of responding to the immunotherapeutic agent.

22. A method according to claim 21, wherein the tumour is hepatocellular carcinoma and the reference value is a half of FOXP3 positive cells being ICOS positive.

23. A method according to any of claims 16 to 22, comprising determining the density of ICOS positive cells, and
comparing said density against a reference value, wherein
a density higher than the reference value indicates an increased likelihood of responding to the immunotherapeutic agent.

24. A method according to claim 23, wherein the tumour is hepatocellular carcinoma and the reference value is a density of 120 ICOS positive cells per mm².

25. A method according to claim 23, wherein the tumour is hepatocellular carcinoma associated with hepatitis B virus infection or is stage 2 or later hepatocellular carcinoma, and wherein the reference value is a density of 100 ICOS positive cells per mm².

26. A method according to any of claims 16 to 25, comprising identifying the patient as having an increased likelihood of responding to the immunotherapeutic agent and optionally thereby selecting an anti-ICOS and/or anti-TReg immunotherapeutic agent for treatment of said patient.

27. A method according to claim 26, comprising determining the ratio of the number of ICOS FOXP3 double positive cells within a defined radius of influence around ICOS single positive cells to the total number of ICOS single positive cells,
comparing said ratio against a reference value, wherein a ratio higher than the reference value indicates an increased likelihood of responding to the immunotherapeutic agent,
determining that said ratio is higher than the reference value, and thereby identifying the patient as having an increased likelihood of responding to the immunotherapeutic agent.

28. A method according to claim 26 or claim 27, comprising administering the anti-ICOS and/or anti-TReg immunotherapeutic agent to said patient.

29. Use of a biomarker for determining likelihood of a cancerous tumour in a patient to respond to an anti-ICOS and/or anti-TReg immunotherapeutic agent, wherein the biomarker is one or more of the following as determined in tumour core tissue from the patient:
(i) ratio of the number of ICOS FOXP3 double positive cells within a defined radius of influence around ICOS single positive cells to the total number of ICOS single positive cells,
(ii) mean distance between each ICOS positive FOXP3 negative cell and its nearest ICOS FOXP3 double positive cell,
(iii) proportion of FOXP3 positive cells which are ICOS positive, and
(iv) density of ICOS positive cells.

30. A method of treating a cancerous solid tumour in a patient, wherein the tumour has been determined to comprise one or more of the following biomarkers:
ratio of the number of ICOS FOXP3 double positive cells within a defined radius of influence around ICOS single positive cells to the total number of ICOS single positive cells, wherein said ratio is higher than a reference value,
a mean distance between each ICOS positive FOXP3 negative cell and its nearest ICOS FOXP3 double positive cell, wherein said distance is less than a reference value,
a proportion of FOXP3 positive cells which are ICOS positive, wherein said proportion is higher than a reference value, and
a density of ICOS positive cells, wherein said density is higher than a reference value,
the method comprising administering an anti-ICOS and/or anti-TReg immunotherapeutic agent to the patient.

31. An anti-ICOS and/or anti-TReg immunotherapeutic agent for use in a method of treating a cancerous solid tumour in a patient, wherein the tumour has been determined to comprise one or more of the following biomarkers:
a ratio of the number of ICOS FOXP3 double positive cells within a defined radius of influence around ICOS single positive cells to the total number of ICOS single positive cells, wherein said ratio is higher than a reference value,
a mean distance between each ICOS positive FOXP3 negative cell and its nearest ICOS FOXP3 double positive cell, wherein said distance is less than a reference value,
a proportion of FOXP3 positive cells which are ICOS positive, wherein said proportion is higher than a reference value, and
a density of ICOS positive cells, wherein said density is higher than a reference value.

32. A method according to claim 30, or an agent for use according to claim 31, wherein the tumour is hepatocellular carcinoma and the tumour has been determined to comprise the following biomarkers:
a ratio of the number of ICOS FOXP3 double positive cells within a defined radius of influence around ICOS single positive cells to the total number of ICOS single positive cells, wherein said ratio is higher than 0.1,
a mean distance between each ICOS positive FOXP3 negative cell and its nearest ICOS FOXP3 double positive cell, wherein said distance is less than 105 μm,
a proportion of FOXP3 positive cells which are ICOS positive, wherein said proportion is higher than half, and
a density of ICOS positive cells, wherein said density is higher than 120 cells per mm².

33. A method according to claim 30, or an agent for use according to claim 31, wherein the tumour is hepatocellular carcinoma associated with hepatitis B virus infection or is grade 2 or later hepatocellular carcinoma, and wherein the tumour has been determined to comprise the following biomarkers:
a ratio of the number of ICOS FOXP3 double positive cells within a defined radius of influence around ICOS single positive cells to the total number of ICOS single positive cells, wherein said ratio is higher than 0.1,
a mean distance between each ICOS positive FOXP3 negative cell and its nearest ICOS FOXP3 double positive cell, wherein said distance is less than 105 μm,
a proportion of FOXP3 positive cells which are ICOS positive, wherein said proportion is higher than half, and
a density of ICOS positive cells, wherein said density is higher than 100 cells per mm².

34. An anti-ICOS and/or anti-TReg immunotherapeutic agent for use in a method of treating a cancerous solid tumour in a patient, the method comprising
selecting an anti-ICOS and/or anti-TReg immunotherapeutic agent for treatment of a patient as defined in claim 26, and
administering the anti-ICOS and/or anti-TReg immunotherapeutic agent to the patient.

35. A method of monitoring a patient's response to an anti-ICOS and/or anti-TReg immunotherapeutic agent for a cancerous solid tumour, comprising
providing a sample of tumour core tissue obtained from a patient who has received an anti-ICOS and/or anti-TReg immunotherapeutic agent,
determining one or more of the following biomarkers in said sample:
(i) ratio of the number of ICOS FOXP3 double positive cells within a defined radius of influence around ICOS single positive cells to the total number of ICOS single positive cells,
(ii) mean distance between each ICOS positive FOXP3 negative cell and its nearest ICOS FOXP3 double positive cell,
(iii) proportion of FOXP3 positive cells which are ICOS positive, and
(iv) density of ICOS positive cells,
comparing said one or more biomarkers in said sample against the same one or more biomarkers in a sample of tumour core tissue obtained from the patient prior to administration of said immunotherapeutic agent, and
determining whether a change has occurred in said one or more biomarkers and thereby assessing whether the patient is responding to the immunotherapeutic agent, wherein response is indicated by one or more of the following changes from the sample prior to administration of said immunotherapeutic agent:
a reduction in the ratio of the number of ICOS FOXP3 double positive cells within a defined radius of influence around ICOS single positive cells to the total number of ICOS single positive cells,
an increase in the mean distance between each ICOS positive FOXP3 negative cell and its nearest ICOS FOXP3 double positive cell,
a reduction in the proportion of FOXP3 positive cells which are ICOS positive, and/or
a reduction in the density of ICOS positive cells.

36. A method according to claim 35, comprising
measuring a reduction in the ratio of the number of ICOS FOXP3 double positive cells within a defined radius of influence around ICOS single positive cells to the total number of ICOS single positive cells compared with prior to therapy, and
prescribing continued treatment with the anti-ICOS and/or anti-TReg immunotherapeutic agent for the patient.

37. A method according to claim 35 or claim 36, comprising
measuring an increase in the mean distance between each ICOS positive FOXP3 negative cell and its nearest ICOS FOXP3 double positive cell, and
prescribing continued treatment with the anti-ICOS and/or anti-TReg immunotherapeutic agent for the patient.

38. A method according to any of claims 35 to 37, comprising
measuring a reduction in the proportion of FOXP3 positive cells which are ICOS positive, and
prescribing continued treatment with the anti-ICOS and/or anti-TReg immunotherapeutic agent for the patient.

39. A method according to any of claims 35 to 38, comprising
measuring a reduction in the density of ICOS positive cells, and
prescribing continued treatment with the anti-ICOS and/or anti-TReg immunotherapeutic agent for the patient.

40. Use of a biomarker for monitoring a patient's response to an anti-ICOS and/or anti-TReg immunotherapeutic agent for a cancerous solid tumour, wherein the biomarker is one or more of the following as determined in tumour core tissue from the patient:
(i) ratio of the number of ICOS FOXP3 double positive cells within a defined radius of influence around ICOS single positive cells to the total number of ICOS single positive cells,
(ii) mean distance between each ICOS positive FOXP3 negative cell and its nearest ICOS FOXP3 double positive cell,
(iii) proportion of FOXP3 positive cells which are ICOS positive, and
(iv) density of ICOS positive cells,
wherein response to the immunotherapeutic agent is indicated by one or more of the following changes as compared with a sample of tumour core tissue obtained prior to administration of the immunotherapeutic agent:
a reduction in the ratio of the number of ICOS FOXP3 double positive cells within a defined radius of influence around ICOS single positive cells to the total number of ICOS single positive cells,
an increase in the mean distance between each ICOS positive FOXP3 negative cell and its nearest ICOS FOXP3 double positive cell,
a reduction in the proportion of FOXP3 positive cells which are ICOS positive, and
a reduction in the density of ICOS positive cells.

41. A method of identifying a reference value for classifying patients with cancerous solid tumours according to their predicted prognosis or predicted response to an anti-ICOS and/or anti-TReg immunotherapeutic agent, comprising
providing samples of tumour core tissue obtained from each of a population of patients with cancerous solid tumours of the same type for whom disease outcome is known or response to the anti-ICOS and/or anti-TReg immunotherapeutic agent is known,
determining one or more of the following biomarkers in each of said samples:
(i) ratio of the number of ICOS FOXP3 double positive cells within a defined radius of influence around ICOS single positive cells to the total number of ICOS single positive cells,
(ii) mean distance between each ICOS positive FOXP3 negative cell and its nearest ICOS FOXP3 double positive cell,
(iii) proportion of FOXP3 positive cells which are ICOS positive, and
(iv) density of ICOS positive cells,
pairing data for each of said one or more biomarkers with data for disease outcome or response to the anti-ICOS and/or anti-TReg immunotherapeutic agent in each patient, and
grouping data for each of said one or more biomarkers to identify a numerical cut-off which defines two groups of statistically significant data for disease outcome or response to the anti-ICOS and/or anti-TReg immunotherapeutic agent,
wherein said cut-off represents a reference value for classifying patients with the same type of cancerous solid tumours according to their predicted prognosis or predicted response to the anti-ICOS and/or anti-TReg immunotherapeutic agent.

42. A method according to claim 41, wherein the samples are obtained from a population of patients with liver cancer, renal cell cancer, head and neck cancer, melanoma, non-small cell lung cancer, bladder cancer, ovarian cancer, cervical cancer, gastric cancer, pancreatic cancer, breast cancer (including triple negative breast cancer), carcinoma, leiomyosarcoma, anal cancer, squamous cell cancer and oesophageal cancer.

43. A method according to claim 42, wherein the samples are obtained from a population of patients with hepatocellular carcinoma.

44. A method, use or an immunotherapeutic agent for use according to any of claims 16 to 43, wherein the immunotherapeutic agent is an anti-ICOS antibody.

45. A method, use or an immunotherapeutic agent for use according to any of claims 16 to 44, wherein the immunotherapeutic agent selectively depletes or inhibits TRegs.

46. A method, use or an immunotherapeutic agent for use according to any of claims 16 to 45, wherein the immunotherapeutic agent is an antibody which binds TRegs and mediates cellular effector functions.

47. A method, use or immunotherapeutic agent for use according to claim 46, wherein the antibody is an IgG comprising an Fc region which mediates cellular effector functions.

48. A method, use or immunotherapeutic agent for use according to any of claims 45 to 47, wherein the immunotherapeutic agent is an antibody which binds ICOS, CD25, CCR8, CTLA-4, GITR or an MHC displayed epitope of FOXP3.

49. A method, use or immunotherapeutic agent for use according to claim 48, wherein the antibody binds ICOS.

50. A method, use or immunotherapeutic agent for use according to claim 44 or claim 49, wherein the one or more biomarkers comprise the density of ICOS positive cells.

51. A method, use or immunotherapeutic agent for use according to claim 49 or claim 50, wherein the antibody comprises an ICOS binding site comprising the CDRs of KY1044, wherein
HCDR1 is SEQ ID NO: 1
HCDR2 is SEQ ID NO: 2
HCDR3 is SEQ ID NO: 3
LCDR1 is SEQ ID NO: 8
LCDR2 is SEQ ID NO: 9 and
LCDR3 is SEQ ID NO: 10.

52. A method, use or immunotherapeutic agent for use according to any of claims 49 to 51, wherein the antibody comprises a VH domain amino acid sequence at least 90% identical to the KY1044 VH domain SEQ ID NO: 5 and a VL domain amino acid sequence at least 90% identical to the KY1044 VL domain SEQ ID NO: 12.

53. A method, use or immunotherapeutic agent for use according to claim 52, wherein antibody comprises the KY1044 VH domain SEQ ID NO: 5 and the KY1044 VL domain SEQ ID NO: 12.

54. A method, use or immunotherapeutic agent for use according to claim 53, wherein the antibody is a human IgG1K comprising the KY1044 heavy chain SEQ ID NO: 7 and the KY1044 light chain SEQ ID NO: 14.

55. A method, use or immunotherapeutic agent according to claim 48, wherein the antibody binds CD25, CCR8, CTLA-4, GITR or an MHC displayed epitope of FOXP3.

56. A method, use or immunotherapeutic agent according to claim 55, wherein the one or more biomarkers comprise the ratio of the number of ICOS FOXP3 double positive cells within a defined radius of influence around ICOS single positive cells to the total number of ICOS single positive cells, the mean distance between each ICOS positive FOXP3 negative cell and its nearest ICOS FOXP3 double positive cell, and/or the proportion of FOXP3 positive cells which are ICOS positive.

57. Use of the spatial arrangement of immune cells expressing ICOS and/or FOXP3 in the TME as a biomarker for disease prognosis or for response to treatment with an anti-ICOS and/or anti-TReg immunotherapeutic agent.


SEQUENCE LISTING (78 sequences)

SEQ ID NO: 1 - protein, 8 aa, Homo Sapiens
GVTFDDYG

SEQ ID NO: 2 - protein, 8 aa, Homo Sapiens
INWNGGDT

SEQ ID NO: 3 - protein, 17 aa, Homo Sapiens
ARDFYGSGSYYHVPFDY

SEQ ID NO: 4 - DNA, 372 bp, Homo Sapiens
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctca

SEQ ID NO: 5 - protein, 124 aa, Homo Sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 6 - DNA, 1368 bp, Homo Sapiens
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 7 - protein, 454 aa, Homo Sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 7 aa, Homo Sapiens
QSVSRSY

SEQ ID NO: 9 - protein, 3 aa, Homo Sapiens
GAS

SEQ ID NO: 10 - protein, 9 aa, Homo Sapiens
HQYDMSPFT

SEQ ID NO: 11 - DNA, 324 bp, Homo Sapiens
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaa

SEQ ID NO: 12 - protein, 108 aa, Homo Sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 13 - DNA, 645 bp, Homo Sapiens
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 14 - protein, 215 aa, Homo Sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 990 bp, Homo Sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 330 aa, Homo Sapiens [sequence omitted]

SEQ ID NO: 17 - DNA, 990 bp, Homo Sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 330 aa, Homo Sapiens [sequence omitted]

SEQ ID NO: 19 - DNA, 990 bp, Homo Sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 20 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 990 bp, Homo Sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 22 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 23 - DNA, 990 bp, Homo Sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 978 bp, Homo Sapiens
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - protein, 326 aa, Homo Sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - DNA, 978 bp, Homo Sapiens
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 28 - protein, 326 aa, Homo Sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - DNA, 978 bp, Homo Sapiens
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 326 aa, Homo Sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - DNA, 978 bp, Homo Sapiens
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 32 - protein, 326 aa, Homo Sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 33 - DNA, 981 bp, Homo Sapiens
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 34 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - DNA, 981 bp, Homo Sapiens
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 36 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - DNA, 981 bp, Homo Sapiens
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 38 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 39 - DNA, 981 bp, Homo Sapiens
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 40 - DNA, 981 bp, Homo Sapiens
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 41 - DNA, 981 bp, Homo Sapiens
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 42 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - DNA, 981 bp, Homo Sapiens
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 44 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - DNA, 321 bp, Homo Sapiens
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 46 - protein, 107 aa, Homo Sapiens [sequence omitted]

SEQ ID NO: 47 - DNA, 321 bp, Homo Sapiens
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 48 - protein, 107 aa, Homo Sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 49 - DNA, 321 bp, Homo Sapiens
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 50 - protein, 107 aa, Homo Sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 51 - DNA, 321 bp, Homo Sapiens
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 52 - protein, 107 aa, Homo Sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 53 - DNA, 321 bp, Homo Sapiens
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 54 - protein, 107 aa, Homo Sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 55 - DNA, 312 bp, Homo Sapiens
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 56 - protein, 104 aa, Homo Sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 57 - DNA, 318 bp, Homo Sapiens
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 58 - DNA, 318 bp, Homo Sapiens; identical to SEQ ID NO: 57

SEQ ID NO: 59 - protein, 106 aa, Homo Sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 60 - DNA, 318 bp, Homo Sapiens
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 61 - DNA, 318 bp, Homo Sapiens
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 62 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 63 - DNA, 318 bp, Homo Sapiens
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 64 - protein, 106 aa, Homo Sapiens; identical to SEQ ID NO: 62

SEQ ID NO: 65 - DNA, 312 bp, Homo Sapiens
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 66 - protein, 104 aa, Homo Sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 67 - DNA, 318 bp, Homo Sapiens
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 68 - protein, 106 aa, Homo Sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 69 - DNA, 318 bp, Homo Sapiens
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 70 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 71 - DNA, 318 bp, Homo Sapiens
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 72 - protein, 106 aa, Homo Sapiens; identical to SEQ ID NO: 62

SEQ ID NO: 73 - DNA, 318 bp, Homo Sapiens
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 74 - protein, 106 aa, Homo Sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 75 - DNA, 318 bp, Homo Sapiens
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 76 - protein, 106 aa, Homo Sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 77 - DNA, 318 bp, Homo Sapiens
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 78 - protein, 106 aa, Homo Sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS